SEQUENCE LISTING (1403 sequences)

SEQ ID NO: 1 - protein, 391 aa, Homo sapiens
MAAPDLSTNLQEEATCAICLDYFTDPVMTDCGHNFCRECIRRCWGQPEGPYACPECRELSPQRNLRPNRPLAKMAEMARRLHPPSPVPQGVCPAHREPLAAFCGDELRLLCAACERSGEHWAHRVRPLQDAAEDLKAKLEKSLEHLRKQMQDALLFQAQADETCVLWQDIKDALRRVQDVKLQPPEVVPMELRTVCRVPGLVETLRRFRGDVTLDPDTANPELILSEDRRSVQRGDLRQALPDSPERFDPGPCVLGQERFTSGRHYWEVEVGDRTSWALGVCRENVNRKEKGELSAGNGFWILVFLGSYYNSSERALAPLRDPPRRVGIFLDYEAGHLSFYSATDGSLLFIFPEIPFSGTLRPLFSPLSSSPTPMTICRPKGGSGDTLAPQ

SEQ ID NO: 2 - protein, 436 aa, Homo sapiens
MPGMMEKGPELLGKNRSANGSAKSPAGGGGSGASSTNGGLHYSEPESGCSSDDEHDVGMRVGAEYQARIPEFDPGATKYTDKDNGGMLVWSPYHSIPDARLDEYIAIAKEKHGYNVEQALGMLFWHKHNIEKSLADLPNFTPFPDEWTVEDKVLFEQAFSFHGKSFHRIQQMLPDKTIASLVKYYYSWKKTRSRTSLMDRQARKLANRHNQGDSDDDVEETHPMDGNDSDYDPKKEAKKEGNTEQPVQTSKIGLGRREYQSLQHRHHSQRSKCRPPKGMYLTQEDVVAVSCSPNAANTILRQLDMELISLKRQVQNAKQVNSALKQKMEGGIEEFKPPESNQKINARWTTEEQLLAVQGTDPTGSSDTGSITSCPIIHSNTNSPYCHSEPASTTSSSNTACCPGSSPAASSTPAAGSVHPAPANFKSASTTSYSPC

SEQ ID NO: 3 - protein, 339 aa, Homo sapiens
MAASALYACTKCTQRYPFEELSQGQQLCKECRIAHPIVKCTYCRSEFQQESKTNTICKKCAQNVKQFGTPKPCQYCNIIAAFIGTKCQRCTNSEKKYGPPQTCEQCKQQCAFDRKEEGRRKVDGKLLCWLCTLSYKRVLQKTKEQRKSLGSSHSNSSSSSLTEKDQHHPKHHHHHHHHHHRHSSSHHKISNLSPEEEQGLWKQSHKSSATIQNETPKKKPKLESKPSNGDSSSINQSADSGGTDNFVLISQLKEEVMSLKRLLQQRDQTILEKDKKLTELKADFQYQESNLRTKMNSMEKAHKETVEQLQAKNRELLKQVAALSKGKKFDKSGSILTSP

SEQ ID NO: 4 - protein, 359 aa, Homo sapiens
MDEQDLNEPLAKVSLLKDDLQGAQSEIEAKQEIQHLRKELIEAQELARTSKQKCFELQALLEEERKAYRNQVEESTKQIQVLQAQLQRLHIDTENLREEKDSEITSTRDELLSARDEILLLHQAAAKVASERDTDIASLQEELKKVRAELERWRKAASEYEKEITSLQNSFQLRCQQCEDQQREEATRLQGELEKLRKEWNALETECHSLKRENVLLSSELQRQEKELHNSQKQSLELTSDLSILQMSRKELENQVGSLKEQHLRDSADLKTLLSKAENQAKDVQKEYEKTQTVLSELKLKFEMTEQEKQSITDELKQCKNNLKLLREKGNNPSILQPVPAVFIGLFLAFLFWCFGPLW

SEQ ID NO: 5 - protein, 643 aa, Homo sapiens
MAAVLALRVVAGLAAAALVAMLLEHYGLAGQPSPLPRPAPPRRPHPAPGPGDSNIFWGLQISDIHLSRFRDPGRAVDLEKFCSETIDIIQPALVLATGDLTDAKTKEQLGSRQHEVEWQTYQGILKKTRVMEKTKWLDIKGNHDAFNIPSLDSIKNYYRKYSAVRRDGSFHYVHSTPFGNYSFICVDATVNPGPKRPYNFFGILDKKKMEELLLLAKESSRSNHTIWFGHFTTSTILSPSPGIRSIMSSAIAYLCGHLHTLGGLMPVLHTRHFQGTLELEVGDWKDNRRYRIFAFDHDLFSFADLIFGKWPVVLITNPKSLLYSCGEHEPLERLLHSTHIRVLAFSLSSITSVTVKIDGVHLGQAVHVSGPIFVLKWNPRNYSSGTHNIEVIVQDSAGRSKSVHHIFSVQENNHLSFDPLASFILRTDHYIMARVLFVLIVLSQLTILIIFRYRGYPELKEPSGFINLTSFSLHVLSKINIFYYSVLLLTLYTVLGPWFFGEIIDGKFGCCFSFGIFVNGHFLQGSITFIIGILQLAFFNIPLMAYMCWSLLQRCFGHNFRSHLHQRKYLKIMPVHLLMLLLYIWQVYSCYFLYATYGTLAFLFSPLRTWLTLLTPVLIRYVWTLNSTKFGIFMVQLKSHLSS

SEQ ID NO: 6 - protein, 806 aa, Homo sapiens
MQFFGRLVNTFSGVTNLFSNPFRVKEVAVADYTSSDRVREEGQLILFQNTPNRTWDCVLVNPRNSQSGFRLFQLELEADALVNFHQYSSQLLPFYESSPQVLHTEVLQHLTDLIRNHPSWSVAHLAVELGIRECFHHSRIISCANCAENEEGCTPLHLACRKGDGEILVELVQYCHTQMDVTDYKGETVFHYAVQGDNSQVLQLLGRNAVAGLNQVNNQGLTPLHLACQLGKQEMVRVLLLCNARCNIMGPNGYPIHSAMKFSQKGCAEMIISMDSSQIHSKDPRYGASPLHWAKNAEMARMLLKRGCNVNSTSSAGNTALHVAVMRNRFDCAIVLLTHGANADARGEHGNTPLHLAMSKDNVEMIKALIVFGAEVDTPNDFGETPTFLASKIGRLVTRKAILTLLRTVGAEYCFPPIHGVPAEQGSAAPHHPFSLERAQPPPISLNNLELQDLMHISRARKPAFILGSMRDEKRTHDHLLCLDGGGVKGLIIIQLLIAIEKASGVATKDLFDWVAGTSTGGILALAILHSKSMAYMRGMYFRMKDEVFRGSRPYESGPLEEFLKREFGEHTKMTDVRKPKVMLTGTLSDRQPAELHLFRNYDAPETVREPRFNQNVNLRPPAQPSDQLVWRAARSSGAAPTYFRPNGRFLDGGLLANNPTLDAMTEIHEYNQDLIRKGQANKVKKLSIVVSLGTGRSPQVPVTCVDVFRPSNPWELAKTVFGAKELGKMVVDCCTDPDGRAVDRARAWCEMVGIQYFRLNPQLGTDIMLDEVSDTVLVNALWETEVYIYEHREEFQKLIQLLLSP

SEQ ID NO: 7 - protein, 9 aa, Homo sapiens
DETCVLWQD

SEQ ID NO: 8 - protein, 10 aa, Homo sapiens
VLWQDIKDAL

SEQ ID NO: 9 - protein, 9 aa, Homo sapiens
LWQDIKDAL

SEQ ID NO: 10 - protein, 9 aa, Homo sapiens
LAVQGTDPT

SEQ ID NO: 11 - protein, 9 aa, Homo sapiens
PSNGDSSSI

SEQ ID NO: 12 - protein, 10 aa, Homo sapiens
KPSNGDSSSI

SEQ ID NO: 13 - protein, 9 aa, Homo sapiens
NNPSILQPV

SEQ ID NO: 14 - protein, 9 aa, Homo sapiens
REKGNNPSI

SEQ ID NO: 15 - protein, 10 aa, Homo sapiens
REKGNNPSIL

SEQ ID NO: 16 - protein, 9 aa, Homo sapiens
YTVLGPWFF

SEQ ID NO: 17 - protein, 9 aa, Homo sapiens
TLYTVLGPW

SEQ ID NO: 18 - protein, 10 aa, Homo sapiens
TLYTVLGPWF

SEQ ID NO: 19 - protein, 9 aa, Homo sapiens
LYTVLGPWF

SEQ ID NO: 20 - protein, 10 aa, Homo sapiens
LTLYTVLGPW

SEQ ID NO: 21 - protein, 10 aa, Homo sapiens
LYTVLGPWFF

SEQ ID NO: 22 - protein, 10 aa, Homo sapiens
VLGPWFFGEI

SEQ ID NO: 23 - protein, 11 aa, Homo sapiens
TFLASKIGRLV

SEQ ID NO: 24 - protein, 9 aa, Homo sapiens
RLVTRKAIL

SEQ ID NO: 25 - protein, 9 aa, Homo sapiens
FLASKIGRL

SEQ ID NO: 26 - protein, 10 aa, Homo sapiens
SKIGRLVTRK

SEQ ID NO: 27 - protein, 10 aa, Homo sapiens
FLASKIGRLV

SEQ ID NO: 28 - protein, 9 aa, Homo sapiens
LASKIGRLV

SEQ ID NO: 29 - protein, 9 aa, Homo sapiens
KIGRLVTRK

SEQ ID NO: 30 - protein, 14 aa, Artificial Sequence
  Description of Artificial Sequence Synthetic peptide
CAVHEIQGAQKLVF

SEQ ID NO: 31 - protein, 13 aa, Artificial Sequence
  Description of Artificial Sequence Synthetic peptide
CASSFGVSYEQYF

SEQ ID NO: 32 - protein, 14 aa, Artificial Sequence
  Description of Artificial Sequence Synthetic peptide
CAMRPLGGYNKLIF

SEQ ID NO: 33 - protein, 12 aa, Artificial Sequence
  Description of Artificial Sequence Synthetic peptide
CASSQAANEQFF

SEQ ID NO: 34 - protein, 13 aa, Artificial Sequence
  Description of Artificial Sequence Synthetic peptide
CAEEGDRDYKLSF

SEQ ID NO: 35 - protein, 14 aa, Artificial Sequence
  Description of Artificial Sequence Synthetic peptide
CASTGRSGRSEQYF

SEQ ID NO: 36 - protein, 13 aa, Artificial Sequence
  Description of Artificial Sequence Synthetic peptide
CAFMKGRDDKIIF

SEQ ID NO: 37 - protein, 13 aa, Artificial Sequence
  Description of Artificial Sequence Synthetic peptide
CATTLPGDTEAFF

SEQ ID NO: 38 - protein, 13 aa, Artificial Sequence
  Description of Artificial Sequence Synthetic peptide
CATANNAGNMLTF

SEQ ID NO: 39 - protein, 13 aa, Artificial Sequence
  Description of Artificial Sequence Synthetic peptide
CASSLDRHQPQHF

SEQ ID NO: 40 - protein, 15 aa, Artificial Sequence
  Description of Artificial Sequence Synthetic peptide
CALWEGQGGSEKLVF

SEQ ID NO: 41 - protein, 17 aa, Artificial Sequence
  Description of Artificial Sequence Synthetic peptide
CASSLEARAPSGNTIYF

SEQ ID NO: 42 - protein, 15 aa, Artificial Sequence
  Description of Artificial Sequence Synthetic peptide
CAVGAGTGTASKLTF

SEQ ID NO: 43 - protein, 16 aa, Artificial Sequence
  Description of Artificial Sequence Synthetic peptide
CASSLELAGGRDTQYF

SEQ ID NO: 44 - protein, 134 aa, Artificial Sequence
  Description of Artificial Sequence Synthetic polypeptide
MLLLLVPAFQVIFTLGGTRAQSVTQLDSQVPVFEEAPVELRCNYSSSVSVYLFWYVQYPNQGLQLLLKYLSGSTLVESINGFEAEFNKSQTSFHLRKPSVHISDTAEYFCAVHEIQGAQKLVFGQGTRLTINPN

SEQ ID NO: 45 - protein, 132 aa, Artificial Sequence
  Description of Artificial Sequence Synthetic polypeptide
MGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSFGVSYEQYFGPGTRLTVT

SEQ ID NO: 46 - protein, 135 aa, Artificial Sequence
  Description of Artificial Sequence Synthetic polypeptide
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMRPLGGYNKLIFGAGTRLAVHP

SEQ ID NO: 47 - protein, 130 aa, Artificial Sequence
  Description of Artificial Sequence Synthetic polypeptide
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQAANEQFFGPGTRLTVL

SEQ ID NO: 48 - protein, 133 aa, Artificial Sequence
  Description of Artificial Sequence Synthetic polypeptide
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEEGDRDYKLSFGAGTTVTVRAN

SEQ ID NO: 49 - protein, 133 aa, Artificial Sequence
  Description of Artificial Sequence Synthetic polypeptide
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASTGRSGRSEQYFGPGTRLTVT

SEQ ID NO: 50 - protein, 274 aa, Artificial Sequence
  Description of Artificial Sequence Synthetic polypeptide
MLLLLVPAFQVIFTLGGTRAQSVTQLDSQVPVFEEAPVELRCNYSSSVSVYLFWYVQYPNQGLQLLLKYLSGSTLVESINGFEAEFNKSQTSFHLRKPSVHISDTAEYFCAVHEIQGAQKLVFGQGTRLTINPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 51 - protein, 311 aa, Artificial Sequence
  Description of Artificial Sequence Synthetic polypeptide
MGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSFGVSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 52 - protein, 276 aa, Artificial Sequence
  Description of Artificial Sequence Synthetic polypeptide
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMRPLGGYNKLIFGAGTRLAVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 53 - protein, 309 aa, Artificial Sequence
  Description of Artificial Sequence Synthetic polypeptide
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQAANEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 54 - protein, 273 aa, Artificial Sequence
  Description of Artificial Sequence Synthetic polypeptide
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEEGDRDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 55 - protein, 179 aa, Artificial Sequence
  Description of Artificial Sequence Synthetic polypeptide
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 56 - protein, 141 aa, Artificial Sequence
  Description of Artificial Sequence Synthetic polypeptide
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 57 - protein, 178 aa, Artificial Sequence
  Description of Artificial Sequence Synthetic polypeptide
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 58 - protein, 140 aa, Artificial Sequence
  Description of Artificial Sequence Synthetic polypeptide
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 59 - protein, 179 aa, Artificial Sequence; identical to SEQ ID NO: 55
  Description of Artificial Sequence Synthetic polypeptide

SEQ ID NO: 60 - protein, 140 aa, Artificial Sequence; identical to SEQ ID NO: 58
  Description of Artificial Sequence Synthetic polypeptide

SEQ ID NO: 61 - protein, 179 aa, Artificial Sequence; identical to SEQ ID NO: 55
  Description of Artificial Sequence Synthetic polypeptide

SEQ ID NO: 62 - protein, 9 aa, Homo sapiens
YAIVWVNGV

SEQ ID NO: 63 - protein, 9 aa, Homo sapiens
AAYGRSPMV

SEQ ID NO: 64 - protein, 9 aa, Homo sapiens
AFYDPTHAW

SEQ ID NO: 65 - protein, 9 aa, Homo sapiens
AGAEGFVRV

SEQ ID NO: 66 - protein, 9 aa, Homo sapiens
ALIEKLVEL

SEQ ID NO: 67 - protein, 10 aa, Homo sapiens
ALVTPGSLGL

SEQ ID NO: 68 - protein, 10 aa, Homo sapiens
AQAEDMNVKL

SEQ ID NO: 69 - protein, 9 aa, Homo sapiens
AQIDHYLGL

SEQ ID NO: 70 - protein, 9 aa, Homo sapiens
AQLELISRL

SEQ ID NO: 71 - protein, 9 aa, Homo sapiens
AQVHHFMEL

SEQ ID NO: 72 - protein, 9 aa, Homo sapiens
ATDDSLHQL

SEQ ID NO: 73 - protein, 9 aa, Homo sapiens
ATFKGIVRA

SEQ ID NO: 74 - protein, 9 aa, Homo sapiens
ATLGNFAKA

SEQ ID NO: 75 - protein, 9 aa, Homo sapiens
ATYVFLHTV

SEQ ID NO: 76 - protein, 9 aa, Homo sapiens
AVDFGNYHL

SEQ ID NO: 77 - protein, 9 aa, Homo sapiens
AVDLTLPKV

SEQ ID NO: 78 - protein, 9 aa, Homo sapiens
AVFKELVKL

SEQ ID NO: 79 - protein, 9 aa, Homo sapiens
AVIKGLTYL

SEQ ID NO: 80 - protein, 9 aa, Homo sapiens
AVLDRMHSL

SEQ ID NO: 81 - protein, 9 aa, Homo sapiens
AVQEKVFEL

SEQ ID NO: 82 - protein, 9 aa, Homo sapiens
AVVTQVNKV

SEQ ID NO: 83 - protein, 9 aa, Homo sapiens
AYQYTIRAH

SEQ ID NO: 84 - protein, 9 aa, Homo sapiens
AYYVSNEEL

SEQ ID NO: 85 - protein, 9 aa, Homo sapiens
DYQALRTSI

SEQ ID NO: 86 - protein, 9 aa, Homo sapiens
ELFTGFIKV

SEQ ID NO: 87 - protein, 9 aa, Homo sapiens
EQIAEFKEA

SEQ ID NO: 88 - protein, 9 aa, Homo sapiens
EQIGQTHRV

SEQ ID NO: 89 - protein, 9 aa, Homo sapiens
EQIKDIFYI

SEQ ID NO: 90 - protein, 9 aa, Homo sapiens
EVIEKFDYV

SEQ ID NO: 91 - protein, 9 aa, Homo sapiens
EVPEYLNHV

SEQ ID NO: 92 - protein, 9 aa, Homo sapiens
EYVKANDAY

SEQ ID NO: 93 - protein, 9 aa, Homo sapiens
FAMEIDPSL

SEQ ID NO: 94 - protein, 9 aa, Homo sapiens
FASHVSPEV

SEQ ID NO: 95 - protein, 9 aa, Homo sapiens
FILEKIEYL

SEQ ID NO: 96 - protein, 9 aa, Homo sapiens
FISEFEHRV

SEQ ID NO: 97 - protein, 9 aa, Homo sapiens
FLFNKVVNL

SEQ ID NO: 98 - protein, 9 aa, Homo sapiens
FLKNELDNV

SEQ ID NO: 99 - protein, 9 aa, Homo sapiens
FLLEKGYEV

SEQ ID NO: 100 - protein, 9 aa, Homo sapiens
FLMDFIHQV

SEQ ID NO: 101 - protein, 9 aa, Homo sapiens
FPHHLQEYI

SEQ ID NO: 102 - protein, 9 aa, Homo sapiens
FPRHIEPEI

SEQ ID NO: 103 - protein, 9 aa, Homo sapiens
FPYMTHPSV

SEQ ID NO: 104 - protein, 9 aa, Homo sapiens
FQDGTKATF

SEQ ID NO: 105 - protein, 9 aa, Homo sapiens
FSLEELRVA

SEQ ID NO: 106 - protein, 9 aa, Homo sapiens
FSSPHLVQV

SEQ ID NO: 107 - protein, 9 aa, Homo sapiens
FTVPRVVAV

SEQ ID NO: 108 - protein, 9 aa, Homo sapiens
FVLDKVPFL

SEQ ID NO: 109 - protein, 9 aa, Homo sapiens
FVLPVTPPR

SEQ ID NO: 110 - protein, 9 aa, Homo sapiens
FVPIKVEQI

SEQ ID NO: 111 - protein, 10 aa, Homo sapiens
FYYDGKVMKL

SEQ ID NO: 112 - protein, 9 aa, Homo sapiens
GIMDRFLQV

SEQ ID NO: 113 - protein, 10 aa, Homo sapiens
GLDVDGIYRV

SEQ ID NO: 114 - protein, 9 aa, Homo sapiens
GMPDFLEKL

SEQ ID NO: 115 - protein, 9 aa, Homo sapiens
GMYIFLHTV

SEQ ID NO: 116 - protein, 9 aa, Homo sapiens
GQDVGRYQV

SEQ ID NO: 117 - protein, 9 aa, Homo sapiens
GQIDSMEQL

SEQ ID NO: 118 - protein, 9 aa, Homo sapiens
GQLDKVVEL

SEQ ID NO: 119 - protein, 9 aa, Homo sapiens
GQLISVEKV

SEQ ID NO: 120 - protein, 9 aa, Homo sapiens
GQLTKLHQL

SEQ ID NO: 121 - protein, 9 aa, Homo sapiens
GQTGKLMYV

SEQ ID NO: 122 - protein, 9 aa, Homo sapiens
GQTPVFSKA

SEQ ID NO: 123 - protein, 11 aa, Homo sapiens
GVAESIHLWEV

SEQ ID NO: 124 - protein, 10 aa, Homo sapiens
GVIAEILRGV

SEQ ID NO: 125 - protein, 9 aa, Homo sapiens
GVIKRTENV

SEQ ID NO: 126 - protein, 9 aa, Homo sapiens
GVYGDVHRV

SEQ ID NO: 127 - protein, 9 aa, Homo sapiens
GVYGDVQRV

SEQ ID NO: 128 - protein, 9 aa, Homo sapiens
HAAELIKTV

SEQ ID NO: 129 - protein, 9 aa, Homo sapiens
HAHPFFFTI

SEQ ID NO: 130 - protein, 9 aa, Homo sapiens
HAIPSMSHV

SEQ ID NO: 131 - protein, 9 aa, Homo sapiens
HFFDGKTAL

SEQ ID NO: 132 - protein, 9 aa, Homo sapiens
HLIGDFSKV

SEQ ID NO: 133 - protein, 9 aa, Homo sapiens
HLIHEVTKV

SEQ ID NO: 134 - protein, 10 aa, Homo sapiens
HQLENLSNFI

SEQ ID NO: 135 - protein, 9 aa, Homo sapiens
HSIPVTVEV

SEQ ID NO: 136 - protein, 9 aa, Homo sapiens
HVADMLVKV

SEQ ID NO: 137 - protein, 10 aa, Homo sapiens
HVFDHPWETV

SEQ ID NO: 138 - protein, 9 aa, Homo sapiens
IIDQHTFEV

SEQ ID NO: 139 - protein, 9 aa, Homo sapiens
IPPIQVTKV

SEQ ID NO: 140 - protein, 9 aa, Homo sapiens
IQDDMHLVI

SEQ ID NO: 141 - protein, 9 aa, Homo sapiens
IQKESTLHL

SEQ ID NO: 142 - protein, 9 aa, Homo sapiens
IQVRETQAL

SEQ ID NO: 143 - protein, 9 aa, Homo sapiens
IQVTKVTQV

SEQ ID NO: 144 - protein, 9 aa, Homo sapiens
ISFTEFVKV

SEQ ID NO: 145 - protein, 9 aa, Homo sapiens
ITFEDFLKI

SEQ ID NO: 146 - protein, 9 aa, Homo sapiens
IYQIHKEYL

SEQ ID NO: 147 - protein, 9 aa, Homo sapiens
KIAEIEAEM

SEQ ID NO: 148 - protein, 9 aa, Homo sapiens
KILPTLEAV

SEQ ID NO: 149 - protein, 9 aa, Homo sapiens
KILQELPSV

SEQ ID NO: 150 - protein, 9 aa, Homo sapiens
KLIDRTESL

SEQ ID NO: 151 - protein, 9 aa, Homo sapiens
KLILLITQV

SEQ ID NO: 152 - protein, 9 aa, Homo sapiens
KLISDINKA

SEQ ID NO: 153 - protein, 9 aa, Homo sapiens
KLISELQKL

SEQ ID NO: 154 - protein, 10 aa, Homo sapiens
KLLDPEDISV

SEQ ID NO: 155 - protein, 9 aa, Homo sapiens
KLPEKWESV

SEQ ID NO: 156 - protein, 9 aa, Homo sapiens
KLVGEFLEV

SEQ ID NO: 157 - protein, 9 aa, Homo sapiens
KMPEINAKV

SEQ ID NO: 158 - protein, 9 aa, Homo sapiens
KPYAHILSV

SEQ ID NO: 159 - protein, 9 aa, Homo sapiens
KQADPFASL

SEQ ID NO: 160 - protein, 9 aa, Homo sapiens
KQAEIVKRL

SEQ ID NO: 161 - protein, 9 aa, Homo sapiens
KQFEKQVEL

SEQ ID NO: 162 - protein, 9 aa, Homo sapiens
KQLEGIWHT

SEQ ID NO: 163 - protein, 9 aa, Homo sapiens
KQMEDGHTL

SEQ ID NO: 164 - protein, 9 aa, Homo sapiens
KQTDLEITV

SEQ ID NO: 165 - protein, 9 aa, Homo sapiens
KTLERSYLL

SEQ ID NO: 166 - protein, 10 aa, Homo sapiens
KTVEGEFTGV

SEQ ID NO: 167 - protein, 9 aa, Homo sapiens
KVIDEIYRV

SEQ ID NO: 168 - protein, 9 aa, Homo sapiens
KVIDGLETL

SEQ ID NO: 169 - protein, 9 aa, Homo sapiens
KVLAAVYKA

SEQ ID NO: 170 - protein, 9 aa, Homo sapiens
KVLDILQHI

SEQ ID NO: 171 - protein, 9 aa, Homo sapiens
KVLGIVVGV

SEQ ID NO: 172 - protein, 10 aa, Homo sapiens
KVLGQGKWNL

SEQ ID NO: 173 - protein, 9 aa, Homo sapiens
KVLMEWINA

SEQ ID NO: 174 - protein, 9 aa, Homo sapiens
KVTNEFVHI

SEQ ID NO: 175 - protein, 9 aa, Homo sapiens
KYFPSRVSI

SEQ ID NO: 176 - protein, 9 aa, Homo sapiens
KYHVSEKPY

SEQ ID NO: 177 - protein, 9 aa, Homo sapiens
KYMEHRQTY

SEQ ID NO: 178 - protein, 9 aa, Homo sapiens
LAPTDVKEL

SEQ ID NO: 179 - protein, 9 aa, Homo sapiens
LILDPIFKV

SEQ ID NO: 180 - protein, 9 aa, Homo sapiens
LLDEEISRV

SEQ ID NO: 181 - protein, 10 aa, Homo sapiens
LLFEGEKITI

SEQ ID NO: 182 - protein, 9 aa, Homo sapiens
LPFEPVPVI

SEQ ID NO: 183 - protein, 9 aa, Homo sapiens
LPKEPQGII

SEQ ID NO: 184 - protein, 9 aa, Homo sapiens
LPLLEQKQV

SEQ ID NO: 185 - protein, 9 aa, Homo sapiens
LPPNLYPTV

SEQ ID NO: 186 - protein, 9 aa, Homo sapiens
LPRAEVSRI

SEQ ID NO: 187 - protein, 9 aa, Homo sapiens
LQTSIREEI

SEQ ID NO: 188 - protein, 9 aa, Homo sapiens
LYATVHQSY

SEQ ID NO: 189 - protein, 9 aa, Homo sapiens
LYQKEVSHL

SEQ ID NO: 190 - protein, 9 aa, Homo sapiens
MPDDHARKI

SEQ ID NO: 191 - protein, 9 aa, Homo sapiens
MPLVKSVTV

SEQ ID NO: 192 - protein, 9 aa, Homo sapiens
MPPLNTQRI

SEQ ID NO: 193 - protein, 10 aa, Homo sapiens
MQAERIFSEV

SEQ ID NO: 194 - protein, 9 aa, Homo sapiens
NALRELETV

SEQ ID NO: 195 - protein, 9 aa, Homo sapiens
NAMPVRLQV

SEQ ID NO: 196 - protein, 9 aa, Homo sapiens
NFQAIRVTL

SEQ ID NO: 197 - protein, 9 aa, Homo sapiens
NFSHGTHEY

SEQ ID NO: 198 - protein, 9 aa, Homo sapiens
NMVDIIHSV

SEQ ID NO: 199 - protein, 9 aa, Homo sapiens
NQADTTHPL

SEQ ID NO: 200 - protein, 9 aa, Homo sapiens
NQFDVVIRL

SEQ ID NO: 201 - protein, 10 aa, Homo sapiens
NQIGAKFWEV

SEQ ID NO: 202 - protein, 9 aa, Homo sapiens
NTFTKLITV

SEQ ID NO: 203 - protein, 9 aa, Homo sapiens
NVIERLQEA

SEQ ID NO: 204 - protein, 9 aa, Homo sapiens
NVPDSFNEV

SEQ ID NO: 205 - protein, 9 aa, Homo sapiens
QIGAKFWEV

SEQ ID NO: 206 - protein, 9 aa, Homo sapiens
QMISRIEYI

SEQ ID NO: 207 - protein, 9 aa, Homo sapiens
QMYAKYVKM

SEQ ID NO: 208 - protein, 9 aa, Homo sapiens
QQIGEKIFA

SEQ ID NO: 209 - protein, 9 aa, Homo sapiens
QTPQRLLEV

SEQ ID NO: 210 - protein, 9 aa, Homo sapiens
QVIEELFKA

SEQ ID NO: 211 - protein, 9 aa, Homo sapiens
QVLEDFPTI

SEQ ID NO: 212 - protein, 9 aa, Homo sapiens
QVMDEVIKV

SEQ ID NO: 213 - protein, 10 aa, Homo sapiens
QYYNGKIHAY

SEQ ID NO: 214 - protein, 9 aa, Homo sapiens
RIPIAVSGV

SEQ ID NO: 215 - protein, 10 aa, Homo sapiens
RLLDEEISRV

SEQ ID NO: 216 - protein, 9 aa, Homo sapiens
RQDLTTLDV

SEQ ID NO: 217 - protein, 9 aa, Homo sapiens
RVIGTLEEV

SEQ ID NO: 218 - protein, 9 aa, Homo sapiens
SALKEFEQV

SEQ ID NO: 219 - protein, 9 aa, Homo sapiens
SFLPSGSEM

SEQ ID NO: 220 - protein, 9 aa, Homo sapiens
SFQDFVSHF

SEQ ID NO: 221 - protein, 9 aa, Homo sapiens
SFSEQTRSL

SEQ ID NO: 222 - protein, 9 aa, Homo sapiens
SIIGRLLEV

SEQ ID NO: 223 - protein, 9 aa, Homo sapiens
SIIQRLLEV

SEQ ID NO: 224 - protein, 9 aa, Homo sapiens
SLITRLLEV

SEQ ID NO: 225 - protein, 9 aa, Homo sapiens
SLYKSFLQL

SEQ ID NO: 226 - protein, 9 aa, Homo sapiens
SQADKFLSL

SEQ ID NO: 227 - protein, 9 aa, Homo sapiens
SQDHFHVFV

SEQ ID NO: 228 - protein, 9 aa, Homo sapiens
SQDPGTHVV

SEQ ID NO: 229 - protein, 10 aa, Homo sapiens
SQFDEELYKV

SEQ ID NO: 230 - protein, 9 aa, Homo sapiens
SQFNKEILL

SEQ ID NO: 231 - protein, 9 aa, Homo sapiens
SQSTVKVLI

SEQ ID NO: 232 - protein, 9 aa, Homo sapiens
STVKVLIRL

SEQ ID NO: 233 - protein, 9 aa, Homo sapiens
SVAEKLSKL

SEQ ID NO: 234 - protein, 9 aa, Homo sapiens
SVASSVKGV

SEQ ID NO: 235 - protein, 9 aa, Homo sapiens
SVIKIFPDL

SEQ ID NO: 236 - protein, 9 aa, Homo sapiens
SVLKELLQV

SEQ ID NO: 237 - protein, 9 aa, Homo sapiens
SVVDHFNRI

SEQ ID NO: 238 - protein, 9 aa, Homo sapiens
SYIIGTSSV

SEQ ID NO: 239 - protein, 9 aa, Homo sapiens
SYKKPGIHL

SEQ ID NO: 240 - protein, 9 aa, Homo sapiens
TAISLFYEL

SEQ ID NO: 241 - protein, 9 aa, Homo sapiens
TILTEIYRI

SEQ ID NO: 242 - protein, 9 aa, Homo sapiens
TIQEEFYRV

SEQ ID NO: 243 - protein, 10 aa, Homo sapiens
TLFEDSFQQI

SEQ ID NO: 244 - protein, 9 aa, Homo sapiens
TLPDLVPQL

SEQ ID NO: 245 - protein, 9 aa, Homo sapiens
TPYWMAPEV

SEQ ID NO: 246 - protein, 9 aa, Homo sapiens
TQIDHYVGI

SEQ ID NO: 247 - protein, 9 aa, Homo sapiens
TQVTALIKL

SEQ ID NO: 248 - protein, 9 aa, Homo sapiens
TVADHFDHI

SEQ ID NO: 249 - protein, 9 aa, Homo sapiens
TVAEHLWKL

SEQ ID NO: 250 - protein, 9 aa, Homo sapiens
TVAKELQTL

SEQ ID NO: 251 - protein, 9 aa, Homo sapiens
TVLDKTLSV

SEQ ID NO: 252 - protein, 9 aa, Homo sapiens
TVVERLVKV

SEQ ID NO: 253 - protein, 9 aa, Homo sapiens
TVVMYLHHV

SEQ ID NO: 254 - protein, 9 aa, Homo sapiens
TYGERVVAF

SEQ ID NO: 255 - protein, 9 aa, Homo sapiens
TYSEFAREL

SEQ ID NO: 256 - protein, 9 aa, Homo sapiens
VIAEILRGV

SEQ ID NO: 257 - protein, 9 aa, Homo sapiens
VILKILPTL

SEQ ID NO: 258 - protein, 9 aa, Homo sapiens
VLLGKVYVV

SEQ ID NO: 259 - protein, 9 aa, Homo sapiens
VLVDQTTGL

SEQ ID NO: 260 - protein, 9 aa, Homo sapiens
VPAQVQSEI

SEQ ID NO: 261 - protein, 9 aa, Homo sapiens
VQIEEVRQV

SEQ ID NO: 262 - protein, 9 aa, Homo sapiens
VQPEAQAKV

SEQ ID NO: 263 - protein, 9 aa, Homo sapiens
VSPVSFHQV

SEQ ID NO: 264 - protein, 9 aa, Homo sapiens
VTASSLVKI

SEQ ID NO: 265 - protein, 9 aa, Homo sapiens
VTLEKVLGI

SEQ ID NO: 266 - protein, 9 aa, Homo sapiens
VVDEKLPGL

SEQ ID NO: 267 - protein, 9 aa, Homo sapiens
VVLEEIPKL

SEQ ID NO: 268 - protein, 9 aa, Homo sapiens
VVLPKNVNL

SEQ ID NO: 269 - protein, 9 aa, Homo sapiens
VYIPSKTDL

SEQ ID NO: 270 - protein, 9 aa, Homo sapiens
VYSPHVLNL

SEQ ID NO: 271 - protein, 9 aa, Homo sapiens
WPYGNRQEI

SEQ ID NO: 272 - protein, 9 aa, Homo sapiens
YAIEKIERI

SEQ ID NO: 273 - protein, 9 aa, Homo sapiens
YAVELFEGL

SEQ ID NO: 274 - protein, 9 aa, Homo sapiens
YFAERVTSL

SEQ ID NO: 275 - protein, 10 aa, Homo sapiens
YFIPATGHSL

SEQ ID NO: 276 - protein, 9 aa, Homo sapiens
YFIVTRQDY

SEQ ID NO: 277 - protein, 9 aa, Homo sapiens
YIPTELDQV

SEQ ID NO: 278 - protein, 9 aa, Homo sapiens
YISEENFRV

SEQ ID NO: 279 - protein, 9 aa, Homo sapiens
YLTHDSPSV

SEQ ID NO: 280 - protein, 9 aa, Homo sapiens
YPESNSRAI

SEQ ID NO: 281 - protein, 9 aa, Homo sapiens
YQDPQGRHV

SEQ ID NO: 282 - protein, 9 aa, Homo sapiens
YQLSKEYTL

SEQ ID NO: 283 - protein, 9 aa, Homo sapiens
YVMDTRPKL

SEQ ID NO: 284 - protein, 9 aa, Homo sapiens
YVTEKVLAA

SEQ ID NO: 285 - protein, 9 aa, Homo sapiens
YYFDYKEQL

SEQ ID NO: 286 - protein, 9 aa, Homo sapiens
YYNGKIHAY

SEQ ID NO: 287 - protein, 10 aa, Homo sapiens
YYQTAPKAGY

SEQ ID NO: 288 - protein, 10 aa, Homo sapiens
YYRYPTGESY

SEQ ID NO: 289 - protein, 10 aa, Homo sapiens
YYYDGDIGNY

SEQ ID NO: 290 - protein, 11 aa, Homo sapiens
YYYDGDIGNYY

SEQ ID NO: 291 - protein, 9 aa, Homo sapiens
YYYNGKAVY

SEQ ID NO: 292 - protein, 9 aa, Homo sapiens
YYYTKEEQF

SEQ ID NO: 293 - protein, 9 aa, Homo sapiens
AASTARHLY

SEQ ID NO: 294 - protein, 9 aa, Homo sapiens
AEAYLGKTV

SEQ ID NO: 295 - protein, 9 aa, Homo sapiens
AEDELFNRY

SEQ ID NO: 296 - protein, 9 aa, Homo sapiens
AEDYAKERY

SEQ ID NO: 297 - protein, 9 aa, Homo sapiens
AEEESFRKL

SEQ ID NO: 298 - protein, 9 aa, Homo sapiens
AEEKQTKSF

SEQ ID NO: 299 - protein, 9 aa, Homo sapiens
AEELERQGY

SEQ ID NO: 300 - protein, 9 aa, Homo sapiens
AEHDVTVKY

SEQ ID NO: 301 - protein, 10 aa, Homo sapiens
AEHDVTVKYY

SEQ ID NO: 302 - protein, 9 aa, Homo sapiens
AEHIESRTL

SEQ ID NO: 303 - protein, 10 aa, Homo sapiens
AEHPNPGVRY

SEQ ID NO: 304 - protein, 10 aa, Homo sapiens
AEITPKGRAL

SEQ ID NO: 305 - protein, 9 aa, Homo sapiens
AEKELHEKF

SEQ ID NO: 306 - protein, 9 aa, Homo sapiens
AEKLITQTF

SEQ ID NO: 307 - protein, 10 aa, Homo sapiens
AELESSKEQF

SEQ ID NO: 308 - protein, 10 aa, Homo sapiens
AELPAELTGV

SEQ ID NO: 309 - protein, 9 aa, Homo sapiens
AELTPHQTF

SEQ ID NO: 310 - protein, 10 aa, Homo sapiens
AELYINEHDY

SEQ ID NO: 311 - protein, 9 aa, Homo sapiens
AEMQWKRDY

SEQ ID NO: 312 - protein, 9 aa, Homo sapiens
AENLGKEHF

SEQ ID NO: 313 - protein, 10 aa, Homo sapiens
AETGNLPHSY

SEQ ID NO: 314 - protein, 10 aa, Homo sapiens
AEVDKVTGRF

SEQ ID NO: 315 - protein, 9 aa, Homo sapiens
AEVEPGVHL

SEQ ID NO: 316 - protein, 10 aa, Homo sapiens
AEVNSSGKTI

SEQ ID NO: 317 - protein, 10 aa, Homo sapiens
AEYNSDLHQF

SEQ ID NO: 318 - protein, 9 aa, Homo sapiens
AFQERLNSY

SEQ ID NO: 319 - protein, 10 aa, Homo sapiens
DEINHVQKTY

SEQ ID NO: 320 - protein, 10 aa, Homo sapiens
DEVQVVRGHY

SEQ ID NO: 321 - protein, 9 aa, Homo sapiens
EEAERPLHF

SEQ ID NO: 322 - protein, 10 aa, Homo sapiens
EEAQFETKKL

SEQ ID NO: 323 - protein, 10 aa, Homo sapiens
EEAQFETKKY

SEQ ID NO: 324 - protein, 9 aa, Homo sapiens
EEAQYQREY

SEQ ID NO: 325 - protein, 10 aa, Homo sapiens
EEAYNAVVRY

SEQ ID NO: 326 - protein, 10 aa, Homo sapiens
EEDAALFKAW

SEQ ID NO: 327 - protein, 9 aa, Homo sapiens
EEEKGGKVY

SEQ ID NO: 328 - protein, 10 aa, Homo sapiens
EEEPLNIHSF

SEQ ID NO: 329 - protein, 10 aa, Homo sapiens
EEEQAVRPKY

SEQ ID NO: 330 - protein, 10 aa, Homo sapiens
EEEQENNKSF

SEQ ID NO: 331 - protein, 10 aa, Homo sapiens
EEFLNGRAVY

SEQ ID NO: 332 - protein, 9 aa, Homo sapiens
EEGLVNNKY

SEQ ID NO: 333 - protein, 10 aa, Homo sapiens
EEGQLRPKTY

SEQ ID NO: 334 - protein, 9 aa, Homo sapiens
EEIKKNYRY

SEQ ID NO: 335 - protein, 10 aa, Homo sapiens
EEIVYPQKAY

SEQ ID NO: 336 - protein, 11 aa, Homo sapiens
EEIVYPQKAYY

SEQ ID NO: 337 - protein, 9 aa, Homo sapiens
EELYTKKLW

SEQ ID NO: 338 - protein, 9 aa, Homo sapiens
EEPTVIKKY

SEQ ID NO: 339 - protein, 9 aa, Homo sapiens
EEQAVRPKY

SEQ ID NO: 340 - protein, 9 aa, Homo sapiens
EETELDRMW

SEQ ID NO: 341 - protein, 10 aa, Homo sapiens
EETNIPKRLY

SEQ ID NO: 342 - protein, 10 aa, Homo sapiens
EEVEAKFKRY

SEQ ID NO: 343 - protein, 10 aa, Homo sapiens
EEVPVYENKY

SEQ ID NO: 344 - protein, 10 aa, Homo sapiens
EEVRQGLKAY

SEQ ID NO: 345 - protein, 10 aa, Homo sapiens
FEYNENTRKY

SEQ ID NO: 346 - protein, 10 aa, Homo sapiens
FFPDKPITQY

SEQ ID NO: 347 - protein, 9 aa, Homo sapiens
FISEFEHRV

SEQ ID NO: 348 - protein, 9 aa, Homo sapiens
FLTTLKPRL

SEQ ID NO: 349 - protein, 9 aa, Homo sapiens
FLYAGHIFL

SEQ ID NO: 350 - protein, 10 aa, Homo sapiens
GEITHPKNNY

SEQ ID NO: 351 - protein, 9 aa, Homo sapiens
GELLPTKKF

SEQ ID NO: 352 - protein, 10 aa, Homo sapiens
GENGTGKTTF

SEQ ID NO: 353 - protein, 10 aa, Homo sapiens
GEWTPTLQHY

SEQ ID NO: 354 - protein, 9 aa, Homo sapiens
GTSSVARAW

SEQ ID NO: 355 - protein, 10 aa, Homo sapiens
HEQEAILHNF

SEQ ID NO: 356 - protein, 10 aa, Homo sapiens
HESDINSVRY

SEQ ID NO: 357 - protein, 9 aa, Homo sapiens
HLAVVAPRL

SEQ ID NO: 358 - protein, 10 aa, Homo sapiens
HLIDTNKIQL

SEQ ID NO: 359 - protein, 11 aa, Homo sapiens
HSNEQTLQRSW

SEQ ID NO: 360 - protein, 9 aa, Homo sapiens
IEFARKNVY

SEQ ID NO: 361 - protein, 9 aa, Homo sapiens
IENPALLRW

SEQ ID NO: 362 - protein, 9 aa, Homo sapiens
IENQNSRQY

SEQ ID NO: 363 - protein, 10 aa, Homo sapiens
IGTSSVARAW

SEQ ID NO: 364 - protein, 9 aa, Homo sapiens
ILDAGGHNV

SEQ ID NO: 365 - protein, 9 aa, Homo sapiens
ILDKKVEKV

SEQ ID NO: 366 - protein, 9 aa, Homo sapiens
ILQEREYRL

SEQ ID NO: 367 - protein, 9 aa, Homo sapiens
ITAPTVRQY

SEQ ID NO: 368 - protein, 9 aa, Homo sapiens
ITQGIPRSY

SEQ ID NO: 369 - protein, 9 aa, Homo sapiens
KAASLIQGY

SEQ ID NO: 370 - protein, 10 aa, Homo sapiens
KAWNDIKKVY

SEQ ID NO: 371 - protein, 9 aa, Homo sapiens
KEAETRAEF

SEQ ID NO: 372 - protein, 10 aa, Homo sapiens
KEIDMLLKEY

SEQ ID NO: 373 - protein, 9 aa, Homo sapiens
KEIGTKQSF

SEQ ID NO: 374 - protein, 9 aa, Homo sapiens
KELELAHRY

SEQ ID NO: 375 - protein, 10 aa, Homo sapiens
KELESIKETY

SEQ ID NO: 376 - protein, 9 aa, Homo sapiens
KESIAQRSY

SEQ ID NO: 377 - protein, 9 aa, Homo sapiens
KESTLHLVL

SEQ ID NO: 378 - protein, 9 aa, Homo sapiens
KEYDLAKKY

SEQ ID NO: 379 - protein, 9 aa, Homo sapiens
KEYEKALKY

SEQ ID NO: 380 - protein, 9 aa, Homo sapiens
KLFEKVKEV

SEQ ID NO: 381 - protein, 9 aa, Homo sapiens
KLLTEVHAA

SEQ ID NO: 382 - protein, 9 aa, Homo sapiens
KMADIEYRL

SEQ ID NO: 383 - protein, 10 aa, Homo sapiens
KMMNGGHYTY

SEQ ID NO: 384 - protein, 9 aa, Homo sapiens
KSMETKVQF

SEQ ID NO: 385 - protein, 9 aa, Homo sapiens
KVIDEIYRV

SEQ ID NO: 386 - protein, 9 aa, Homo sapiens
KVQEQVHKV

SEQ ID NO: 387 - protein, 10 aa, Homo sapiens
LEELYTKKLW

SEQ ID NO: 388 - protein, 9 aa, Homo sapiens
LEVDLKNGY

SEQ ID NO: 389 - protein, 9 aa, Homo sapiens
LLANKVPAA

SEQ ID NO: 390 - protein, 9 aa, Homo sapiens
LLDEEISRV

SEQ ID NO: 391 - protein, 9 aa, Homo sapiens
METHIMHTF

SEQ ID NO: 392 - protein, 10 aa, Homo sapiens
MEVNDIKKAL

SEQ ID NO: 393 - protein, 9 aa, Homo sapiens
MVPPEWHRW

SEQ ID NO: 394 - protein, 9 aa, Homo sapiens
NEDLIKHGW

SEQ ID NO: 395 - protein, 9 aa, Homo sapiens
NEGDKYKFF

SEQ ID NO: 396 - protein, 10 aa, Homo sapiens
NEHDYAKAAF

SEQ ID NO: 397 - protein, 10 aa, Homo sapiens
NEIEDTFRQF

SEQ ID NO: 398 - protein, 10 aa, Homo sapiens
NELDAVQREY

SEQ ID NO: 399 - protein, 9 aa, Homo sapiens
NEQPRQLFW

SEQ ID NO: 400 - protein, 9 aa, Homo sapiens
NEQTLQRSW

SEQ ID NO: 401 - protein, 9 aa, Homo sapiens
QEAGIKTAF

SEQ ID NO: 402 - protein, 10 aa, Homo sapiens
QEAVHSAKEY

SEQ ID NO: 403 - protein, 9 aa, Homo sapiens
QEDEVMRAY

SEQ ID NO: 404 - protein, 9 aa, Homo sapiens
QEDLRTFSW

SEQ ID NO: 405 - protein, 10 aa, Homo sapiens
QEDRPGLHAY

SEQ ID NO: 406 - protein, 11 aa, Homo sapiens
QEFEKHEVHQY

SEQ ID NO: 407 - protein, 10 aa, Homo sapiens
QEFLKPSDSF

SEQ ID NO: 408 - protein, 9 aa, Homo sapiens
QEGDTTRKY

SEQ ID NO: 409 - protein, 9 aa, Homo sapiens
QEHVKSFSW

SEQ ID NO: 410 - protein, 10 aa, Homo sapiens
QENNTKQLRF

SEQ ID NO: 411 - protein, 10 aa, Homo sapiens
QESAPVHNSF

SEQ ID NO: 412 - protein, 9 aa, Homo sapiens
QEVDFFQRY

SEQ ID NO: 413 - protein, 10 aa, Homo sapiens
QEYQKALQEY

SEQ ID NO: 414 - protein, 9 aa, Homo sapiens
QLAEDLKKV

SEQ ID NO: 415 - protein, 9 aa, Homo sapiens
QMISRIEYV

SEQ ID NO: 416 - protein, 9 aa, Homo sapiens
QTFRLYREY

SEQ ID NO: 417 - protein, 9 aa, Homo sapiens
QTLQRSWAY

SEQ ID NO: 418 - protein, 11 aa, Homo sapiens
RGAEYVSAREW

SEQ ID NO: 419 - protein, 10 aa, Homo sapiens
RLLDEEISRV

SEQ ID NO: 420 - protein, 9 aa, Homo sapiens
RVADRLYGV

SEQ ID NO: 421 - protein, 10 aa, Homo sapiens
SEAQEGLQKF

SEQ ID NO: 422 - protein, 10 aa, Homo sapiens
SEEEKGGKVY

SEQ ID NO: 423 - protein, 10 aa, Homo sapiens
SEEGHPFKAY

SEQ ID NO: 424 - protein, 9 aa, Homo sapiens
SEFPGAQHY

SEQ ID NO: 425 - protein, 10 aa, Homo sapiens
SEGDITQKGY

SEQ ID NO: 426 - protein, 9 aa, Homo sapiens
SEGFSHSRW

SEQ ID NO: 427 - protein, 10 aa, Homo sapiens
SEGKITGRRY

SEQ ID NO: 428 - protein, 9 aa, Homo sapiens
SEGYPGKRY

SEQ ID NO: 429 - protein, 10 aa, Homo sapiens
SEGYPGKRYY

SEQ ID NO: 430 - protein, 9 aa, Homo sapiens
SEGYPGQRY

SEQ ID NO: 431 - protein, 9 aa, Homo sapiens
SEHAVNKQL

SEQ ID NO: 432 - protein, 9 aa, Homo sapiens
SELQSQVRY

SEQ ID NO: 433 - protein, 9 aa, Homo sapiens
SENELKKAY

SEQ ID NO: 434 - protein, 10 aa, Homo sapiens
SENELKKAYR

SEQ ID NO: 435 - protein, 9 aa, Homo sapiens
SEVDLTRSF

SEQ ID NO: 436 - protein, 9 aa, Homo sapiens
SEVPFSKRY

SEQ ID NO: 437 - protein, 10 aa, Homo sapiens
SEVPFSKRYL

SEQ ID NO: 438 - protein, 10 aa, Homo sapiens
SEVTASSRHY

SEQ ID NO: 439 - protein, 10 aa, Homo sapiens
SFYEKGPLTF

SEQ ID NO: 440 - protein, 9 aa, Homo sapiens
SLDGRQIRV

SEQ ID NO: 441 - protein, 9 aa, Homo sapiens
SSAPTARVF

SEQ ID NO: 442 - protein, 9 aa, Homo sapiens
SSTPHNVSW

SEQ ID NO: 443 - protein, 9 aa, Homo sapiens
STYEKALGY

SEQ ID NO: 444 - protein, 9 aa, Homo sapiens
STYPRPHEY

SEQ ID NO: 445 - protein, 9 aa, Homo sapiens
SVRPPKKSW

SEQ ID NO: 446 - protein, 9 aa, Homo sapiens
SVTGVHRLY

SEQ ID NO: 447 - protein, 9 aa, Homo sapiens
TEAEKLKSY

SEQ ID NO: 448 - protein, 9 aa, Homo sapiens
TEDEAIHKY

SEQ ID NO: 449 - protein, 9 aa, Homo sapiens
TEFDSTVTH

SEQ ID NO: 450 - protein, 9 aa, Homo sapiens
TELDQVRKW

SEQ ID NO: 451 - protein, 10 aa, Homo sapiens
TELEDARQKF

SEQ ID NO: 452 - protein, 10 aa, Homo sapiens
TENQRPKTGF

SEQ ID NO: 453 - protein, 9 aa, Homo sapiens
TEQSQIKGY

SEQ ID NO: 454 - protein, 9 aa, Homo sapiens
TEQTAPKIF

SEQ ID NO: 455 - protein, 9 aa, Homo sapiens
TESPLVKAI

SEQ ID NO: 456 - protein, 10 aa, Homo sapiens
TEVEENQKYF

SEQ ID NO: 457 - protein, 9 aa, Homo sapiens
TIIDRSFRY

SEQ ID NO: 458 - protein, 9 aa, Homo sapiens
TLWPEVQKL

SEQ ID NO: 459 - protein, 9 aa, Homo sapiens
TTHNQSRKW

SEQ ID NO: 460 - protein, 10 aa, Homo sapiens
VEISNEVRKF

SEQ ID NO: 461 - protein, 9 aa, Homo sapiens
VIAEILRGV

SEQ ID NO: 462 - protein, 9 aa, Homo sapiens
VSHSTHRTF

SEQ ID NO: 463 - protein, 9 aa, Homo sapiens
VTDETGGKY

SEQ ID NO: 464 - protein, 9 aa, Homo sapiens
VTNPHSSQW

SEQ ID NO: 465 - protein, 10 aa, Homo sapiens
VTRHPINEYY

SEQ ID NO: 466 - protein, 9 aa, Homo sapiens
YLDQTLPRA

SEQ ID NO: 467 - protein, 9 aa, Homo sapiens
YLFERIKEL

SEQ ID NO: 468 - protein, 9 aa, Homo sapiens
YLLPAIVHI

SEQ ID NO: 469 - protein, 10 aa, Homo sapiens
YLLQEPPRTV

SEQ ID NO: 470 - protein, 9 aa, Homo sapiens
YSKDRIYTY

SEQ ID NO: 471 - protein, 10 aa, Homo sapiens
ASEFPGAQHY

SEQ ID NO: 472 - protein, 9 aa, Homo sapiens
AVDAARLHY

SEQ ID NO: 473 - protein, 9 aa, Homo sapiens
AVDPRFLAY

SEQ ID NO: 474 - protein, 9 aa, Homo sapiens
DEEGKGAVY

SEQ ID NO: 475 - protein, 9 aa, Homo sapiens
DEEGLKLTF

SEQ ID NO: 476 - protein, 9 aa, Homo sapiens
DEFEFLEKA

SEQ ID NO: 477 - protein, 9 aa, Homo sapiens
DEFPLLTTK

SEQ ID NO: 478 - protein, 9 aa, Homo sapiens
DEKLNNAKY

SEQ ID NO: 479 - protein, 9 aa, Homo sapiens
DEVDMAHRF

SEQ ID NO: 480 - protein, 9 aa, Homo sapiens
DEVQVVRGH

SEQ ID NO: 481 - protein, 10 aa, Homo sapiens
DEVQVVRGHY

SEQ ID NO: 482 - protein, 9 aa, Homo sapiens
DTDHYFLRY

SEQ ID NO: 483 - protein, 10 aa, Homo sapiens
EEVEAKFKRY

SEQ ID NO: 484 - protein, 9 aa, Homo sapiens
EEYLLRQVF

SEQ ID NO: 485 - protein, 9 aa, Homo sapiens
EHFPNHQQL

SEQ ID NO: 486 - protein, 9 aa, Homo sapiens
ESDINSVRY

SEQ ID NO: 487 - protein, 11 aa, Homo sapiens
ETDSSEEISRY

SEQ ID NO: 488 - protein, 9 aa, Homo sapiens
ETELDGLRY

SEQ ID NO: 489 - protein, 9 aa, Homo sapiens
FISEFEHRV

SEQ ID NO: 490 - protein, 9 aa, Homo sapiens
FSDSKIQKY

SEQ ID NO: 491 - protein, 9 aa, Homo sapiens
FTDKSAEDY

SEQ ID NO: 492 - protein, 11 aa, Homo sapiens
GLAPPQHLIRV

SEQ ID NO: 493 - protein, 9 aa, Homo sapiens
GSDRIKDGY

SEQ ID NO: 494 - protein, 10 aa, Homo sapiens
HTELPRGDHY

SEQ ID NO: 495 - protein, 9 aa, Homo sapiens
IEFARKNVY

SEQ ID NO: 496 - protein, 10 aa, Homo sapiens
IEFEYNKSKY

SEQ ID NO: 497 - protein, 9 aa, Homo sapiens
IENPALLRW

SEQ ID NO: 498 - protein, 9 aa, Homo sapiens
ILDAGGHNV

SEQ ID NO: 499 - protein, 9 aa, Homo sapiens
ILDKKVEKV

SEQ ID NO: 500 - protein, 9 aa, Homo sapiens
ILQDRLNQV

SEQ ID NO: 501 - protein, 9 aa, Homo sapiens
ILQEREYRL

SEQ ID NO: 502 - protein, 9 aa, Homo sapiens
ILQTKLYTL

SEQ ID NO: 503 - protein, 10 aa, Homo sapiens
ISEAGKDLLY

SEQ ID NO: 504 - protein, 10 aa, Homo sapiens
ITDPDPDIRY

SEQ ID NO: 505 - protein, 9 aa, Homo sapiens
ITQGTPLKY

SEQ ID NO: 506 - protein, 9 aa, Homo sapiens
KLIDRTESL

SEQ ID NO: 507 - protein, 9 aa, Homo sapiens
KLPEKWESV

SEQ ID NO: 508 - protein, 9 aa, Homo sapiens
KLSEVNKRL

SEQ ID NO: 509 - protein, 9 aa, Homo sapiens
KMADIEYRL

SEQ ID NO: 510 - protein, 9 aa, Homo sapiens
KVIDEIYRV

SEQ ID NO: 511 - protein, 9 aa, Homo sapiens
KVQEQVHKV

SEQ ID NO: 512 - protein, 9 aa, Homo sapiens
LHKSIQNSL

SEQ ID NO: 513 - protein, 10 aa, Homo sapiens
LIDQDARDLY

SEQ ID NO: 514 - protein, 9 aa, Homo sapiens
LLDEEISRV

SEQ ID NO: 515 - protein, 9 aa, Homo sapiens
LTDADAMKY

SEQ ID NO: 516 - protein, 9 aa, Homo sapiens
LTDFIREEY

SEQ ID NO: 517 - protein, 10 aa, Homo sapiens
LTDSEKGNSY

SEQ ID NO: 518 - protein, 9 aa, Homo sapiens
MLGAGLIKI

SEQ ID NO: 519 - protein, 9 aa, Homo sapiens
MLTEKQHLL

SEQ ID NO: 520 - protein, 10 aa, Homo sapiens
NEHDYAKAAF

SEQ ID NO: 521 - protein, 9 aa, Homo sapiens
NENPLRHQF

SEQ ID NO: 522 - protein, 11 aa, Homo sapiens
NEQTLQRSWAY

SEQ ID NO: 523 - protein, 9 aa, Homo sapiens
NHNKITQSL

SEQ ID NO: 524 - protein, 9 aa, Homo sapiens
NTDRNIDKY

SEQ ID NO: 525 - protein, 9 aa, Homo sapiens
PTDPKVVVY

SEQ ID NO: 526 - protein, 9 aa, Homo sapiens
PTDTNVIRY

SEQ ID NO: 527 - protein, 9 aa, Homo sapiens
QLAEDLKKV

SEQ ID NO: 528 - protein, 9 aa, Homo sapiens
QRQQVRPVI

SEQ ID NO: 529 - protein, 10 aa, Homo sapiens
RLQEDPPAGV

SEQ ID NO: 530 - protein, 9 aa, Homo sapiens
SEFPGAQHY

SEQ ID NO: 531 - protein, 9 aa, Homo sapiens
SENELKKAY

SEQ ID NO: 532 - protein, 9 aa, Homo sapiens
SEVDLTRSF

SEQ ID NO: 533 - protein, 9 aa, Homo sapiens
SHWVKRTTL

SEQ ID NO: 534 - protein, 9 aa, Homo sapiens
TEFDSTVTH

SEQ ID NO: 535 - protein, 10 aa, Homo sapiens
THISRVGQAM

SEQ ID NO: 536 - protein, 9 aa, Homo sapiens
TLDTKPGLY

SEQ ID NO: 537 - protein, 9 aa, Homo sapiens
VETYQVHEY

SEQ ID NO: 538 - protein, 9 aa, Homo sapiens
VLDPYLLKY

SEQ ID NO: 539 - protein, 9 aa, Homo sapiens
VQDDIKESV

SEQ ID NO: 540 - protein, 9 aa, Homo sapiens
VSDFGLSRY

SEQ ID NO: 541 - protein, 9 aa, Homo sapiens
VTDETGGKY

SEQ ID NO: 542 - protein, 9 aa, Homo sapiens
VTEIDQDKY

SEQ ID NO: 543 - protein, 10 aa, Homo sapiens
WSDYSDNLKY

SEQ ID NO: 544 - protein, 9 aa, Homo sapiens
YELPWVEKY

SEQ ID NO: 545 - protein, 9 aa, Homo sapiens
YLDQTLPRA

SEQ ID NO: 546 - protein, 9 aa, Homo sapiens
YLFERIKEL

SEQ ID NO: 547 - protein, 9 aa, Homo sapiens
YLLPAIVHI

SEQ ID NO: 548 - protein, 10 aa, Homo sapiens
YMYILPYKEF

SEQ ID NO: 549 - protein, 9 aa, Homo sapiens
YSDDIPHAL

SEQ ID NO: 550 - protein, 9 aa, Homo sapiens
YSDKYGLGY

SEQ ID NO: 551 - protein, 10 aa, Homo sapiens
YSEGYPGQRY

SEQ ID NO: 552 - protein, 10 aa, Homo sapiens
YSFDNLPEKY

SEQ ID NO: 553 - protein, 10 aa, Homo sapiens
YTDFDGTRVY

SEQ ID NO: 554 - protein, 9 aa, Homo sapiens
YTFDPVTKV

SEQ ID NO: 555 - protein, 9 aa, Homo sapiens
YVEGVHQQY

SEQ ID NO: 556 - protein, 9 aa, Homo sapiens
SLSDFKFMA

SEQ ID NO: 557 - protein, 10 aa, Homo sapiens
VVHPFFLAQK

SEQ ID NO: 558 - protein, 19 aa, Artificial Sequence
  Description of Artificial Sequence Synthetic peptide
CASSLEPSGGRRWDNEQFF

SEQ ID NO: 559 - protein, 12 aa, Artificial Sequence
  Description of Artificial Sequence Synthetic peptide
CAVIVTGNQFYF

SEQ ID NO: 560 - protein, 12 aa, Artificial Sequence
  Description of Artificial Sequence Synthetic peptide
CASSEDTYGYTF

SEQ ID NO: 561 - protein, 20 aa, Artificial Sequence
  Description of Artificial Sequence Synthetic peptide
CALTGPQANAGGTSYGKLTF

SEQ ID NO: 562 - protein, 17 aa, Artificial Sequence
  Description of Artificial Sequence Synthetic peptide
CASSFQTVGSGSYEQYF

SEQ ID NO: 563 - protein, 14 aa, Artificial Sequence
  Description of Artificial Sequence Synthetic peptide
CALSGREYGNKLVF

SEQ ID NO: 564 - protein, 16 aa, Artificial Sequence
  Description of Artificial Sequence Synthetic peptide
CASSYLAGGFRDEQFF

SEQ ID NO: 565 - protein, 15 aa, Artificial Sequence
  Description of Artificial Sequence Synthetic peptide
CASSQTRTGNQPQHF

SEQ ID NO: 566 - protein, 13 aa, Artificial Sequence
  Description of Artificial Sequence Synthetic peptide
CAASTGATNKLIF

SEQ ID NO: 567 - protein, 16 aa, Artificial Sequence
  Description of Artificial Sequence Synthetic peptide
CASSLVGQGAYNEQFF

SEQ ID NO: 568 - protein, 16 aa, Artificial Sequence
  Description of Artificial Sequence Synthetic peptide
CVVNPHGSSNTGKLIF

SEQ ID NO: 569 - protein, 16 aa, Artificial Sequence
  Description of Artificial Sequence Synthetic peptide
CVVRRDSGGSNYKLTF

SEQ ID NO: 570 - protein, 15 aa, Artificial Sequence
  Description of Artificial Sequence Synthetic peptide
CASSLDPGRALGYTF

SEQ ID NO: 571 - protein, 14 aa, Artificial Sequence
  Description of Artificial Sequence Synthetic peptide
CASSFTRVAYEQYF

SEQ ID NO: 572 - protein, 17 aa, Artificial Sequence
  Description of Artificial Sequence Synthetic peptide
CASSPEPQGANFYEQYF

SEQ ID NO: 573 - protein, 15 aa, Artificial Sequence
  Description of Artificial Sequence Synthetic peptide
CASSLAAGGLSEQFF

SEQ ID NO: 574 - protein, 14 aa, Artificial Sequence
  Description of Artificial Sequence Synthetic peptide
CASSEGTVLDEQYF

SEQ ID NO: 575 - protein, 14 aa, Artificial Sequence
  Description of Artificial Sequence Synthetic peptide
CAESKLDAGNMLTF

SEQ ID NO: 576 - protein, 15 aa, Artificial Sequence
  Description of Artificial Sequence Synthetic peptide
CASSLVGQGLGEQYF

SEQ ID NO: 577 - protein, 20 aa, Artificial Sequence
  Description of Artificial Sequence Synthetic peptide
CARRRVLVAWDCSGANVLTF

SEQ ID NO: 578 - protein, 14 aa, Artificial Sequence
  Description of Artificial Sequence Synthetic peptide
CASSASDSYNEQFF

SEQ ID NO: 579 - protein, 12 aa, Artificial Sequence
  Description of Artificial Sequence Synthetic peptide
CAARDSSYKLIF

SEQ ID NO: 580 - protein, 12 aa, Artificial Sequence
  Description of Artificial Sequence Synthetic peptide
CASSLGSSTQYF

SEQ ID NO: 581 - protein, 16 aa, Artificial Sequence
  Description of Artificial Sequence Synthetic peptide
CALSEGSNFGNEKLTF

SEQ ID NO: 582 - protein, 12 aa, Artificial Sequence
  Description of Artificial Sequence Synthetic peptide
CSATGTYNEQFF

SEQ ID NO: 583 - protein, 15 aa, Artificial Sequence
  Description of Artificial Sequence Synthetic peptide
CASSFNPQGREKLFF

SEQ ID NO: 584 - protein, 13 aa, Artificial Sequence
  Description of Artificial Sequence Synthetic peptide
CVVSNNAGNMLTF

SEQ ID NO: 585 - protein, 14 aa, Artificial Sequence
  Description of Artificial Sequence Synthetic peptide
CASSPGPVQETQYF

SEQ ID NO: 586 - protein, 15 aa, Artificial Sequence
  Description of Artificial Sequence Synthetic peptide
CAVTSRGGSNYKLTF

SEQ ID NO: 587 - protein, 14 aa, Artificial Sequence
  Description of Artificial Sequence Synthetic peptide
CASSSVSSGNTIYF

SEQ ID NO: 588 - protein, 11 aa, Artificial Sequence
  Description of Artificial Sequence Synthetic peptide
CAVQGYQKVTF

SEQ ID NO: 589 - protein, 13 aa, Artificial Sequence
  Description of Artificial Sequence Synthetic peptide
CAVNIRGANNLFF

SEQ ID NO: 590 - protein, 18 aa, Artificial Sequence
  Description of Artificial Sequence Synthetic peptide
CASSSRLAGGQVSYEQYF

SEQ ID NO: 591 - protein, 15 aa, Artificial Sequence
  Description of Artificial Sequence Synthetic peptide
CASSWTSGGMNEQFF

SEQ ID NO: 592 - protein, 15 aa, Artificial Sequence
  Description of Artificial Sequence Synthetic peptide
CASSFLTDSYNEQFF

SEQ ID NO: 593 - protein, 11 aa, Artificial Sequence
  Description of Artificial Sequence Synthetic peptide
CAVRPSGRLMF

SEQ ID NO: 594 - protein, 15 aa, Artificial Sequence
  Description of Artificial Sequence Synthetic peptide
CASSQGALGNTEAFF

SEQ ID NO: 595 - protein, 14 aa, Artificial Sequence
  Description of Artificial Sequence Synthetic peptide
CQQHTDRDQETQYF

SEQ ID NO: 596 - protein, 12 aa, Artificial Sequence
  Description of Artificial Sequence Synthetic peptide
CAIPYGYNKLIF

SEQ ID NO: 597 - protein, 13 aa, Artificial Sequence
  Description of Artificial Sequence Synthetic peptide
CAWSGTGGSEAFF

SEQ ID NO: 598 - protein, 15 aa, Artificial Sequence
  Description of Artificial Sequence Synthetic peptide
CASSSRTVVNYGYTF

SEQ ID NO: 599 - protein, 15 aa, Artificial Sequence
  Description of Artificial Sequence Synthetic peptide
CSARRGGAPNTEAFF

SEQ ID NO: 600 - protein, 14 aa, Artificial Sequence
  Description of Artificial Sequence Synthetic peptide
CASSLADPTDTQYF

SEQ ID NO: 601 - protein, 16 aa, Artificial Sequence
  Description of Artificial Sequence Synthetic peptide
CLVGGLYGGSQGNLIF

SEQ ID NO: 602 - protein, 13 aa, Artificial Sequence
  Description of Artificial Sequence Synthetic peptide
CAADAGNNRKLIW

SEQ ID NO: 603 - protein, 15 aa, Artificial Sequence
  Description of Artificial Sequence Synthetic peptide
CASSYTPGPRGEQYF

SEQ ID NO: 604 - protein, 15 aa, Artificial Sequence
  Description of Artificial Sequence Synthetic peptide
CASKPTSGSVNEQFF

SEQ ID NO: 605 - protein, 14 aa, Artificial Sequence
  Description of Artificial Sequence Synthetic peptide
CAAKGGTSYGKLTF

SEQ ID NO: 606 - protein, 13 aa, Artificial Sequence
  Description of Artificial Sequence Synthetic peptide
CASSASGNQPQHF

SEQ ID NO: 607 - protein, 13 aa, Artificial Sequence
  Description of Artificial Sequence Synthetic peptide
CAESTGGYNKLIF

SEQ ID NO: 608 - protein, 13 aa, Artificial Sequence
  Description of Artificial Sequence Synthetic peptide
CSVETGVNQPQHF

SEQ ID NO: 609 - protein, 14 aa, Artificial Sequence
  Description of Artificial Sequence Synthetic peptide
CASSATSGKHEQYF

SEQ ID NO: 610 - protein, 14 aa, Artificial Sequence
  Description of Artificial Sequence Synthetic peptide
CASCLPGGNEKLFF

SEQ ID NO: 611 - protein, 15 aa, Artificial Sequence
  Description of Artificial Sequence Synthetic peptide
CAASAALNRDDKIIF

SEQ ID NO: 612 - protein, 13 aa, Artificial Sequence
  Description of Artificial Sequence Synthetic peptide
CASSPVGWREQFF

SEQ ID NO: 613 - protein, 8 aa, Artificial Sequence
  Description of Artificial Sequence Synthetic peptide
GRDDKIIF

SEQ ID NO: 614 - protein, 13 aa, Artificial Sequence
  Description of Artificial Sequence Synthetic peptide
CASSPPDSYGYTF

SEQ ID NO: 615 - protein, 13 aa, Artificial Sequence
  Description of Artificial Sequence Synthetic peptide
CAVGNTGGFKTIF

SEQ ID NO: 616 - protein, 13 aa, Artificial Sequence
  Description of Artificial Sequence Synthetic peptide
CASRVWGADTQYF

SEQ ID NO: 617 - protein, 12 aa, Artificial Sequence
  Description of Artificial Sequence Synthetic peptide
CALVTGANNLFF

SEQ ID NO: 618 - protein, 14 aa, Artificial Sequence
  Description of Artificial Sequence Synthetic peptide
CASSYDGENTEAFF

SEQ ID NO: 619 - protein, 12 aa, Artificial Sequence
  Description of Artificial Sequence Synthetic peptide
CAAVNYGQNFVF

SEQ ID NO: 620 - protein, 14 aa, Artificial Sequence
  Description of Artificial Sequence Synthetic peptide
CAASSGGTASKLTF

SEQ ID NO: 621 - protein, 17 aa, Artificial Sequence
  Description of Artificial Sequence Synthetic peptide
CALSEVSSGAGSYQLTF

SEQ ID NO: 622 - protein, 14 aa, Artificial Sequence
  Description of Artificial Sequence Synthetic peptide
CAESPSGGSYIPTF

SEQ ID NO: 623 - protein, 11 aa, Artificial Sequence
  Description of Artificial Sequence Synthetic peptide
CAVRGATKLSF

SEQ ID NO: 624 - protein, 12 aa, Artificial Sequence
  Description of Artificial Sequence Synthetic peptide
CAASTPGEKLTF

SEQ ID NO: 625 - protein, 15 aa, Artificial Sequence
  Description of Artificial Sequence Synthetic peptide
CALRGRSGGSYIPTF

SEQ ID NO: 626 - protein, 16 aa, Artificial Sequence
  Description of Artificial Sequence Synthetic peptide
CASSQSLTGGLQPQHF

SEQ ID NO: 627 - protein, 21 aa, Artificial Sequence
  Description of Artificial Sequence Synthetic peptide
CASGLVPRPSGGESNTGELFF

SEQ ID NO: 628 - protein, 13 aa, Artificial Sequence
  Description of Artificial Sequence Synthetic peptide
CASSPGNNQPQHF

SEQ ID NO: 629 - protein, 15 aa, Artificial Sequence
  Description of Artificial Sequence Synthetic peptide
CALTLFSGGYNKLIF

SEQ ID NO: 630 - protein, 12 aa, Artificial Sequence
  Description of Artificial Sequence Synthetic peptide
CSARPPGGGYTF

SEQ ID NO: 631 - protein, 15 aa, Artificial Sequence
  Description of Artificial Sequence Synthetic peptide
CALSAASGGSYIPTF

SEQ ID NO: 632 - protein, 13 aa, Artificial Sequence
  Description of Artificial Sequence Synthetic peptide
CSARMTTQETQYF

SEQ ID NO: 633 - protein, 15 aa, Artificial Sequence
  Description of Artificial Sequence Synthetic peptide
CASIGRSMGTDTQYF

SEQ ID NO: 634 - protein, 14 aa, Artificial Sequence
  Description of Artificial Sequence Synthetic peptide
CAVSEVASGSRLTF

SEQ ID NO: 635 - protein, 15 aa, Artificial Sequence
  Description of Artificial Sequence Synthetic peptide
CASSPLRVGDYGYTF

SEQ ID NO: 636 - protein, 19 aa, Artificial Sequence
  Description of Artificial Sequence Synthetic peptide
CASRSRLRPSGRSTDTQYF

SEQ ID NO: 637 - protein, 9 aa, Artificial Sequence
  Description of Artificial Sequence Synthetic peptide
PLKGNDMRF

SEQ ID NO: 638 - protein, 14 aa, Artificial Sequence
  Description of Artificial Sequence Synthetic peptide
CAPAGGTSYGKLTF

SEQ ID NO: 639 - protein, 13 aa, Artificial Sequence
  Description of Artificial Sequence Synthetic peptide
CASSGSDGLEQFF

SEQ ID NO: 640 - protein, 14 aa, Artificial Sequence
  Description of Artificial Sequence Synthetic peptide
CASTGTRSYNEQFF

SEQ ID NO: 641 - protein, 16 aa, Artificial Sequence
  Description of Artificial Sequence Synthetic peptide
CASSTGTSGRAREQYF

SEQ ID NO: 642 - protein, 11 aa, Artificial Sequence
  Description of Artificial Sequence Synthetic peptide
CSVEGSLAQYF

SEQ ID NO: 643 - protein, 15 aa, Artificial Sequence
  Description of Artificial Sequence Synthetic peptide
CSVVGTASSGNTIYF

SEQ ID NO: 644 - protein, 13 aa, Artificial Sequence
  Description of Artificial Sequence Synthetic peptide
CAVREYNFNKFYF

SEQ ID NO: 645 - protein, 14 aa, Artificial Sequence
  Description of Artificial Sequence Synthetic peptide
CASSTQGGTDTQYF

SEQ ID NO: 646 - protein, 14 aa, Artificial Sequence
  Description of Artificial Sequence Synthetic peptide
CASSPTVGAYGYTF

SEQ ID NO: 647 - protein, 13 aa, Artificial Sequence
  Description of Artificial Sequence Synthetic peptide
CAERGSNTGKLIF

SEQ ID NO: 648 - protein, 11 aa, Artificial Sequence
  Description of Artificial Sequence Synthetic peptide
CASSDGGEQFF

SEQ ID NO: 649 - protein, 11 aa, Artificial Sequence
  Description of Artificial Sequence Synthetic peptide
CASSLYSGQFF

SEQ ID NO: 650 - protein, 15 aa, Artificial Sequence
  Description of Artificial Sequence Synthetic peptide
CASSSRTSGGDEQYF

SEQ ID NO: 651 - protein, 16 aa, Artificial Sequence
  Description of Artificial Sequence Synthetic peptide
CIVIVYSGGGADGLTF

SEQ ID NO: 652 - protein, 15 aa, Artificial Sequence
  Description of Artificial Sequence Synthetic peptide
CASSRPEGVNQPQHF

SEQ ID NO: 653 - protein, 14 aa, Artificial Sequence
  Description of Artificial Sequence Synthetic peptide
CASSDGTGDYEQYF

SEQ ID NO: 654 - protein, 14 aa, Artificial Sequence
  Description of Artificial Sequence Synthetic peptide
CAVSFTGGGNKLTF

SEQ ID NO: 655 - protein, 15 aa, Artificial Sequence
  Description of Artificial Sequence Synthetic peptide
CASSAEAYSGYGYTF

SEQ ID NO: 656 - protein, 11 aa, Artificial Sequence
  Description of Artificial Sequence Synthetic peptide
CAVGGYSTLTF

SEQ ID NO: 657 - protein, 14 aa, Artificial Sequence
  Description of Artificial Sequence Synthetic peptide
CGTEGGGSNYKLTF

SEQ ID NO: 658 - protein, 14 aa, Artificial Sequence
  Description of Artificial Sequence Synthetic peptide
CASLFERGSTEAFF

SEQ ID NO: 659 - protein, 15 aa, Artificial Sequence
  Description of Artificial Sequence Synthetic peptide
CASSQQTETSYGYTF

SEQ ID NO: 660 - protein, 13 aa, Artificial Sequence
  Description of Artificial Sequence Synthetic peptide
CAVGRSSASKIIF

SEQ ID NO: 661 - protein, 15 aa, Artificial Sequence
  Description of Artificial Sequence Synthetic peptide
CASSLGGGSSYEQYF

SEQ ID NO: 662 - protein, 16 aa, Artificial Sequence
  Description of Artificial Sequence Synthetic peptide
CASSPATTMGIDTQYF

SEQ ID NO: 663 - protein, 14 aa, Artificial Sequence
  Description of Artificial Sequence Synthetic peptide
CAASGYSSASKIIF

SEQ ID NO: 664 - protein, 15 aa, Artificial Sequence
  Description of Artificial Sequence Synthetic peptide
CASSSTATPADEQFF

SEQ ID NO: 665 - protein, 13 aa, Artificial Sequence
  Description of Artificial Sequence Synthetic peptide
CASSPGNSQPQHF

SEQ ID NO: 666 - protein, 13 aa, Artificial Sequence
  Description of Artificial Sequence Synthetic peptide
CASSREGDQPQHF

SEQ ID NO: 667 - protein, 13 aa, Artificial Sequence
  Description of Artificial Sequence Synthetic peptide
CASSLVSDNEQFF

SEQ ID NO: 668 - protein, 16 aa, Artificial Sequence
  Description of Artificial Sequence Synthetic peptide
CAASRSRGSTLGRLYF

SEQ ID NO: 669 - protein, 13 aa, Artificial Sequence
  Description of Artificial Sequence Synthetic peptide
CAVIIQGAQKLVF

SEQ ID NO: 670 - protein, 13 aa, Artificial Sequence
  Description of Artificial Sequence Synthetic peptide
CAVEATGTYKYIF

SEQ ID NO: 671 - protein, 11 aa, Artificial Sequence
  Description of Artificial Sequence Synthetic peptide
CASSDGQIAFF

SEQ ID NO: 672 - protein, 16 aa, Artificial Sequence
  Description of Artificial Sequence Synthetic peptide
CASTTGSGRINNEQFF

SEQ ID NO: 673 - protein, 16 aa, Artificial Sequence
  Description of Artificial Sequence Synthetic peptide
CASSRPGTSGTDTQYF

SEQ ID NO: 674 - protein, 18 aa, Artificial Sequence
  Description of Artificial Sequence Synthetic peptide
CASSLPGTSGRYSYEQYF

SEQ ID NO: 675 - protein, 15 aa, Artificial Sequence
  Description of Artificial Sequence Synthetic peptide
CIVRPYGGSQGNLIF

SEQ ID NO: 676 - protein, 12 aa, Artificial Sequence
  Description of Artificial Sequence Synthetic peptide
CASISSNGEAFF

SEQ ID NO: 677 - protein, 14 aa, Artificial Sequence
  Description of Artificial Sequence Synthetic peptide
CASSLLAVTDTQYF

SEQ ID NO: 678 - protein, 15 aa, Artificial Sequence
  Description of Artificial Sequence Synthetic peptide
CALWEGQGGSEKLVF

SEQ ID NO: 679 - protein, 18 aa, Artificial Sequence
  Description of Artificial Sequence Synthetic peptide
CASSSGTSGGRHTYEQYF

SEQ ID NO: 680 - protein, 13 aa, Artificial Sequence
  Description of Artificial Sequence Synthetic peptide
CATELRYTGELFF

SEQ ID NO: 681 - protein, 14 aa, Artificial Sequence
  Description of Artificial Sequence Synthetic peptide
CASSSGTGNQPQHF

SEQ ID NO: 682 - protein, 12 aa, Artificial Sequence
  Description of Artificial Sequence Synthetic peptide
CASSETPRDSFF

SEQ ID NO: 683 - protein, 11 aa, Artificial Sequence
  Description of Artificial Sequence Synthetic peptide
CAVNTGNQFYF

SEQ ID NO: 684 - protein, 15 aa, Artificial Sequence
  Description of Artificial Sequence Synthetic peptide
CAASIAGGGADGLTF

SEQ ID NO: 685 - protein, 13 aa, Artificial Sequence
  Description of Artificial Sequence Synthetic peptide
CAECNNAGNMLTF

SEQ ID NO: 686 - protein, 10 aa, Artificial Sequence
  Description of Artificial Sequence Synthetic peptide
CASSELIPTF

SEQ ID NO: 687 - protein, 13 aa, Artificial Sequence
  Description of Artificial Sequence Synthetic peptide
CASSPGGGQPQHF

SEQ ID NO: 688 - protein, 15 aa, Artificial Sequence
  Description of Artificial Sequence Synthetic peptide
CASSPQGGAGNTIYF

SEQ ID NO: 689 - protein, 13 aa, Artificial Sequence
  Description of Artificial Sequence Synthetic peptide
CASSARDRAVQYF

SEQ ID NO: 690 - protein, 14 aa, Artificial Sequence
  Description of Artificial Sequence Synthetic peptide
CAVGADTGRRALTF

SEQ ID NO: 691 - protein, 13 aa, Artificial Sequence
  Description of Artificial Sequence Synthetic peptide
CSVYIATNEKLFF

SEQ ID NO: 692 - protein, 13 aa, Artificial Sequence
  Description of Artificial Sequence Synthetic peptide
CALNDNAGNMLTF

SEQ ID NO: 693 - protein, 18 aa, Artificial Sequence
  Description of Artificial Sequence Synthetic peptide
CASSQRGGGLRGTDTQYF

SEQ ID NO: 694 - protein, 18 aa, Artificial Sequence
  Description of Artificial Sequence Synthetic peptide
CATSDFRGGGLAKNIQYF

SEQ ID NO: 695 - protein, 14 aa, Artificial Sequence
  Description of Artificial Sequence Synthetic peptide
CASSPLAGVYEQFF

SEQ ID NO: 696 - protein, 16 aa, Artificial Sequence
  Description of Artificial Sequence Synthetic peptide
CASSQVFGTGASPLHF

SEQ ID NO: 697 - protein, 16 aa, Artificial Sequence
  Description of Artificial Sequence Synthetic peptide
CAMRLNYGGSQGNLIF

SEQ ID NO: 698 - protein, 13 aa, Artificial Sequence
  Description of Artificial Sequence Synthetic peptide
CASSINSNQPQHF

SEQ ID NO: 699 - protein, 13 aa, Artificial Sequence
  Description of Artificial Sequence Synthetic peptide
CAMSSNQGGKLIF

SEQ ID NO: 700 - protein, 13 aa, Artificial Sequence
  Description of Artificial Sequence Synthetic peptide
CASSQGARTSYTF

SEQ ID NO: 701 - protein, 14 aa, Artificial Sequence
  Description of Artificial Sequence Synthetic peptide
CAVISNFGNEKLTF

SEQ ID NO: 702 - protein, 15 aa, Artificial Sequence
  Description of Artificial Sequence Synthetic peptide
CASSLADRGDEKLFF

SEQ ID NO: 703 - protein, 13 aa, Artificial Sequence
  Description of Artificial Sequence Synthetic peptide
CAAPRGGAQKLVF

SEQ ID NO: 704 - protein, 17 aa, Artificial Sequence
  Description of Artificial Sequence Synthetic peptide
CASSLMRLAGGRDEQFF

SEQ ID NO: 705 - protein, 16 aa, Artificial Sequence
  Description of Artificial Sequence Synthetic peptide
CASRDSSGSSYNEQFF

SEQ ID NO: 706 - protein, 14 aa, Artificial Sequence
  Description of Artificial Sequence Synthetic peptide
CATSRQGSNQPQHF

SEQ ID NO: 707 - protein, 12 aa, Artificial Sequence
  Description of Artificial Sequence Synthetic peptide
CVVSAFNNDMRF

SEQ ID NO: 708 - protein, 16 aa, Artificial Sequence
  Description of Artificial Sequence Synthetic peptide
CASSPGQRYSNQPQHF

SEQ ID NO: 709 - protein, 14 aa, Artificial Sequence
  Description of Artificial Sequence Synthetic peptide
CAGLMKTSYDKVIF

SEQ ID NO: 710 - protein, 11 aa, Artificial Sequence
  Description of Artificial Sequence Synthetic peptide
CASSPGHTIYF

SEQ ID NO: 711 - protein, 11 aa, Artificial Sequence
  Description of Artificial Sequence Synthetic peptide
CASSTARAQYF

SEQ ID NO: 712 - protein, 14 aa, Artificial Sequence
  Description of Artificial Sequence Synthetic peptide
CAVNSGGGADGLTF

SEQ ID NO: 713 - protein, 14 aa, Artificial Sequence
  Description of Artificial Sequence Synthetic peptide
CASSPGQGRQPQHF

SEQ ID NO: 714 - protein, 13 aa, Artificial Sequence
  Description of Artificial Sequence Synthetic peptide
CAVFQGGSEKLVF

SEQ ID NO: 715 - protein, 14 aa, Artificial Sequence
  Description of Artificial Sequence Synthetic peptide
CAAADFSGTYKYIF

SEQ ID NO: 716 - protein, 11 aa, Artificial Sequence
  Description of Artificial Sequence Synthetic peptide
CSARAYNEQFF

SEQ ID NO: 717 - protein, 13 aa, Artificial Sequence
  Description of Artificial Sequence Synthetic peptide
CAVNGQGNNRLAF

SEQ ID NO: 718 - protein, 14 aa, Artificial Sequence
  Description of Artificial Sequence Synthetic peptide
CATSGGASNQPQHF

SEQ ID NO: 719 - protein, 15 aa, Artificial Sequence
  Description of Artificial Sequence Synthetic peptide
CAVGAGTGTASKLTF

SEQ ID NO: 720 - protein, 13 aa, Artificial Sequence
  Description of Artificial Sequence Synthetic peptide
CASNTRESYEQYF

SEQ ID NO: 721 - protein, 14 aa, Artificial Sequence
  Description of Artificial Sequence Synthetic peptide
CASSQDNRVHEQYF

SEQ ID NO: 722 - protein, 13 aa, Artificial Sequence
  Description of Artificial Sequence Synthetic peptide
CASSFRGYEKLFF

SEQ ID NO: 723 - protein, 14 aa, Artificial Sequence
  Description of Artificial Sequence Synthetic peptide
CASTPTGLPYGYTF

SEQ ID NO: 724 - protein, 14 aa, Artificial Sequence
  Description of Artificial Sequence Synthetic peptide
CAPVSSGSARQLTF

SEQ ID NO: 725 - protein, 14 aa, Artificial Sequence
  Description of Artificial Sequence Synthetic peptide
CASSLVRTADTQYF

SEQ ID NO: 726 - protein, 15 aa, Artificial Sequence
  Description of Artificial Sequence Synthetic peptide
CASTNLAGGYNEQFF

SEQ ID NO: 727 - protein, 13 aa, Artificial Sequence
  Description of Artificial Sequence Synthetic peptide
CAVEGGSQGNLIF

SEQ ID NO: 728 - protein, 16 aa, Artificial Sequence
  Description of Artificial Sequence Synthetic peptide
CASSLSASGVTDTQYF

SEQ ID NO: 729 - protein, 15 aa, Artificial Sequence
  Description of Artificial Sequence Synthetic peptide
CASSIAGSGGDEQFF

SEQ ID NO: 730 - protein, 13 aa, Artificial Sequence
  Description of Artificial Sequence Synthetic peptide
CASTFGRGNIQYF

SEQ ID NO: 731 - protein, 14 aa, Artificial Sequence
  Description of Artificial Sequence Synthetic peptide
CAVSDLGDGQKLLF

SEQ ID NO: 732 - protein, 11 aa, Artificial Sequence
  Description of Artificial Sequence Synthetic peptide
CAAGGYQKVTF

SEQ ID NO: 733 - protein, 13 aa, Artificial Sequence
  Description of Artificial Sequence Synthetic peptide
CATSSNGYNEQFF

SEQ ID NO: 734 - protein, 15 aa, Artificial Sequence
  Description of Artificial Sequence Synthetic peptide
CASSLVSFSPETQYF

SEQ ID NO: 735 - protein, 14 aa, Artificial Sequence
  Description of Artificial Sequence Synthetic peptide
CASSSARGVPEAFF

SEQ ID NO: 736 - protein, 14 aa, Artificial Sequence
  Description of Artificial Sequence Synthetic peptide
CASSSREALYEQYF

SEQ ID NO: 737 - protein, 12 aa, Artificial Sequence
  Description of Artificial Sequence Synthetic peptide
CASSWTPSEQFF

SEQ ID NO: 738 - protein, 12 aa, Artificial Sequence
  Description of Artificial Sequence Synthetic peptide
CASSLGPPPLHF

SEQ ID NO: 739 - protein, 14 aa, Artificial Sequence
  Description of Artificial Sequence Synthetic peptide
CALDMRNYGQNFVF

SEQ ID NO: 740 - protein, 17 aa, Artificial Sequence
  Description of Artificial Sequence Synthetic peptide
CAENTRAGGTSYGKLTF

SEQ ID NO: 741 - protein, 17 aa, Artificial Sequence
  Description of Artificial Sequence Synthetic peptide
CAYRSARSGAGSYQLTF

SEQ ID NO: 742 - protein, 13 aa, Artificial Sequence
  Description of Artificial Sequence Synthetic peptide
CAVGTSGTYKYIF

SEQ ID NO: 743 - protein, 14 aa, Artificial Sequence
  Description of Artificial Sequence Synthetic peptide
CASTGQSSYNEQFF

SEQ ID NO: 744 - protein, 15 aa, Artificial Sequence
  Description of Artificial Sequence Synthetic peptide
CASSASHSSYNEQFF

SEQ ID NO: 745 - protein, 13 aa, Artificial Sequence
  Description of Artificial Sequence Synthetic peptide
CATANNAGNMLTF

SEQ ID NO: 746 - protein, 16 aa, Artificial Sequence
  Description of Artificial Sequence Synthetic peptide
CAAGGNYGGSQGNLIF

SEQ ID NO: 747 - protein, 15 aa, Artificial Sequence
  Description of Artificial Sequence Synthetic peptide
CATDYRTGGSYIPTF

SEQ ID NO: 748 - protein, 18 aa, Artificial Sequence
  Description of Artificial Sequence Synthetic peptide
CAVRDPSREPGRKAYLRT

SEQ ID NO: 749 - protein, 12 aa, Artificial Sequence
  Description of Artificial Sequence Synthetic peptide
CAFDSGYSTLTF

SEQ ID NO: 750 - protein, 12 aa, Artificial Sequence
  Description of Artificial Sequence Synthetic peptide
CAVDRDQGNLIF

SEQ ID NO: 751 - protein, 12 aa, Artificial Sequence
  Description of Artificial Sequence Synthetic peptide
CAVRGTGNQFYF

SEQ ID NO: 752 - protein, 14 aa, Artificial Sequence
  Description of Artificial Sequence Synthetic peptide
CAVRDGASNYQLIW

SEQ ID NO: 753 - protein, 16 aa, Artificial Sequence
  Description of Artificial Sequence Synthetic peptide
CALSENILTGANNLFF

SEQ ID NO: 754 - protein, 13 aa, Artificial Sequence
  Description of Artificial Sequence Synthetic peptide
CALSERAGNQFYF

SEQ ID NO: 755 - protein, 12 aa, Artificial Sequence
  Description of Artificial Sequence Synthetic peptide
CAGAYYGQNFVF

SEQ ID NO: 756 - protein, 12 aa, Artificial Sequence
  Description of Artificial Sequence Synthetic peptide
CAVTNRDDKIIF

SEQ ID NO: 757 - protein, 11 aa, Artificial Sequence
  Description of Artificial Sequence Synthetic peptide
CAVRYGQNFVF

SEQ ID NO: 758 - protein, 13 aa, Artificial Sequence
  Description of Artificial Sequence Synthetic peptide
CLVGWDQGGKLIF

SEQ ID NO: 759 - protein, 14 aa, Artificial Sequence
  Description of Artificial Sequence Synthetic peptide
CAVRDQRRDDKIIF

SEQ ID NO: 760 - protein, 13 aa, Artificial Sequence
  Description of Artificial Sequence Synthetic peptide
CAVNAGNNRKLIW

SEQ ID NO: 761 - protein, 14 aa, Artificial Sequence
  Description of Artificial Sequence Synthetic peptide
CVVDSGAGSYQLTF

SEQ ID NO: 762 - protein, 14 aa, Artificial Sequence
  Description of Artificial Sequence Synthetic peptide
CAVDLREGRRALTF

SEQ ID NO: 763 - protein, 13 aa, Artificial Sequence
  Description of Artificial Sequence Synthetic peptide
CVVSKGSARQLTF

SEQ ID NO: 764 - protein, 13 aa, Artificial Sequence
  Description of Artificial Sequence Synthetic peptide
CAMGGSTLGRLYF

SEQ ID NO: 765 - protein, 12 aa, Artificial Sequence
  Description of Artificial Sequence Synthetic peptide
CAASMAGTALIF

SEQ ID NO: 766 - protein, 13 aa, Artificial Sequence
  Description of Artificial Sequence Synthetic peptide
CAPNAGNNRKLIW

SEQ ID NO: 767 - protein, 13 aa, Artificial Sequence
  Description of Artificial Sequence Synthetic peptide
CALKVGSQGNLIF

SEQ ID NO: 768 - protein, 10 aa, Artificial Sequence
  Description of Artificial Sequence Synthetic peptide
CATNDYKLSF

SEQ ID NO: 769 - protein, 11 aa, Artificial Sequence
  Description of Artificial Sequence Synthetic peptide
CATDNDYKLSF

SEQ ID NO: 770 - protein, 16 aa, Artificial Sequence
  Description of Artificial Sequence Synthetic peptide
CALSEAGSSNTGKLIF

SEQ ID NO: 771 - protein, 19 aa, Artificial Sequence
  Description of Artificial Sequence Synthetic peptide
CALSAYFNAGGTSYGKLTF

SEQ ID NO: 772 - protein, 14 aa, Artificial Sequence
  Description of Artificial Sequence Synthetic peptide
CALGVNTGFQKLVF

SEQ ID NO: 773 - protein, 15 aa, Artificial Sequence
  Description of Artificial Sequence Synthetic peptide
CALSDTSGGYNKLIF

SEQ ID NO: 774 - protein, 11 aa, Artificial Sequence
  Description of Artificial Sequence Synthetic peptide
CAVHGTDKLIF

SEQ ID NO: 775 - protein, 13 aa, Artificial Sequence
  Description of Artificial Sequence Synthetic peptide
CAVTIQGAQKLVF

SEQ ID NO: 776 - protein, 8 aa, Artificial Sequence
  Description of Artificial Sequence Synthetic peptide
CAGEFYYF

SEQ ID NO: 777 - protein, 16 aa, Artificial Sequence
  Description of Artificial Sequence Synthetic peptide
CAGDRGTDNYGQNFVF

SEQ ID NO: 778 - protein, 15 aa, Artificial Sequence
  Description of Artificial Sequence Synthetic peptide
CAYRSAETSGSRLTF

SEQ ID NO: 779 - protein, 14 aa, Artificial Sequence
  Description of Artificial Sequence Synthetic peptide
CAVDIGTSYGKLTF

SEQ ID NO: 780 - protein, 12 aa, Artificial Sequence
  Description of Artificial Sequence Synthetic peptide
CLVGGGDYKLSF

SEQ ID NO: 781 - protein, 16 aa, Artificial Sequence
  Description of Artificial Sequence Synthetic peptide
CVVSLFYGGATNKLIF

SEQ ID NO: 782 - protein, 15 aa, Artificial Sequence
  Description of Artificial Sequence Synthetic peptide
CAVEGPGGSQGNLIF

SEQ ID NO: 783 - protein, 12 aa, Artificial Sequence
  Description of Artificial Sequence Synthetic peptide
CAAIQGAQKLVF

SEQ ID NO: 784 - protein, 12 aa, Artificial Sequence
  Description of Artificial Sequence Synthetic peptide
CAEESNDYKLSF

SEQ ID NO: 785 - protein, 10 aa, Artificial Sequence
  Description of Artificial Sequence Synthetic peptide
CAESDYQLIW

SEQ ID NO: 786 - protein, 10 aa, Homo sapiens
ISWMKGVPGK

SEQ ID NO: 787 - protein, 12 aa, Artificial Sequence
  Description of Artificial Sequence Synthetic peptide
CASSQRGHGYTF

SEQ ID NO: 788 - protein, 14 aa, Artificial Sequence
  Description of Artificial Sequence Synthetic peptide
CAVRDISDDYKLSF

SEQ ID NO: 789 - protein, 13 aa, Artificial Sequence
  Description of Artificial Sequence Synthetic peptide
CAVTSYGNNRLAF

SEQ ID NO: 790 - protein, 11 aa, Artificial Sequence
  Description of Artificial Sequence Synthetic peptide
CAVGDDYKLSF

SEQ ID NO: 791 - protein, 14 aa, Artificial Sequence
  Description of Artificial Sequence Synthetic peptide
CAVDSGGSNYKLTF

SEQ ID NO: 792 - protein, 12 aa, Artificial Sequence
  Description of Artificial Sequence Synthetic peptide
CLQGGSARQLTF

SEQ ID NO: 793 - protein, 13 aa, Artificial Sequence
  Description of Artificial Sequence Synthetic peptide
CAYRSLTGNQFYF

SEQ ID NO: 794 - protein, 14 aa, Artificial Sequence
  Description of Artificial Sequence Synthetic peptide
CAVTLNNAGNMLTF

SEQ ID NO: 795 - protein, 12 aa, Artificial Sequence
  Description of Artificial Sequence Synthetic peptide
CAVNTGFQKLVF

SEQ ID NO: 796 - protein, 12 aa, Artificial Sequence
  Description of Artificial Sequence Synthetic peptide
CSVVSPDYGYTF

SEQ ID NO: 797 - protein, 13 aa, Artificial Sequence
  Description of Artificial Sequence Synthetic peptide
CASSDQGIDIQYF

SEQ ID NO: 798 - protein, 15 aa, Artificial Sequence
  Description of Artificial Sequence Synthetic peptide
CASSPEAGGANGYTF

SEQ ID NO: 799 - protein, 15 aa, Artificial Sequence
  Description of Artificial Sequence Synthetic peptide
CASSLPGAPTDTQYF

SEQ ID NO: 800 - protein, 14 aa, Artificial Sequence
  Description of Artificial Sequence Synthetic peptide
CASTIWRGYTEAFF

SEQ ID NO: 801 - protein, 14 aa, Artificial Sequence
  Description of Artificial Sequence Synthetic peptide
CASSFERGGETQYF

SEQ ID NO: 802 - protein, 11 aa, Artificial Sequence
  Description of Artificial Sequence Synthetic peptide
CASSFIQPQHF

SEQ ID NO: 803 - protein, 14 aa, Artificial Sequence
  Description of Artificial Sequence Synthetic peptide
CAVSEGGGYQKVTF

SEQ ID NO: 804 - protein, 17 aa, Artificial Sequence
  Description of Artificial Sequence Synthetic peptide
CAVSDGAGGTSYGKLTF

SEQ ID NO: 805 - protein, 13 aa, Artificial Sequence
  Description of Artificial Sequence Synthetic peptide
CAVRDRGGGKLIF

SEQ ID NO: 806 - protein, 9 aa, Artificial Sequence
  Description of Artificial Sequence Synthetic peptide
CAVNYQLIW

SEQ ID NO: 807 - protein, 14 aa, Artificial Sequence
  Description of Artificial Sequence Synthetic peptide
CAVRDERRGNVLHC

SEQ ID NO: 808 - protein, 12 aa, Artificial Sequence
  Description of Artificial Sequence Synthetic peptide
CAVIAAGNKLTF

SEQ ID NO: 809 - protein, 14 aa, Artificial Sequence
  Description of Artificial Sequence Synthetic peptide
CAVKGPQAGTALIF

SEQ ID NO: 810 - protein, 15 aa, Artificial Sequence
  Description of Artificial Sequence Synthetic peptide
CASSETVPSLDEQYF

SEQ ID NO: 811 - protein, 15 aa, Artificial Sequence
  Description of Artificial Sequence Synthetic peptide
CASSLTPLAWNEQFF

SEQ ID NO: 812 - protein, 14 aa, Artificial Sequence
  Description of Artificial Sequence Synthetic peptide
CASSPGQGSYGYTF

SEQ ID NO: 813 - protein, 16 aa, Artificial Sequence
  Description of Artificial Sequence Synthetic peptide
CASSVHPDRGLGEQYF

SEQ ID NO: 814 - protein, 14 aa, Artificial Sequence
  Description of Artificial Sequence Synthetic peptide
CASSREGSTDTQYF

SEQ ID NO: 815 - protein, 15 aa, Artificial Sequence
  Description of Artificial Sequence Synthetic peptide
CASSHRGSSYNEQFF

SEQ ID NO: 816 - protein, 10 aa, Artificial Sequence
  Description of Artificial Sequence Synthetic peptide
CASSHGGYTF

SEQ ID NO: 817 - protein, 14 aa, Artificial Sequence
  Description of Artificial Sequence Synthetic peptide
CASSLVSRGDEQFF

SEQ ID NO: 818 - protein, 14 aa, Artificial Sequence
  Description of Artificial Sequence Synthetic peptide
CASSFRSAGGEQFF

SEQ ID NO: 819 - protein, 14 aa, Artificial Sequence
  Description of Artificial Sequence Synthetic peptide
CAVGGYSSASKIIF

SEQ ID NO: 820 - protein, 14 aa, Artificial Sequence
  Description of Artificial Sequence Synthetic peptide
CAVSFTHPDYKLSF

SEQ ID NO: 821 - protein, 15 aa, Artificial Sequence
  Description of Artificial Sequence Synthetic peptide
CVVSDIRAGTYKYIF

SEQ ID NO: 822 - protein, 14 aa, Artificial Sequence
  Description of Artificial Sequence Synthetic peptide
CASSPGQVWNGYTF

SEQ ID NO: 823 - protein, 15 aa, Artificial Sequence
  Description of Artificial Sequence Synthetic peptide
CASSRTGGYSGPQHF

SEQ ID NO: 824 - protein, 12 aa, Artificial Sequence
  Description of Artificial Sequence Synthetic peptide
CASSLGADTQYF

SEQ ID NO: 825 - protein, 15 aa, Artificial Sequence
  Description of Artificial Sequence Synthetic peptide
CASTSGFSGANVLTF

SEQ ID NO: 826 - protein, 13 aa, Artificial Sequence
  Description of Artificial Sequence Synthetic peptide
CASSQVTGYEQYF

SEQ ID NO: 827 - protein, 11 aa, Artificial Sequence
  Description of Artificial Sequence Synthetic peptide
CASSQNYEQYF

SEQ ID NO: 828 - protein, 13 aa, Artificial Sequence
  Description of Artificial Sequence Synthetic peptide
CASSGGITDSQFF

SEQ ID NO: 829 - protein, 15 aa, Artificial Sequence
  Description of Artificial Sequence Synthetic peptide
CAVSPPGGFGNVLHC

SEQ ID NO: 830 - protein, 14 aa, Artificial Sequence
  Description of Artificial Sequence Synthetic peptide
CAVSAETGANNLFF

SEQ ID NO: 831 - protein, 15 aa, Artificial Sequence
  Description of Artificial Sequence Synthetic peptide
CAVSSFSGGYNKLIF

SEQ ID NO: 832 - protein, 15 aa, Artificial Sequence
  Description of Artificial Sequence Synthetic peptide
CAVIPFSGGYNKLIF

SEQ ID NO: 833 - protein, 20 aa, Artificial Sequence
  Description of Artificial Sequence Synthetic peptide
CATSDSKQGGTHSSYNEQFF

SEQ ID NO: 834 - protein, 14 aa, Artificial Sequence
  Description of Artificial Sequence Synthetic peptide
CASSQELGSYEQYF

SEQ ID NO: 835 - protein, 17 aa, Artificial Sequence
  Description of Artificial Sequence Synthetic peptide
CASSPSGGSHSTDTQYF

SEQ ID NO: 836 - protein, 17 aa, Artificial Sequence
  Description of Artificial Sequence Synthetic peptide
CASSPSGGPHSTDTQYF

SEQ ID NO: 837 - protein, 12 aa, Artificial Sequence
  Description of Artificial Sequence Synthetic peptide
CASSQGEGGYTF

SEQ ID NO: 838 - protein, 13 aa, Artificial Sequence
  Description of Artificial Sequence Synthetic peptide
CAVTNFGNEKLTF

SEQ ID NO: 839 - protein, 18 aa, Artificial Sequence
  Description of Artificial Sequence Synthetic peptide
CVVSEKGRDNTNAGKSTF

SEQ ID NO: 840 - protein, 7 aa, Artificial Sequence
  Description of Artificial Sequence Synthetic peptide
CVVRASI

SEQ ID NO: 841 - protein, 12 aa, Artificial Sequence
  Description of Artificial Sequence Synthetic peptide
CVFPTGANNLFF

SEQ ID NO: 842 - protein, 13 aa, Artificial Sequence
  Description of Artificial Sequence Synthetic peptide
CAVSSGGYQKVTF

SEQ ID NO: 843 - protein, 12 aa, Artificial Sequence
  Description of Artificial Sequence Synthetic peptide
CVLDNYGQNFVF

SEQ ID NO: 844 - protein, 12 aa, Artificial Sequence
  Description of Artificial Sequence Synthetic peptide
CVVSAFTDKLIF

SEQ ID NO: 845 - protein, 14 aa, Artificial Sequence
  Description of Artificial Sequence Synthetic peptide
CASSQERRGNEQFF

SEQ ID NO: 846 - protein, 17 aa, Artificial Sequence
  Description of Artificial Sequence Synthetic peptide
CASSFQSTNPSTDTQYF

SEQ ID NO: 847 - protein, 13 aa, Artificial Sequence
  Description of Artificial Sequence Synthetic peptide
CATAGGQDEKLFF

SEQ ID NO: 848 - protein, 15 aa, Artificial Sequence
  Description of Artificial Sequence Synthetic peptide
CASSFRDRGSPEQFF

SEQ ID NO: 849 - protein, 14 aa, Artificial Sequence
  Description of Artificial Sequence Synthetic peptide
CASSQAGGRDEQYF

SEQ ID NO: 850 - protein, 16 aa, Artificial Sequence
  Description of Artificial Sequence Synthetic peptide
CASSLGGRHYNSPLHF

SEQ ID NO: 851 - protein, 16 aa, Artificial Sequence
  Description of Artificial Sequence Synthetic peptide
CSARDPSLGRGYEQYF

SEQ ID NO: 852 - protein, 13 aa, Artificial Sequence
  Description of Artificial Sequence Synthetic peptide
CAVRSTNAGKSTF

SEQ ID NO: 853 - protein, 13 aa, Artificial Sequence
  Description of Artificial Sequence Synthetic peptide
CAYRDYNTDKLIF

SEQ ID NO: 854 - protein, 12 aa, Artificial Sequence
  Description of Artificial Sequence Synthetic peptide
CAVEAGRRALTF

SEQ ID NO: 855 - protein, 15 aa, Artificial Sequence
  Description of Artificial Sequence Synthetic peptide
CAVSETMDSSYKLIF

SEQ ID NO: 856 - protein, 15 aa, Artificial Sequence
  Description of Artificial Sequence Synthetic peptide
CAVSVSGGSNYKLTF

SEQ ID NO: 857 - protein, 13 aa, Artificial Sequence
  Description of Artificial Sequence Synthetic peptide
CAFISNAGNMLTF

SEQ ID NO: 858 - protein, 14 aa, Artificial Sequence
  Description of Artificial Sequence Synthetic peptide
CAVRPGNYGQNFVF

SEQ ID NO: 859 - protein, 16 aa, Artificial Sequence
  Description of Artificial Sequence Synthetic peptide
CASMLNYGGATNKLIF

SEQ ID NO: 860 - protein, 11 aa, Artificial Sequence
  Description of Artificial Sequence Synthetic peptide
PGSRGRRALTF

SEQ ID NO: 861 - protein, 12 aa, Artificial Sequence
  Description of Artificial Sequence Synthetic peptide
CASSRQRETQYF

SEQ ID NO: 862 - protein, 13 aa, Artificial Sequence
  Description of Artificial Sequence Synthetic peptide
CATSDFDGGGYTF

SEQ ID NO: 863 - protein, 12 aa, Artificial Sequence
  Description of Artificial Sequence Synthetic peptide
CASSDMNTEAFF

SEQ ID NO: 864 - protein, 13 aa, Artificial Sequence
  Description of Artificial Sequence Synthetic peptide
CASSLGAAGELFF

SEQ ID NO: 865 - protein, 14 aa, Artificial Sequence
  Description of Artificial Sequence Synthetic peptide
CASRLGTGGTEAFF

SEQ ID NO: 866 - protein, 14 aa, Artificial Sequence
  Description of Artificial Sequence Synthetic peptide
CASSFRGLSYGYTF

SEQ ID NO: 867 - protein, 15 aa, Artificial Sequence
  Description of Artificial Sequence Synthetic peptide
CASSSRTGAYNEQFF

SEQ ID NO: 868 - protein, 16 aa, Artificial Sequence
  Description of Artificial Sequence Synthetic peptide
CATSDWGGVNTGELFF

SEQ ID NO: 869 - protein, 17 aa, Artificial Sequence
  Description of Artificial Sequence Synthetic peptide
CASSPRQERSYNSPLHF

SEQ ID NO: 870 - protein, 14 aa, Artificial Sequence
  Description of Artificial Sequence Synthetic peptide
CASSYAGTSGEQFF

SEQ ID NO: 871 - protein, 13 aa, Artificial Sequence
  Description of Artificial Sequence Synthetic peptide
CASSAGQGYGYTF

SEQ ID NO: 872 - protein, 12 aa, Artificial Sequence
  Description of Artificial Sequence Synthetic peptide
CTSSQAANEQFF

SEQ ID NO: 873 - protein, 12 aa, Artificial Sequence
  Description of Artificial Sequence Synthetic peptide
CSARDGTYEQYF

SEQ ID NO: 874 - protein, 19 aa, Artificial Sequence
  Description of Artificial Sequence Synthetic peptide
CASTGSFGPQTGYTDTQYF

SEQ ID NO: 875 - protein, 15 aa, Artificial Sequence
  Description of Artificial Sequence Synthetic peptide
CVVSPSDGGYQKVTF

SEQ ID NO: 876 - protein, 12 aa, Artificial Sequence
  Description of Artificial Sequence Synthetic peptide
CAVRRAGNMLTF

SEQ ID NO: 877 - protein, 13 aa, Artificial Sequence
  Description of Artificial Sequence Synthetic peptide
CAVGNTGFQKLVF

SEQ ID NO: 878 - protein, 16 aa, Artificial Sequence
  Description of Artificial Sequence Synthetic peptide
CAVNKEIKAAGNKLTF

SEQ ID NO: 879 - protein, 15 aa, Artificial Sequence
  Description of Artificial Sequence Synthetic peptide
CARDHSGAGSYQLTF

SEQ ID NO: 880 - protein, 16 aa, Artificial Sequence
  Description of Artificial Sequence Synthetic peptide
CAVAASTTDSWGKLQF

SEQ ID NO: 881 - protein, 12 aa, Artificial Sequence
  Description of Artificial Sequence Synthetic peptide
CAVTDSTDKLIF

SEQ ID NO: 882 - protein, 12 aa, Artificial Sequence
  Description of Artificial Sequence Synthetic peptide
CAVNNAGNMLTF

SEQ ID NO: 883 - protein, 10 aa, Artificial Sequence
  Description of Artificial Sequence Synthetic peptide
CAASAQQEPF

SEQ ID NO: 884 - protein, 15 aa, Artificial Sequence
  Description of Artificial Sequence Synthetic peptide
CAVNSVDNAGNMLTF

SEQ ID NO: 885 - protein, 9 aa, Artificial Sequence
  Description of Artificial Sequence Synthetic peptide
CARNNDMRF

SEQ ID NO: 886 - protein, 15 aa, Artificial Sequence
  Description of Artificial Sequence Synthetic peptide
CAVNPEGYNNNDMRF

SEQ ID NO: 887 - protein, 18 aa, Artificial Sequence
  Description of Artificial Sequence Synthetic peptide
CAVTSGRISNFGNEKLTF

SEQ ID NO: 888 - protein, 11 aa, Artificial Sequence
  Description of Artificial Sequence Synthetic peptide
CAMGYGQNFVF

SEQ ID NO: 889 - protein, 12 aa, Artificial Sequence
  Description of Artificial Sequence Synthetic peptide
CAAHTGGFKTIF

SEQ ID NO: 890 - protein, 13 aa, Artificial Sequence
  Description of Artificial Sequence Synthetic peptide
CAMSGDSNYQLIW

SEQ ID NO: 891 - protein, 13 aa, Artificial Sequence
  Description of Artificial Sequence Synthetic peptide
CAMNLGNNNDMRF

SEQ ID NO: 892 - protein, 14 aa, Artificial Sequence
  Description of Artificial Sequence Synthetic peptide
CASLRTGEGNEQFF

SEQ ID NO: 893 - protein, 15 aa, Artificial Sequence
  Description of Artificial Sequence Synthetic peptide
CASSPTGEESDTQYF

SEQ ID NO: 894 - protein, 15 aa, Artificial Sequence
  Description of Artificial Sequence Synthetic peptide
CASSHMRGGRDEQFF

SEQ ID NO: 895 - protein, 14 aa, Artificial Sequence
  Description of Artificial Sequence Synthetic peptide
CASRRRGLFETQYF

SEQ ID NO: 896 - protein, 14 aa, Artificial Sequence
  Description of Artificial Sequence Synthetic peptide
CASSPRLGPYEQYF

SEQ ID NO: 897 - protein, 12 aa, Artificial Sequence
  Description of Artificial Sequence Synthetic peptide
CASSPVLREQYF

SEQ ID NO: 898 - protein, 15 aa, Artificial Sequence
  Description of Artificial Sequence Synthetic peptide
CAMRELDNYGQNFVF

SEQ ID NO: 899 - protein, 15 aa, Artificial Sequence
  Description of Artificial Sequence Synthetic peptide
CAMSPSYNQGGKLIF

SEQ ID NO: 900 - protein, 14 aa, Artificial Sequence
  Description of Artificial Sequence Synthetic peptide
CALSSGAGSYQLTF

SEQ ID NO: 901 - protein, 14 aa, Artificial Sequence
  Description of Artificial Sequence Synthetic peptide
CAASKVQGAQKLVF

SEQ ID NO: 902 - protein, 14 aa, Artificial Sequence
  Description of Artificial Sequence Synthetic peptide
CASRDQYGTEKLFF

SEQ ID NO: 903 - protein, 13 aa, Artificial Sequence
  Description of Artificial Sequence Synthetic peptide
CASSESRYDEQYF

SEQ ID NO: 904 - protein, 14 aa, Artificial Sequence
  Description of Artificial Sequence Synthetic peptide
CSVEDRGVPYGYTF

SEQ ID NO: 905 - protein, 12 aa, Artificial Sequence
  Description of Artificial Sequence Synthetic peptide
CATSNHESEQYF

SEQ ID NO: 906 - protein, 13 aa, Artificial Sequence
  Description of Artificial Sequence Synthetic peptide
CASSYSGGNEQFF

SEQ ID NO: 907 - protein, 14 aa, Artificial Sequence
  Description of Artificial Sequence Synthetic peptide
CAVGDSSGTYKYIF

SEQ ID NO: 908 - protein, 11 aa, Artificial Sequence
  Description of Artificial Sequence Synthetic peptide
CAVDDGQKLLF

SEQ ID NO: 909 - protein, 12 aa, Artificial Sequence
  Description of Artificial Sequence Synthetic peptide
CAVNFGNEKLTF

SEQ ID NO: 910 - protein, 14 aa, Artificial Sequence
  Description of Artificial Sequence Synthetic peptide
CAVRRAGGGNKLTF

SEQ ID NO: 911 - protein, 14 aa, Artificial Sequence
  Description of Artificial Sequence Synthetic peptide
CAVRDRTGGFKTIF

SEQ ID NO: 912 - protein, 14 aa, Artificial Sequence
  Description of Artificial Sequence Synthetic peptide
CAVGSNTNAGKSTF

SEQ ID NO: 913 - protein, 16 aa, Artificial Sequence
  Description of Artificial Sequence Synthetic peptide
CASSPQALTANTEAFF

SEQ ID NO: 914 - protein, 12 aa, Artificial Sequence
  Description of Artificial Sequence Synthetic peptide
CASSRGLTEAFF

SEQ ID NO: 915 - protein, 15 aa, Artificial Sequence
  Description of Artificial Sequence Synthetic peptide
CASSYSGTLQETQYF

SEQ ID NO: 916 - protein, 14 aa, Artificial Sequence
  Description of Artificial Sequence Synthetic peptide
CASSIDRGLGEQYF

SEQ ID NO: 917 - protein, 13 aa, Artificial Sequence
  Description of Artificial Sequence Synthetic peptide
CASSQDRGTEAFF

SEQ ID NO: 918 - protein, 16 aa, Artificial Sequence
  Description of Artificial Sequence Synthetic peptide
CASSPQDRVAGDTIYF

SEQ ID NO: 919 - protein, 16 aa, Artificial Sequence
  Description of Artificial Sequence Synthetic peptide
CASSPGVAGVKETQYF

SEQ ID NO: 920 - protein, 18 aa, Artificial Sequence
  Description of Artificial Sequence Synthetic peptide
CATSRLGQLSTSTDTQYF

SEQ ID NO: 921 - protein, 14 aa, Artificial Sequence
  Description of Artificial Sequence Synthetic peptide
CASSQRQGGVEQYF

SEQ ID NO: 922 - protein, 13 aa, Artificial Sequence
  Description of Artificial Sequence Synthetic peptide
CASSGGWDQPQHF

SEQ ID NO: 923 - protein, 13 aa, Artificial Sequence
  Description of Artificial Sequence Synthetic peptide
CASSSTGFETQYF

SEQ ID NO: 924 - protein, 16 aa, Artificial Sequence
  Description of Artificial Sequence Synthetic peptide
CASSLLQGRPNEKLFF

SEQ ID NO: 925 - protein, 14 aa, Artificial Sequence
  Description of Artificial Sequence Synthetic peptide
CSVLTGTGLGGYTF

SEQ ID NO: 926 - protein, 15 aa, Artificial Sequence
  Description of Artificial Sequence Synthetic peptide
CASSLAGGRGDEQYF

SEQ ID NO: 927 - protein, 16 aa, Artificial Sequence
  Description of Artificial Sequence Synthetic peptide
CASSPRGAGVLNEQFF

SEQ ID NO: 928 - protein, 16 aa, Artificial Sequence
  Description of Artificial Sequence Synthetic peptide
CASSQAADRPTYEQYF

SEQ ID NO: 929 - protein, 16 aa, Artificial Sequence
  Description of Artificial Sequence Synthetic peptide
CASSWTNRGGNQPQHF

SEQ ID NO: 930 - protein, 17 aa, Artificial Sequence
  Description of Artificial Sequence Synthetic peptide
CASSLEYSGGKSGEQFF

SEQ ID NO: 931 - protein, 13 aa, Artificial Sequence
  Description of Artificial Sequence Synthetic peptide
CASSLGGNQPQHF

SEQ ID NO: 932 - protein, 14 aa, Artificial Sequence
  Description of Artificial Sequence Synthetic peptide
CASSSTGTNQPQHF

SEQ ID NO: 933 - protein, 13 aa, Artificial Sequence
  Description of Artificial Sequence Synthetic peptide
CATMSGAGNTIYF

SEQ ID NO: 934 - protein, 13 aa, Artificial Sequence
  Description of Artificial Sequence Synthetic peptide
CAVRDGGDYKLSF

SEQ ID NO: 935 - protein, 14 aa, Artificial Sequence
  Description of Artificial Sequence Synthetic peptide
CAAKISGSARQLTF

SEQ ID NO: 936 - protein, 11 aa, Artificial Sequence
  Description of Artificial Sequence Synthetic peptide
CASRNDYKLSF

SEQ ID NO: 937 - protein, 18 aa, Artificial Sequence
  Description of Artificial Sequence Synthetic peptide
CASSLAPRLSSSYNEQFF

SEQ ID NO: 938 - protein, 14 aa, Artificial Sequence
  Description of Artificial Sequence Synthetic peptide
CASIEGGTPGTQYF

SEQ ID NO: 939 - protein, 13 aa, Artificial Sequence
  Description of Artificial Sequence Synthetic peptide
CASSPAQGAGYTF

SEQ ID NO: 940 - protein, 15 aa, Artificial Sequence
  Description of Artificial Sequence Synthetic peptide
CASSLSVGAGETQYF

SEQ ID NO: 941 - protein, 17 aa, Artificial Sequence
  Description of Artificial Sequence Synthetic peptide
CALTRVFRGGSNYKLTF

SEQ ID NO: 942 - protein, 13 aa, Artificial Sequence
  Description of Artificial Sequence Synthetic peptide
CALRQGDNNDMRF

SEQ ID NO: 943 - protein, 15 aa, Artificial Sequence
  Description of Artificial Sequence Synthetic peptide
CSVYSIQSRANVLTF

SEQ ID NO: 944 - protein, 14 aa, Artificial Sequence
  Description of Artificial Sequence Synthetic peptide
CAARKTSGTYKYIF

SEQ ID NO: 945 - protein, 14 aa, Artificial Sequence
  Description of Artificial Sequence Synthetic peptide
CAARKTSGTYKYIF

SEQ ID NO: 946 - protein, 11 aa, Artificial Sequence
  Description of Artificial Sequence Synthetic peptide
CAVSSVDIPTF

SEQ ID NO: 947 - protein, 16 aa, Artificial Sequence
  Description of Artificial Sequence Synthetic peptide
CAPDPAGGTSYGKLTF

SEQ ID NO: 948 - protein, 14 aa, Artificial Sequence
  Description of Artificial Sequence Synthetic peptide
CILRGSDSNYQLIW

SEQ ID NO: 949 - protein, 15 aa, Artificial Sequence
  Description of Artificial Sequence Synthetic peptide
CASSLSAGANSPLHF

SEQ ID NO: 950 - protein, 15 aa, Artificial Sequence
  Description of Artificial Sequence Synthetic peptide
CASSTQTGTSDIQYF

SEQ ID NO: 951 - protein, 15 aa, Artificial Sequence
  Description of Artificial Sequence Synthetic peptide
CASSMTSGDTGELFF

SEQ ID NO: 952 - protein, 18 aa, Artificial Sequence
  Description of Artificial Sequence Synthetic peptide
CASSSPTRELYSNQPQHF

SEQ ID NO: 953 - protein, 15 aa, Artificial Sequence
  Description of Artificial Sequence Synthetic peptide
CASSQVSSGSYEQYF

SEQ ID NO: 954 - protein, 15 aa, Artificial Sequence
  Description of Artificial Sequence Synthetic peptide
CASSPTQDINQPQHF

SEQ ID NO: 955 - protein, 15 aa, Artificial Sequence
  Description of Artificial Sequence Synthetic peptide
CASSQDLRILNTQYF

SEQ ID NO: 956 - protein, 11 aa, Artificial Sequence
  Description of Artificial Sequence Synthetic peptide
CAPNQGGKLIF

SEQ ID NO: 957 - protein, 15 aa, Artificial Sequence
  Description of Artificial Sequence Synthetic peptide
CASSLLAGSNHEQYF

SEQ ID NO: 958 - protein, 14 aa, Artificial Sequence
  Description of Artificial Sequence Synthetic peptide
CASSQDLQKNIQYF

SEQ ID NO: 959 - protein, 14 aa, Artificial Sequence
  Description of Artificial Sequence Synthetic peptide
CASSWGRENTEAFF

SEQ ID NO: 960 - protein, 11 aa, Artificial Sequence
  Description of Artificial Sequence Synthetic peptide
CAMSFNNDMRF

SEQ ID NO: 961 - protein, 13 aa, Artificial Sequence
  Description of Artificial Sequence Synthetic peptide
CAMSDTGFQKLVF

SEQ ID NO: 962 - protein, 12 aa, Artificial Sequence
  Description of Artificial Sequence Synthetic peptide
CAMDTGFQKLVF

SEQ ID NO: 963 - protein, 16 aa, Artificial Sequence
  Description of Artificial Sequence Synthetic peptide
CAASPPGSGGYNKLIF

SEQ ID NO: 964 - protein, 15 aa, Artificial Sequence
  Description of Artificial Sequence Synthetic peptide
CAESDSGAGSYQLTF

SEQ ID NO: 965 - protein, 12 aa, Artificial Sequence
  Description of Artificial Sequence Synthetic peptide
CLVAPPDARLMF

SEQ ID NO: 966 - protein, 12 aa, Artificial Sequence
  Description of Artificial Sequence Synthetic peptide
CAERDPGNMLTF

SEQ ID NO: 967 - protein, 11 aa, Artificial Sequence
  Description of Artificial Sequence Synthetic peptide
CAEYSGYALNF

SEQ ID NO: 968 - protein, 14 aa, Artificial Sequence
  Description of Artificial Sequence Synthetic peptide
CAMRGGYAGNKLTF

SEQ ID NO: 969 - protein, 15 aa, Artificial Sequence
  Description of Artificial Sequence Synthetic peptide
CAMRGVYNQGGKLIF

SEQ ID NO: 970 - protein, 17 aa, Artificial Sequence
  Description of Artificial Sequence Synthetic peptide
CAMRVHNSGAGSYQLTF

SEQ ID NO: 971 - protein, 14 aa, Artificial Sequence
  Description of Artificial Sequence Synthetic peptide
CAMRGMNRDDKIIF

SEQ ID NO: 972 - protein, 13 aa, Artificial Sequence
  Description of Artificial Sequence Synthetic peptide
CALKIGSQGNLIF

SEQ ID NO: 973 - protein, 16 aa, Artificial Sequence
  Description of Artificial Sequence Synthetic peptide
CLVGDLYGGATNKLIF

SEQ ID NO: 974 - protein, 16 aa, Artificial Sequence
  Description of Artificial Sequence Synthetic peptide
CASSQEPGSVHNEQFF

SEQ ID NO: 975 - protein, 15 aa, Artificial Sequence
  Description of Artificial Sequence Synthetic peptide
CAASKGSSNTGKLIF

SEQ ID NO: 976 - protein, 15 aa, Artificial Sequence
  Description of Artificial Sequence Synthetic peptide
CAFMKPGGSNYKLTF

SEQ ID NO: 977 - protein, 12 aa, Artificial Sequence
  Description of Artificial Sequence Synthetic peptide
CAAAVKGNQFYF

SEQ ID NO: 978 - protein, 14 aa, Artificial Sequence
  Description of Artificial Sequence Synthetic peptide
CAVGLNTGFQKLVF

SEQ ID NO: 979 - protein, 12 aa, Artificial Sequence
  Description of Artificial Sequence Synthetic peptide
CAITGSNYQLIW

SEQ ID NO: 980 - protein, 15 aa, Artificial Sequence
  Description of Artificial Sequence Synthetic peptide
CALRLTQGGSEKLVF

SEQ ID NO: 981 - protein, 16 aa, Artificial Sequence
  Description of Artificial Sequence Synthetic peptide
CALSEYSGAGSYQLTF

SEQ ID NO: 982 - protein, 15 aa, Artificial Sequence
  Description of Artificial Sequence Synthetic peptide
CAASRVTGGGNKLTF

SEQ ID NO: 983 - protein, 16 aa, Artificial Sequence
  Description of Artificial Sequence Synthetic peptide
CIARSDMKTSYDKVIF

SEQ ID NO: 984 - protein, 13 aa, Artificial Sequence
  Description of Artificial Sequence Synthetic peptide
CIPLDDNDYKLSF

SEQ ID NO: 985 - protein, 15 aa, Artificial Sequence
  Description of Artificial Sequence Synthetic peptide
CIAQRAQGGSEKLVF

SEQ ID NO: 986 - protein, 14 aa, Artificial Sequence
  Description of Artificial Sequence Synthetic peptide
CAVWGSGGYQKVTF

SEQ ID NO: 987 - protein, 12 aa, Artificial Sequence
  Description of Artificial Sequence Synthetic peptide
CAVRENNARLMF

SEQ ID NO: 988 - protein, 14 aa, Artificial Sequence
  Description of Artificial Sequence Synthetic peptide
CASSPNNPGNTIYF

SEQ ID NO: 989 - protein, 14 aa, Artificial Sequence
  Description of Artificial Sequence Synthetic peptide
CASTPMALGTEAFF

SEQ ID NO: 990 - protein, 10 aa, Artificial Sequence
  Description of Artificial Sequence Synthetic peptide
CASSGQPQHF

SEQ ID NO: 991 - protein, 18 aa, Artificial Sequence
  Description of Artificial Sequence Synthetic peptide
CASSLAGTSGSGADTQYF

SEQ ID NO: 992 - protein, 16 aa, Artificial Sequence
  Description of Artificial Sequence Synthetic peptide
CASSNNRGRSNQPQHF

SEQ ID NO: 993 - protein, 16 aa, Artificial Sequence
  Description of Artificial Sequence Synthetic peptide
CASSSHVSRGRETQYF

SEQ ID NO: 994 - protein, 14 aa, Artificial Sequence
  Description of Artificial Sequence Synthetic peptide
CASKEKNRAYGYTF

SEQ ID NO: 995 - protein, 13 aa, Artificial Sequence
  Description of Artificial Sequence Synthetic peptide
CASSLGSGGEAFF

SEQ ID NO: 996 - protein, 17 aa, Artificial Sequence
  Description of Artificial Sequence Synthetic peptide
CASSVATGQGLEDTQYF

SEQ ID NO: 997 - protein, 14 aa, Artificial Sequence
  Description of Artificial Sequence Synthetic peptide
CASSLYAGNYGYTF

SEQ ID NO: 998 - protein, 15 aa, Artificial Sequence
  Description of Artificial Sequence Synthetic peptide
CAISGKLGGSYEQYF

SEQ ID NO: 999 - protein, 14 aa, Artificial Sequence
  Description of Artificial Sequence Synthetic peptide
CASSGGQIQETQYF

SEQ ID NO: 1000 - protein, 13 aa, Artificial Sequence
  Description of Artificial Sequence Synthetic peptide
CAWDGTGNTEAFF

SEQ ID NO: 1001 - protein, 12 aa, Artificial Sequence
  Description of Artificial Sequence Synthetic peptide
CAFIPTYDKVIF

SEQ ID NO: 1002 - protein, 12 aa, Artificial Sequence
  Description of Artificial Sequence Synthetic peptide
CAMDQRNEKLTF

SEQ ID NO: 1003 - protein, 13 aa, Artificial Sequence
  Description of Artificial Sequence Synthetic peptide
CATDDNYGQNFVF

SEQ ID NO: 1004 - protein, 17 aa, Artificial Sequence
  Description of Artificial Sequence Synthetic peptide
CATDAPDSGAGSYQLTF

SEQ ID NO: 1005 - protein, 16 aa, Artificial Sequence
  Description of Artificial Sequence Synthetic peptide
CASNGPAKTGANNLFF

SEQ ID NO: 1006 - protein, 13 aa, Artificial Sequence
  Description of Artificial Sequence Synthetic peptide
CATDAAGTYKYIF

SEQ ID NO: 1007 - protein, 16 aa, Artificial Sequence
  Description of Artificial Sequence Synthetic peptide
CATVQISNFGNEKLTF

SEQ ID NO: 1008 - protein, 15 aa, Artificial Sequence
  Description of Artificial Sequence Synthetic peptide
CALISGGTSYGKLTF

SEQ ID NO: 1009 - protein, 14 aa, Artificial Sequence
  Description of Artificial Sequence Synthetic peptide
CALSGFSSARQLTF

SEQ ID NO: 1010 - protein, 13 aa, Artificial Sequence
  Description of Artificial Sequence Synthetic peptide
CALEEDSNYQLIW

SEQ ID NO: 1011 - protein, 15 aa, Artificial Sequence
  Description of Artificial Sequence Synthetic peptide
CASSWTSGNRDEQFF

SEQ ID NO: 1012 - protein, 13 aa, Artificial Sequence
  Description of Artificial Sequence Synthetic peptide
CASSINSNTEAFF

SEQ ID NO: 1013 - protein, 15 aa, Artificial Sequence
  Description of Artificial Sequence Synthetic peptide
CASSLYDPGNTEAFF

SEQ ID NO: 1014 - protein, 11 aa, Artificial Sequence
  Description of Artificial Sequence Synthetic peptide
CASRTINEQFF

SEQ ID NO: 1015 - protein, 19 aa, Artificial Sequence
  Description of Artificial Sequence Synthetic peptide
CASSSGTSGRAASFNEQFF

SEQ ID NO: 1016 - protein, 14 aa, Artificial Sequence
  Description of Artificial Sequence Synthetic peptide
CASSAGQLQETQYF

SEQ ID NO: 1017 - protein, 15 aa, Artificial Sequence
  Description of Artificial Sequence Synthetic peptide
CASSPGLAGLNEQFF

SEQ ID NO: 1018 - protein, 13 aa, Artificial Sequence
  Description of Artificial Sequence Synthetic peptide
CAAGGTSYGKLTF

SEQ ID NO: 1019 - protein, 12 aa, Artificial Sequence
  Description of Artificial Sequence Synthetic peptide
CAVRDRDDKIIF

SEQ ID NO: 1020 - protein, 11 aa, Artificial Sequence
  Description of Artificial Sequence Synthetic peptide
CAVEDRNDMRF

SEQ ID NO: 1021 - protein, 15 aa, Artificial Sequence
  Description of Artificial Sequence Synthetic peptide
CAHDSYGGATNKLIF

SEQ ID NO: 1022 - protein, 3 aa, Artificial Sequence
  Description of Artificial Sequence Synthetic peptide
LFF

SEQ ID NO: 1023 - protein, 12 aa, Artificial Sequence
  Description of Artificial Sequence Synthetic peptide
CAVYLGSNQFYF

SEQ ID NO: 1024 - protein, 14 aa, Artificial Sequence
  Description of Artificial Sequence Synthetic peptide
CGTDLPFNDYKLSF

SEQ ID NO: 1025 - protein, 17 aa, Artificial Sequence
  Description of Artificial Sequence Synthetic peptide
CIVRIPFSSGSARQLTF

SEQ ID NO: 1026 - protein, 13 aa, Artificial Sequence
  Description of Artificial Sequence Synthetic peptide
CAATPSGNTPLVF

SEQ ID NO: 1027 - protein, 13 aa, Artificial Sequence
  Description of Artificial Sequence Synthetic peptide
CAPYGGATNKLIF

SEQ ID NO: 1028 - protein, 14 aa, Artificial Sequence
  Description of Artificial Sequence Synthetic peptide
CASLSGAGSYQLTF

SEQ ID NO: 1029 - protein, 12 aa, Artificial Sequence
  Description of Artificial Sequence Synthetic peptide
CASHFNKEKLFF

SEQ ID NO: 1030 - protein, 14 aa, Artificial Sequence
  Description of Artificial Sequence Synthetic peptide
CASSFTGEGQPQHF

SEQ ID NO: 1031 - protein, 14 aa, Artificial Sequence
  Description of Artificial Sequence Synthetic peptide
CASREDRASYEQYF

SEQ ID NO: 1032 - protein, 15 aa, Artificial Sequence
  Description of Artificial Sequence Synthetic peptide
CASSVGLADYNEQFF

SEQ ID NO: 1033 - protein, 16 aa, Artificial Sequence
  Description of Artificial Sequence Synthetic peptide
CASSPTIRASHYGYTF

SEQ ID NO: 1034 - protein, 14 aa, Artificial Sequence
  Description of Artificial Sequence Synthetic peptide
CALSEAGSARQLTF

SEQ ID NO: 1035 - protein, 17 aa, Artificial Sequence
  Description of Artificial Sequence Synthetic peptide
CALSGSYSGAGSYQLTF

SEQ ID NO: 1036 - protein, 13 aa, Artificial Sequence
  Description of Artificial Sequence Synthetic peptide
CALSVSGNTPLVF

SEQ ID NO: 1037 - protein, 15 aa, Artificial Sequence
  Description of Artificial Sequence Synthetic peptide
CALSEATSGTYKYIF

SEQ ID NO: 1038 - protein, 13 aa, Artificial Sequence
  Description of Artificial Sequence Synthetic peptide
CAGQAGFGNVLHC

SEQ ID NO: 1039 - protein, 16 aa, Artificial Sequence
  Description of Artificial Sequence Synthetic peptide
CALSEAEGGGADGLTF

SEQ ID NO: 1040 - protein, 15 aa, Artificial Sequence
  Description of Artificial Sequence Synthetic peptide
CASSNSGRGADTQYF

SEQ ID NO: 1041 - protein, 15 aa, Artificial Sequence
  Description of Artificial Sequence Synthetic peptide
CASSLIAGDTDTQYF

SEQ ID NO: 1042 - protein, 15 aa, Artificial Sequence
  Description of Artificial Sequence Synthetic peptide
CASLPSADSNQPQHF

SEQ ID NO: 1043 - protein, 14 aa, Artificial Sequence
  Description of Artificial Sequence Synthetic peptide
CASSFVGAKNIQYF

SEQ ID NO: 1044 - protein, 14 aa, Artificial Sequence
  Description of Artificial Sequence Synthetic peptide
CASSLGISDEKLFF

SEQ ID NO: 1045 - protein, 13 aa, Artificial Sequence
  Description of Artificial Sequence Synthetic peptide
CAASILGRNQFYF

SEQ ID NO: 1046 - protein, 16 aa, Artificial Sequence
  Description of Artificial Sequence Synthetic peptide
CIVRVPIYNQGGKLIF

SEQ ID NO: 1047 - protein, 14 aa, Artificial Sequence
  Description of Artificial Sequence Synthetic peptide
CAVKAPNTGNQFYF

SEQ ID NO: 1048 - protein, 15 aa, Artificial Sequence
  Description of Artificial Sequence Synthetic peptide
CAGQRDSGGYQKVTF

SEQ ID NO: 1049 - protein, 14 aa, Artificial Sequence
  Description of Artificial Sequence Synthetic peptide
CATSRGGGSYEQYF

SEQ ID NO: 1050 - protein, 15 aa, Artificial Sequence
  Description of Artificial Sequence Synthetic peptide
CASSLATGGMGELFF

SEQ ID NO: 1051 - protein, 14 aa, Artificial Sequence
  Description of Artificial Sequence Synthetic peptide
CASSQARNYNEQFF

SEQ ID NO: 1052 - protein, 14 aa, Artificial Sequence
  Description of Artificial Sequence Synthetic peptide
CASSQGGRGHIQYF

SEQ ID NO: 1053 - protein, 13 aa, Artificial Sequence
  Description of Artificial Sequence Synthetic peptide
CASSLIQGNEQFF

SEQ ID NO: 1054 - protein, 16 aa, Artificial Sequence
  Description of Artificial Sequence Synthetic peptide
CASNQGQGLPNQPQHF

SEQ ID NO: 1055 - protein, 14 aa, Artificial Sequence
  Description of Artificial Sequence Synthetic peptide
CASSLDTLYTEAFF

SEQ ID NO: 1056 - protein, 14 aa, Artificial Sequence
  Description of Artificial Sequence Synthetic peptide
CASRTQGWDDGYTF

SEQ ID NO: 1057 - protein, 11 aa, Artificial Sequence
  Description of Artificial Sequence Synthetic peptide
CASSYGYEQYF

SEQ ID NO: 1058 - protein, 11 aa, Artificial Sequence
  Description of Artificial Sequence Synthetic peptide
CASSQVETQYF

SEQ ID NO: 1059 - protein, 4 aa, Artificial Sequence
  Description of Artificial Sequence Synthetic peptide
GYTF

SEQ ID NO: 1060 - protein, 20 aa, Artificial Sequence
  Description of Artificial Sequence Synthetic peptide
CASSYETNYRGYNVNTEAFF

SEQ ID NO: 1061 - protein, 18 aa, Artificial Sequence
  Description of Artificial Sequence Synthetic peptide
CALSETSNSGGSNYKLTF

SEQ ID NO: 1062 - protein, 13 aa, Artificial Sequence
  Description of Artificial Sequence Synthetic peptide
CAVEEGGYQKVTF

SEQ ID NO: 1063 - protein, 13 aa, Artificial Sequence
  Description of Artificial Sequence Synthetic peptide
CAVRGGGYQKVTF

SEQ ID NO: 1064 - protein, 14 aa, Artificial Sequence
  Description of Artificial Sequence Synthetic peptide
CAVVSSGSARQLTF

SEQ ID NO: 1065 - protein, 15 aa, Artificial Sequence
  Description of Artificial Sequence Synthetic peptide
CAVRRDGGATNKLIF

SEQ ID NO: 1066 - protein, 14 aa, Artificial Sequence
  Description of Artificial Sequence Synthetic peptide
CAVSDPSGTYKYIF

SEQ ID NO: 1067 - protein, 14 aa, Artificial Sequence
  Description of Artificial Sequence Synthetic peptide
CAVSVSSGNTPLVF

SEQ ID NO: 1068 - protein, 12 aa, Artificial Sequence
  Description of Artificial Sequence Synthetic peptide
CAATSRGSRLTF

SEQ ID NO: 1069 - protein, 16 aa, Artificial Sequence
  Description of Artificial Sequence Synthetic peptide
CAVSLGSGGSNYKLTF

SEQ ID NO: 1070 - protein, 14 aa, Artificial Sequence
  Description of Artificial Sequence Synthetic peptide
CAVSDLGSGYALNF

SEQ ID NO: 1071 - protein, 14 aa, Artificial Sequence
  Description of Artificial Sequence Synthetic peptide
CAGLYGGSQGNLIF

SEQ ID NO: 1072 - protein, 14 aa, Artificial Sequence
  Description of Artificial Sequence Synthetic peptide
CAYRSARGAQKLVF

SEQ ID NO: 1073 - protein, 13 aa, Artificial Sequence
  Description of Artificial Sequence Synthetic peptide
CALDEGNNARLMF

SEQ ID NO: 1074 - protein, 15 aa, Artificial Sequence
  Description of Artificial Sequence Synthetic peptide
CAAKGDQTGANNLFF

SEQ ID NO: 1075 - protein, 13 aa, Artificial Sequence
  Description of Artificial Sequence Synthetic peptide
CAVLTNSGYALNF

SEQ ID NO: 1076 - protein, 15 aa, Artificial Sequence
  Description of Artificial Sequence Synthetic peptide
CASSLSNPGLRGYTF

SEQ ID NO: 1077 - protein, 13 aa, Artificial Sequence
  Description of Artificial Sequence Synthetic peptide
CASRGLTTYEQYF

SEQ ID NO: 1078 - protein, 15 aa, Artificial Sequence
  Description of Artificial Sequence Synthetic peptide
CASSLGAGGLETQYF

SEQ ID NO: 1079 - protein, 15 aa, Artificial Sequence
  Description of Artificial Sequence Synthetic peptide
CASSYPASGTFEQYF

SEQ ID NO: 1080 - protein, 11 aa, Artificial Sequence
  Description of Artificial Sequence Synthetic peptide
CASTSSGTQYF

SEQ ID NO: 1081 - protein, 12 aa, Artificial Sequence
  Description of Artificial Sequence Synthetic peptide
CAYTGNAGKSTF

SEQ ID NO: 1082 - protein, 14 aa, Artificial Sequence
  Description of Artificial Sequence Synthetic peptide
CAASELGSNYKLTF

SEQ ID NO: 1083 - protein, 14 aa, Artificial Sequence
  Description of Artificial Sequence Synthetic peptide
CIDTNSNSGYALNF

SEQ ID NO: 1084 - protein, 11 aa, Artificial Sequence
  Description of Artificial Sequence Synthetic peptide
CACAAGNKLTF

SEQ ID NO: 1085 - protein, 15 aa, Artificial Sequence
  Description of Artificial Sequence Synthetic peptide
CASSFTGDSQAPLHF

SEQ ID NO: 1086 - protein, 15 aa, Artificial Sequence
  Description of Artificial Sequence Synthetic peptide
CASSLAAGLTYEQYF

SEQ ID NO: 1087 - protein, 14 aa, Artificial Sequence
  Description of Artificial Sequence Synthetic peptide
CASSARWADTEAFF

SEQ ID NO: 1088 - protein, 13 aa, Artificial Sequence
  Description of Artificial Sequence Synthetic peptide
CASSLNAGTEAFF

SEQ ID NO: 1089 - protein, 11 aa, Artificial Sequence
  Description of Artificial Sequence Synthetic peptide
CSAKTGVRRYF

SEQ ID NO: 1090 - protein, 16 aa, Artificial Sequence
  Description of Artificial Sequence Synthetic peptide
CASSPPGTGANYGYTF

SEQ ID NO: 1091 - protein, 14 aa, Artificial Sequence
  Description of Artificial Sequence Synthetic peptide
CASSRTRGADTQYF

SEQ ID NO: 1092 - protein, 13 aa, Artificial Sequence
  Description of Artificial Sequence Synthetic peptide
CASSRTSIEKLFF

SEQ ID NO: 1093 - protein, 14 aa, Artificial Sequence
  Description of Artificial Sequence Synthetic peptide
CASSEPTSNSPLHF

SEQ ID NO: 1094 - protein, 12 aa, Artificial Sequence
  Description of Artificial Sequence Synthetic peptide
CASSNGRYRFTF

SEQ ID NO: 1095 - protein, 13 aa, Artificial Sequence
  Description of Artificial Sequence Synthetic peptide
CASSLAGRETQYF

SEQ ID NO: 1096 - protein, 15 aa, Artificial Sequence
  Description of Artificial Sequence Synthetic peptide
CASSIPQGSKTEQFF

SEQ ID NO: 1097 - protein, 12 aa, Artificial Sequence
  Description of Artificial Sequence Synthetic peptide
CASSSNQTPENF

SEQ ID NO: 1098 - protein, 16 aa, Artificial Sequence
  Description of Artificial Sequence Synthetic peptide
CASSQVSRYSGNTIYF

SEQ ID NO: 1099 - protein, 14 aa, Artificial Sequence
  Description of Artificial Sequence Synthetic peptide
CSVEFPSSYNEQFF

SEQ ID NO: 1100 - protein, 12 aa, Artificial Sequence
  Description of Artificial Sequence Synthetic peptide
CAVQGGSEKLVF

SEQ ID NO: 1101 - protein, 12 aa, Artificial Sequence
  Description of Artificial Sequence Synthetic peptide
CAVVIPPGKLIF

SEQ ID NO: 1102 - protein, 15 aa, Artificial Sequence
  Description of Artificial Sequence Synthetic peptide
CIVREPLYNDYKLSF

SEQ ID NO: 1103 - protein, 7 aa, Artificial Sequence
  Description of Artificial Sequence Synthetic peptide
CIVSLDI

SEQ ID NO: 1104 - protein, 13 aa, Artificial Sequence
  Description of Artificial Sequence Synthetic peptide
CGTEFSNTGKLIF

SEQ ID NO: 1105 - protein, 11 aa, Artificial Sequence
  Description of Artificial Sequence Synthetic peptide
CIVRVDGDMRF

SEQ ID NO: 1106 - protein, 14 aa, Artificial Sequence
  Description of Artificial Sequence Synthetic peptide
CASQDSNPTETQYF

SEQ ID NO: 1107 - protein, 17 aa, Artificial Sequence
  Description of Artificial Sequence Synthetic peptide
CASSYSTIGGGRETQYF

SEQ ID NO: 1108 - protein, 15 aa, Artificial Sequence
  Description of Artificial Sequence Synthetic peptide
CAIRSEGTGFYEQYF

SEQ ID NO: 1109 - protein, 16 aa, Artificial Sequence
  Description of Artificial Sequence Synthetic peptide
CATSDSRLAGGHEQFF

SEQ ID NO: 1110 - protein, 12 aa, Artificial Sequence
  Description of Artificial Sequence Synthetic peptide
CASRQGNTEAFF

SEQ ID NO: 1111 - protein, 16 aa, Artificial Sequence
  Description of Artificial Sequence Synthetic peptide
CAVRLISGGSNYKLTF

SEQ ID NO: 1112 - protein, 13 aa, Artificial Sequence
  Description of Artificial Sequence Synthetic peptide
CAMKVIGGNKLVF

SEQ ID NO: 1113 - protein, 15 aa, Artificial Sequence
  Description of Artificial Sequence Synthetic peptide
CAASGRKTSGSRLTF

SEQ ID NO: 1114 - protein, 14 aa, Artificial Sequence
  Description of Artificial Sequence Synthetic peptide
CAVSDQTGANNLFF

SEQ ID NO: 1115 - protein, 13 aa, Artificial Sequence
  Description of Artificial Sequence Synthetic peptide
CAASTSSGYALNF

SEQ ID NO: 1116 - protein, 14 aa, Artificial Sequence
  Description of Artificial Sequence Synthetic peptide
CAARPGQGAQKLVF

SEQ ID NO: 1117 - protein, 11 aa, Artificial Sequence
  Description of Artificial Sequence Synthetic peptide
CAVDATDKLIF

SEQ ID NO: 1118 - protein, 14 aa, Artificial Sequence
  Description of Artificial Sequence Synthetic peptide
CAMREGYTGNQFYF

SEQ ID NO: 1119 - protein, 13 aa, Artificial Sequence
  Description of Artificial Sequence Synthetic peptide
CASSRDGPETQYF

SEQ ID NO: 1120 - protein, 13 aa, Artificial Sequence
  Description of Artificial Sequence Synthetic peptide
CASSEALRQPQHF

SEQ ID NO: 1121 - protein, 13 aa, Artificial Sequence
  Description of Artificial Sequence Synthetic peptide
CAWSYMTGREQFF

SEQ ID NO: 1122 - protein, 15 aa, Artificial Sequence
  Description of Artificial Sequence Synthetic peptide
CASSPRPGGSYEQYF

SEQ ID NO: 1123 - protein, 15 aa, Artificial Sequence
  Description of Artificial Sequence Synthetic peptide
CASSLQPRSSYEQYF

SEQ ID NO: 1124 - protein, 13 aa, Artificial Sequence
  Description of Artificial Sequence Synthetic peptide
CATSRDPGYEQFF

SEQ ID NO: 1125 - protein, 17 aa, Artificial Sequence
  Description of Artificial Sequence Synthetic peptide
CASRRRTGRGVLYEQYF

SEQ ID NO: 1126 - protein, 16 aa, Artificial Sequence
  Description of Artificial Sequence Synthetic peptide
CASSRIGGSSGNTIYF

SEQ ID NO: 1127 - protein, 16 aa, Artificial Sequence
  Description of Artificial Sequence Synthetic peptide
CASSQAKDASNQPQHF

SEQ ID NO: 1128 - protein, 15 aa, Artificial Sequence
  Description of Artificial Sequence Synthetic peptide
CASSLGAGLTYEQYF

SEQ ID NO: 1129 - protein, 17 aa, Artificial Sequence
  Description of Artificial Sequence Synthetic peptide
CASSAGVGLAGEAEQFF

SEQ ID NO: 1130 - protein, 13 aa, Artificial Sequence
  Description of Artificial Sequence Synthetic peptide
CASSLGLTYEQYF

SEQ ID NO: 1131 - protein, 14 aa, Artificial Sequence
  Description of Artificial Sequence Synthetic peptide
CTILSNFGNEKLTF

SEQ ID NO: 1132 - protein, 14 aa, Artificial Sequence
  Description of Artificial Sequence Synthetic peptide
CAASSDDSWGKLQF

SEQ ID NO: 1133 - protein, 13 aa, Artificial Sequence
  Description of Artificial Sequence Synthetic peptide
CAASGNAGNMLTF

SEQ ID NO: 1134 - protein, 15 aa, Artificial Sequence
  Description of Artificial Sequence Synthetic peptide
CAASLIENSGYALNF

SEQ ID NO: 1135 - protein, 15 aa, Artificial Sequence
  Description of Artificial Sequence Synthetic peptide
CAASASKTSYDKVIF

SEQ ID NO: 1136 - protein, 13 aa, Artificial Sequence
  Description of Artificial Sequence Synthetic peptide
CAAQQGGSEKLVF

SEQ ID NO: 1137 - protein, 16 aa, Artificial Sequence
  Description of Artificial Sequence Synthetic peptide
CAVRDNSGAGSYQLTF

SEQ ID NO: 1138 - protein, 12 aa, Artificial Sequence
  Description of Artificial Sequence Synthetic peptide
CAVEGFGNVLHC

SEQ ID NO: 1139 - protein, 15 aa, Artificial Sequence
  Description of Artificial Sequence Synthetic peptide
CAVLSTQTGANNLFF

SEQ ID NO: 1140 - protein, 16 aa, Artificial Sequence
  Description of Artificial Sequence Synthetic peptide
CASNPIGQSNTGELFF

SEQ ID NO: 1141 - protein, 14 aa, Artificial Sequence
  Description of Artificial Sequence Synthetic peptide
CASSLGQGRQTQYF

SEQ ID NO: 1142 - protein, 18 aa, Artificial Sequence
  Description of Artificial Sequence Synthetic peptide
CASSQEFARASSLNEQFF

SEQ ID NO: 1143 - protein, 16 aa, Artificial Sequence
  Description of Artificial Sequence Synthetic peptide
CASSPPRGGGYNEQFF

SEQ ID NO: 1144 - protein, 13 aa, Artificial Sequence
  Description of Artificial Sequence Synthetic peptide
CASSQQGRVGYTF

SEQ ID NO: 1145 - protein, 15 aa, Artificial Sequence
  Description of Artificial Sequence Synthetic peptide
CAVRDPIRSQGNLIF

SEQ ID NO: 1146 - protein, 16 aa, Artificial Sequence
  Description of Artificial Sequence Synthetic peptide
CAISESMGGTYTEAFF

SEQ ID NO: 1147 - protein, 15 aa, Artificial Sequence
  Description of Artificial Sequence Synthetic peptide
CASSLFGRGANVLTF

SEQ ID NO: 1148 - protein, 13 aa, Artificial Sequence
  Description of Artificial Sequence Synthetic peptide
CAASAGDNNDMRF

SEQ ID NO: 1149 - protein, 15 aa, Artificial Sequence
  Description of Artificial Sequence Synthetic peptide
CAGYAGGTSYGKLTF

SEQ ID NO: 1150 - protein, 14 aa, Artificial Sequence
  Description of Artificial Sequence Synthetic peptide
CAVEDNAGGFKTIF

SEQ ID NO: 1151 - protein, 16 aa, Artificial Sequence
  Description of Artificial Sequence Synthetic peptide
CASSFSLATSYNEQFF

SEQ ID NO: 1152 - protein, 12 aa, Artificial Sequence
  Description of Artificial Sequence Synthetic peptide
CASSSGGIVAFF

SEQ ID NO: 1153 - protein, 13 aa, Artificial Sequence
  Description of Artificial Sequence Synthetic peptide
CASSPAGRYGYTF

SEQ ID NO: 1154 - protein, 15 aa, Artificial Sequence
  Description of Artificial Sequence Synthetic peptide
CASSSDLQVGNTIYF

SEQ ID NO: 1155 - protein, 14 aa, Artificial Sequence
  Description of Artificial Sequence Synthetic peptide
CAMSAAGGYQKVTF

SEQ ID NO: 1156 - protein, 14 aa, Artificial Sequence
  Description of Artificial Sequence Synthetic peptide
CAGLDSNSGYALNF

SEQ ID NO: 1157 - protein, 10 aa, Artificial Sequence
  Description of Artificial Sequence Synthetic peptide
CAVGYDKVIF

SEQ ID NO: 1158 - protein, 12 aa, Artificial Sequence
  Description of Artificial Sequence Synthetic peptide
CASSEVGGGYTF

SEQ ID NO: 1159 - protein, 15 aa, Artificial Sequence
  Description of Artificial Sequence Synthetic peptide
CASSPPDRGQETQYF

SEQ ID NO: 1160 - protein, 14 aa, Artificial Sequence
  Description of Artificial Sequence Synthetic peptide
CASSVAGTAYGYTF

SEQ ID NO: 1161 - protein, 14 aa, Artificial Sequence
  Description of Artificial Sequence Synthetic peptide
CAVEDLRGTYKYIF

SEQ ID NO: 1162 - protein, 15 aa, Artificial Sequence
  Description of Artificial Sequence Synthetic peptide
CAVRPSGGGADGLTF

SEQ ID NO: 1163 - protein, 13 aa, Artificial Sequence
  Description of Artificial Sequence Synthetic peptide
CASSQASGIEQFF

SEQ ID NO: 1164 - protein, 12 aa, Artificial Sequence
  Description of Artificial Sequence Synthetic peptide
CASNRDTGELFF

SEQ ID NO: 1165 - protein, 18 aa, Artificial Sequence
  Description of Artificial Sequence Synthetic peptide
CASSIGTGGLANTGELFF

SEQ ID NO: 1166 - protein, 15 aa, Artificial Sequence
  Description of Artificial Sequence Synthetic peptide
CASSPTRGMNTEAFF

SEQ ID NO: 1167 - protein, 15 aa, Artificial Sequence
  Description of Artificial Sequence Synthetic peptide
CSAREKTNSGNTIYF

SEQ ID NO: 1168 - protein, 16 aa, Artificial Sequence
  Description of Artificial Sequence Synthetic peptide
CASSRVIQQRKNIQYF

SEQ ID NO: 1169 - protein, 15 aa, Artificial Sequence
  Description of Artificial Sequence Synthetic peptide
CAVRDGMEYGNKLVF

SEQ ID NO: 1170 - protein, 13 aa, Artificial Sequence
  Description of Artificial Sequence Synthetic peptide
CAVVTLGNEKLTF

SEQ ID NO: 1171 - protein, 15 aa, Artificial Sequence
  Description of Artificial Sequence Synthetic peptide
CAVRIPSGGSYIPTF

SEQ ID NO: 1172 - protein, 13 aa, Artificial Sequence
  Description of Artificial Sequence Synthetic peptide
CASRPPGTGEAFF

SEQ ID NO: 1173 - protein, 13 aa, Artificial Sequence
  Description of Artificial Sequence Synthetic peptide
CARDGGSQGNLIF

SEQ ID NO: 1174 - protein, 14 aa, Artificial Sequence
  Description of Artificial Sequence Synthetic peptide
CASRGQGSYTEAFF

SEQ ID NO: 1175 - protein, 12 aa, Artificial Sequence
  Description of Artificial Sequence Synthetic peptide
CGTTSNDYKLSF

SEQ ID NO: 1176 - protein, 11 aa, Artificial Sequence
  Description of Artificial Sequence Synthetic peptide
CGTEGDDKIIF

SEQ ID NO: 1177 - protein, 13 aa, Artificial Sequence
  Description of Artificial Sequence Synthetic peptide
CGASPNTGNQFYF

SEQ ID NO: 1178 - protein, 13 aa, Artificial Sequence
  Description of Artificial Sequence Synthetic peptide
CAGPRSNDYKLSF

SEQ ID NO: 1179 - protein, 15 aa, Artificial Sequence
  Description of Artificial Sequence Synthetic peptide
CASQPSGSSYNEQFF

SEQ ID NO: 1180 - protein, 12 aa, Artificial Sequence
  Description of Artificial Sequence Synthetic peptide
CASSPTSLELFF

SEQ ID NO: 1181 - protein, 12 aa, Artificial Sequence
  Description of Artificial Sequence Synthetic peptide
CAMRRQGGKLIF

SEQ ID NO: 1182 - protein, 15 aa, Artificial Sequence
  Description of Artificial Sequence Synthetic peptide
CAVRDSNSGYSTLTF

SEQ ID NO: 1183 - protein, 15 aa, Artificial Sequence
  Description of Artificial Sequence Synthetic peptide
CAVRDRYQAGTALIF

SEQ ID NO: 1184 - protein, 16 aa, Artificial Sequence
  Description of Artificial Sequence Synthetic peptide
CASSGEGQGKEKTQYF

SEQ ID NO: 1185 - protein, 16 aa, Artificial Sequence
  Description of Artificial Sequence Synthetic peptide
CASSTPASGPTGELFF

SEQ ID NO: 1186 - protein, 15 aa, Artificial Sequence
  Description of Artificial Sequence Synthetic peptide
CAGPDSGGGADGLTF

SEQ ID NO: 1187 - protein, 10 aa, Artificial Sequence
  Description of Artificial Sequence Synthetic peptide
CAGTGNQFYF

SEQ ID NO: 1188 - protein, 15 aa, Artificial Sequence
  Description of Artificial Sequence Synthetic peptide
CAFMGVSGSARQLTF

SEQ ID NO: 1189 - protein, 13 aa, Artificial Sequence
  Description of Artificial Sequence Synthetic peptide
CASSLTGDNEQFF

SEQ ID NO: 1190 - protein, 14 aa, Artificial Sequence
  Description of Artificial Sequence Synthetic peptide
CASSGQANSYEQYF

SEQ ID NO: 1191 - protein, 20 aa, Artificial Sequence
  Description of Artificial Sequence Synthetic peptide
CASSQDQGTSGGPTRGTQYF

SEQ ID NO: 1192 - protein, 13 aa, Artificial Sequence
  Description of Artificial Sequence Synthetic peptide
CASGFSGNTEAFF

SEQ ID NO: 1193 - protein, 13 aa, Artificial Sequence
  Description of Artificial Sequence Synthetic peptide
CASSDAGYNEQFF

SEQ ID NO: 1194 - protein, 12 aa, Artificial Sequence
  Description of Artificial Sequence Synthetic peptide
CASGQGREQAFF

SEQ ID NO: 1195 - protein, 16 aa, Artificial Sequence
  Description of Artificial Sequence Synthetic peptide
CAVDSWGSTNAGKSTF

SEQ ID NO: 1196 - protein, 13 aa, Artificial Sequence
  Description of Artificial Sequence Synthetic peptide
CAVIPDSNYQLIW

SEQ ID NO: 1197 - protein, 11 aa, Artificial Sequence
  Description of Artificial Sequence Synthetic peptide
CAVLYGNKLVF

SEQ ID NO: 1198 - protein, 12 aa, Artificial Sequence
  Description of Artificial Sequence Synthetic peptide
CAGSDSNYQLIW

SEQ ID NO: 1199 - protein, 12 aa, Artificial Sequence
  Description of Artificial Sequence Synthetic peptide
CAGRSYGQNFVF

SEQ ID NO: 1200 - protein, 14 aa, Artificial Sequence
  Description of Artificial Sequence Synthetic peptide
CASSSVGGGYGYTF

SEQ ID NO: 1201 - protein, 16 aa, Artificial Sequence
  Description of Artificial Sequence Synthetic peptide
CASSLLGGTYNEKLFF

SEQ ID NO: 1202 - protein, 18 aa, Artificial Sequence
  Description of Artificial Sequence Synthetic peptide
CASSAFPSSGGAGETQYF

SEQ ID NO: 1203 - protein, 15 aa, Artificial Sequence
  Description of Artificial Sequence Synthetic peptide
CAENPFKTSGSRLTF

SEQ ID NO: 1204 - protein, 12 aa, Artificial Sequence
  Description of Artificial Sequence Synthetic peptide
CAVSVRGNKLVF

SEQ ID NO: 1205 - protein, 12 aa, Artificial Sequence
  Description of Artificial Sequence Synthetic peptide
CASSHPLREAFF

SEQ ID NO: 1206 - protein, 15 aa, Artificial Sequence
  Description of Artificial Sequence Synthetic peptide
CASSPSTGWGSPLHF

SEQ ID NO: 1207 - protein, 15 aa, Artificial Sequence
  Description of Artificial Sequence Synthetic peptide
CTSSVGLADYNEQFF

SEQ ID NO: 1208 - protein, 17 aa, Artificial Sequence
  Description of Artificial Sequence Synthetic peptide
CASRLAFQREGTDTQYF

SEQ ID NO: 1209 - protein, 13 aa, Artificial Sequence
  Description of Artificial Sequence Synthetic peptide
CASSLGQNTEAFF

SEQ ID NO: 1210 - protein, 13 aa, Artificial Sequence
  Description of Artificial Sequence Synthetic peptide
CASSLSGNEKLFF

SEQ ID NO: 1211 - protein, 14 aa, Artificial Sequence
  Description of Artificial Sequence Synthetic peptide
CASSPSGREETQYF

SEQ ID NO: 1212 - protein, 15 aa, Artificial Sequence
  Description of Artificial Sequence Synthetic peptide
CALGDDQTGANNLFF

SEQ ID NO: 1213 - protein, 16 aa, Artificial Sequence
  Description of Artificial Sequence Synthetic peptide
CAGPDAGGTSYGKLTF

SEQ ID NO: 1214 - protein, 13 aa, Artificial Sequence
  Description of Artificial Sequence Synthetic peptide
CACREGNTGKLIF

SEQ ID NO: 1215 - protein, 14 aa, Artificial Sequence
  Description of Artificial Sequence Synthetic peptide
CAVGTPYNNNDMRF

SEQ ID NO: 1216 - protein, 13 aa, Artificial Sequence
  Description of Artificial Sequence Synthetic peptide
CAYRSADGQKLLF

SEQ ID NO: 1217 - protein, 16 aa, Artificial Sequence
  Description of Artificial Sequence Synthetic peptide
CAYTGGDGGSQGNLIF

SEQ ID NO: 1218 - protein, 14 aa, Artificial Sequence
  Description of Artificial Sequence Synthetic peptide
CAYRSPLTGNQFYF

SEQ ID NO: 1219 - protein, 17 aa, Artificial Sequence
  Description of Artificial Sequence Synthetic peptide
CASSFDNRGPISYEQYF

SEQ ID NO: 1220 - protein, 15 aa, Artificial Sequence
  Description of Artificial Sequence Synthetic peptide
CASSNRGSSTDTQYF

SEQ ID NO: 1221 - protein, 13 aa, Artificial Sequence
  Description of Artificial Sequence Synthetic peptide
CASSFNNDNEQFF

SEQ ID NO: 1222 - protein, 14 aa, Artificial Sequence
  Description of Artificial Sequence Synthetic peptide
CASSLTDFRPEAFF

SEQ ID NO: 1223 - protein, 11 aa, Artificial Sequence
  Description of Artificial Sequence Synthetic peptide
CAASHDYKLSF

SEQ ID NO: 1224 - protein, 12 aa, Artificial Sequence
  Description of Artificial Sequence Synthetic peptide
CAMGGATNKLIF

SEQ ID NO: 1225 - protein, 12 aa, Artificial Sequence
  Description of Artificial Sequence Synthetic peptide
CAADNYGQNFVF

SEQ ID NO: 1226 - protein, 13 aa, Artificial Sequence
  Description of Artificial Sequence Synthetic peptide
CASGDRLNTEAFF

SEQ ID NO: 1227 - protein, 12 aa, Artificial Sequence
  Description of Artificial Sequence Synthetic peptide
CASSSLGGEAFF

SEQ ID NO: 1228 - protein, 14 aa, Artificial Sequence
  Description of Artificial Sequence Synthetic peptide
CAVDSTDSNYQLIW

SEQ ID NO: 1229 - protein, 13 aa, Artificial Sequence
  Description of Artificial Sequence Synthetic peptide
CAVDLDSNYQLIW

SEQ ID NO: 1230 - protein, 16 aa, Artificial Sequence
  Description of Artificial Sequence Synthetic peptide
CAVDPPPMEYGNKLVF

SEQ ID NO: 1231 - protein, 13 aa, Artificial Sequence
  Description of Artificial Sequence Synthetic peptide
CLVGERNFNKFYF

SEQ ID NO: 1232 - protein, 13 aa, Artificial Sequence
  Description of Artificial Sequence Synthetic peptide
CAEILPHNARLMF

SEQ ID NO: 1233 - protein, 16 aa, Artificial Sequence
  Description of Artificial Sequence Synthetic peptide
CAMREGVDSSASKIIF

SEQ ID NO: 1234 - protein, 14 aa, Artificial Sequence
  Description of Artificial Sequence Synthetic peptide
CAASSPGSQGNLIF

SEQ ID NO: 1235 - protein, 16 aa, Artificial Sequence
  Description of Artificial Sequence Synthetic peptide
CLVGDGWNTNAGKSTF

SEQ ID NO: 1236 - protein, 16 aa, Artificial Sequence
  Description of Artificial Sequence Synthetic peptide
CLVGADRYSSASKIIF

SEQ ID NO: 1237 - protein, 12 aa, Artificial Sequence
  Description of Artificial Sequence Synthetic peptide
CASSPLRGPQYF

SEQ ID NO: 1238 - protein, 13 aa, Artificial Sequence
  Description of Artificial Sequence Synthetic peptide
CASSQGRGVEQYF

SEQ ID NO: 1239 - protein, 14 aa, Artificial Sequence
  Description of Artificial Sequence Synthetic peptide
CASSPSAGGNIQYF

SEQ ID NO: 1240 - protein, 13 aa, Artificial Sequence
  Description of Artificial Sequence Synthetic peptide
CASSLEGDQPQHF

SEQ ID NO: 1241 - protein, 12 aa, Artificial Sequence
  Description of Artificial Sequence Synthetic peptide
CVVNKGNARLMF

SEQ ID NO: 1242 - protein, 7 aa, Artificial Sequence
  Description of Artificial Sequence Synthetic peptide
CASGPQF

SEQ ID NO: 1243 - protein, 19 aa, Artificial Sequence
  Description of Artificial Sequence Synthetic peptide
CASSQDGRAGAGYTGELFF

SEQ ID NO: 1244 - protein, 14 aa, Artificial Sequence
  Description of Artificial Sequence Synthetic peptide
CASSPGGANTEAFF

SEQ ID NO: 1245 - protein, 15 aa, Artificial Sequence
  Description of Artificial Sequence Synthetic peptide
CASSRTGYSNQPQHF

SEQ ID NO: 1246 - protein, 14 aa, Artificial Sequence
  Description of Artificial Sequence Synthetic peptide
CASSLVGSTGELFF

SEQ ID NO: 1247 - protein, 15 aa, Artificial Sequence
  Description of Artificial Sequence Synthetic peptide
CASSAKTDAYNEQFF

SEQ ID NO: 1248 - protein, 15 aa, Artificial Sequence
  Description of Artificial Sequence Synthetic peptide
CASSPPGTSGYEQYF

SEQ ID NO: 1249 - protein, 12 aa, Artificial Sequence
  Description of Artificial Sequence Synthetic peptide
CASRPDHYGYTF

SEQ ID NO: 1250 - protein, 13 aa, Artificial Sequence
  Description of Artificial Sequence Synthetic peptide
CSNVKGGNTEAFF

SEQ ID NO: 1251 - protein, 19 aa, Artificial Sequence
  Description of Artificial Sequence Synthetic peptide
CASSLERRGGRRWDNEQFF

SEQ ID NO: 1252 - protein, 13 aa, Artificial Sequence
  Description of Artificial Sequence Synthetic peptide
CATSAPLDYGYTF

SEQ ID NO: 1253 - protein, 18 aa, Artificial Sequence
  Description of Artificial Sequence Synthetic peptide
CASSLCLPIIHHNSPLHF

SEQ ID NO: 1254 - protein, 19 aa, Artificial Sequence
  Description of Artificial Sequence Synthetic peptide
CLVGDPYMNYGGSQGNLIF

SEQ ID NO: 1255 - protein, 11 aa, Artificial Sequence
  Description of Artificial Sequence Synthetic peptide
CLVAPNNDMRF

SEQ ID NO: 1256 - protein, 17 aa, Artificial Sequence
  Description of Artificial Sequence Synthetic peptide
CASSDGTGQNSGTEAFF

SEQ ID NO: 1257 - protein, 13 aa, Artificial Sequence
  Description of Artificial Sequence Synthetic peptide
CVVTNTGGFKTIF

SEQ ID NO: 1258 - protein, 12 aa, Artificial Sequence
  Description of Artificial Sequence Synthetic peptide
CLVGETGNQFYF

SEQ ID NO: 1259 - protein, 13 aa, Artificial Sequence
  Description of Artificial Sequence Synthetic peptide
CAEKPNRDDKIIF

SEQ ID NO: 1260 - protein, 13 aa, Artificial Sequence
  Description of Artificial Sequence Synthetic peptide
CAETPNRDDKIIF

SEQ ID NO: 1261 - protein, 13 aa, Artificial Sequence
  Description of Artificial Sequence Synthetic peptide
CARTSSNTGKLIF

SEQ ID NO: 1262 - protein, 14 aa, Artificial Sequence
  Description of Artificial Sequence Synthetic peptide
CIVRGFSDGQKLLF

SEQ ID NO: 1263 - protein, 13 aa, Artificial Sequence
  Description of Artificial Sequence Synthetic peptide
CAMPGLLGYGYTF

SEQ ID NO: 1264 - protein, 15 aa, Artificial Sequence
  Description of Artificial Sequence Synthetic peptide
CASTLNPGPGNTIYF

SEQ ID NO: 1265 - protein, 16 aa, Artificial Sequence
  Description of Artificial Sequence Synthetic peptide
CATINGGAPSFYGYTF

SEQ ID NO: 1266 - protein, 14 aa, Artificial Sequence
  Description of Artificial Sequence Synthetic peptide
CAVSRGQGAQKLVF

SEQ ID NO: 1267 - protein, 9 aa, Artificial Sequence
  Description of Artificial Sequence Synthetic peptide
CTNAGKSTF

SEQ ID NO: 1268 - protein, 15 aa, Artificial Sequence
  Description of Artificial Sequence Synthetic peptide
CVVSPMDTGRRALTF

SEQ ID NO: 1269 - protein, 15 aa, Artificial Sequence
  Description of Artificial Sequence Synthetic peptide
CASTYGQGEYNEQFF

SEQ ID NO: 1270 - protein, 15 aa, Artificial Sequence
  Description of Artificial Sequence Synthetic peptide
CASSSEEGSGNTIYF

SEQ ID NO: 1271 - protein, 12 aa, Artificial Sequence
  Description of Artificial Sequence Synthetic peptide
CALRDSGYALNF

SEQ ID NO: 1272 - protein, 11 aa, Artificial Sequence
  Description of Artificial Sequence Synthetic peptide
CAVTNDYKLSF

SEQ ID NO: 1273 - protein, 14 aa, Artificial Sequence
  Description of Artificial Sequence Synthetic peptide
CAVIPREYGNKLVF

SEQ ID NO: 1274 - protein, 14 aa, Artificial Sequence
  Description of Artificial Sequence Synthetic peptide
CSARGDRANSPLHF

SEQ ID NO: 1275 - protein, 12 aa, Artificial Sequence
  Description of Artificial Sequence Synthetic peptide
CASNDGTYEQYF

SEQ ID NO: 1276 - protein, 14 aa, Artificial Sequence
  Description of Artificial Sequence Synthetic peptide
CASSALAGPEWQFF

SEQ ID NO: 1277 - protein, 14 aa, Artificial Sequence
  Description of Artificial Sequence Synthetic peptide
CAMREGQTGNQFYF

SEQ ID NO: 1278 - protein, 14 aa, Artificial Sequence
  Description of Artificial Sequence Synthetic peptide
CLVGAGAAGNKLTF

SEQ ID NO: 1279 - protein, 14 aa, Artificial Sequence
  Description of Artificial Sequence Synthetic peptide
CAVEDRNAGNMLTF

SEQ ID NO: 1280 - protein, 13 aa, Artificial Sequence
  Description of Artificial Sequence Synthetic peptide
CASSPGTLYEQYF

SEQ ID NO: 1281 - protein, 15 aa, Artificial Sequence
  Description of Artificial Sequence Synthetic peptide
CASSFTGPGNSPLHF

SEQ ID NO: 1282 - protein, 14 aa, Artificial Sequence
  Description of Artificial Sequence Synthetic peptide
CASSLGRAQNIQYF

SEQ ID NO: 1283 - protein, 14 aa, Artificial Sequence
  Description of Artificial Sequence Synthetic peptide
CASSRSEAKNIQYF

SEQ ID NO: 1284 - protein, 10 aa, Artificial Sequence
  Description of Artificial Sequence Synthetic peptide
TGAGSYQLTF

SEQ ID NO: 1285 - protein, 14 aa, Artificial Sequence
  Description of Artificial Sequence Synthetic peptide
CALGELPGTYKYIF

SEQ ID NO: 1286 - protein, 14 aa, Artificial Sequence
  Description of Artificial Sequence Synthetic peptide
CASSVAANNQPQHF

SEQ ID NO: 1287 - protein, 15 aa, Artificial Sequence
  Description of Artificial Sequence Synthetic peptide
CASSEWGGVDQPQHF

SEQ ID NO: 1288 - protein, 15 aa, Artificial Sequence
  Description of Artificial Sequence Synthetic peptide
CASSLGPPVLDEQYF

SEQ ID NO: 1289 - protein, 15 aa, Artificial Sequence
  Description of Artificial Sequence Synthetic peptide
CASSPSRGSNQPQHF

SEQ ID NO: 1290 - protein, 15 aa, Artificial Sequence
  Description of Artificial Sequence Synthetic peptide
CVVSSASGGSYIPTF

SEQ ID NO: 1291 - protein, 14 aa, Artificial Sequence
  Description of Artificial Sequence Synthetic peptide
CASSNPGANTEAFF

SEQ ID NO: 1292 - protein, 15 aa, Artificial Sequence
  Description of Artificial Sequence Synthetic peptide
CSASSPTGRVSEQYF

SEQ ID NO: 1293 - protein, 17 aa, Artificial Sequence
  Description of Artificial Sequence Synthetic peptide
CASSVNSGGARPGELFF

SEQ ID NO: 1294 - protein, 14 aa, Artificial Sequence
  Description of Artificial Sequence Synthetic peptide
CASSGGTGGNTIYF

SEQ ID NO: 1295 - protein, 13 aa, Artificial Sequence
  Description of Artificial Sequence Synthetic peptide
CASSEGIGAEAFF

SEQ ID NO: 1296 - protein, 15 aa, Artificial Sequence
  Description of Artificial Sequence Synthetic peptide
CASSFGGSGANVLTF

SEQ ID NO: 1297 - protein, 14 aa, Artificial Sequence
  Description of Artificial Sequence Synthetic peptide
CASSYLGGGNEQFF

SEQ ID NO: 1298 - protein, 16 aa, Artificial Sequence
  Description of Artificial Sequence Synthetic peptide
CASSLASLSFSETQYF

SEQ ID NO: 1299 - protein, 15 aa, Artificial Sequence
  Description of Artificial Sequence Synthetic peptide
CASSLESANTGELFF

SEQ ID NO: 1300 - protein, 17 aa, Artificial Sequence
  Description of Artificial Sequence Synthetic peptide
CASSQAGGTSGYNEQFF

SEQ ID NO: 1301 - protein, 12 aa, Artificial Sequence
  Description of Artificial Sequence Synthetic peptide
CAVGARNTPLVF

SEQ ID NO: 1302 - protein, 16 aa, Artificial Sequence
  Description of Artificial Sequence Synthetic peptide
CAVGEGNTGTASKLTF

SEQ ID NO: 1303 - protein, 15 aa, Artificial Sequence
  Description of Artificial Sequence Synthetic peptide
CAISDGDRPYNEQFF

SEQ ID NO: 1304 - protein, 12 aa, Artificial Sequence
  Description of Artificial Sequence Synthetic peptide
CAVSDNTDKLIF

SEQ ID NO: 1305 - protein, 12 aa, Artificial Sequence
  Description of Artificial Sequence Synthetic peptide
CASSGSARQLTF

SEQ ID NO: 1306 - protein, 12 aa, Artificial Sequence
  Description of Artificial Sequence Synthetic peptide
CAVIVTGNQFYS

SEQ ID NO: 1307 - protein, 15 aa, Artificial Sequence
  Description of Artificial Sequence Synthetic peptide
CASRTPTGKYTEAFF

SEQ ID NO: 1308 - protein, 12 aa, Artificial Sequence
  Description of Artificial Sequence Synthetic peptide
CAVSGGYGKLTF

SEQ ID NO: 1309 - protein, 12 aa, Artificial Sequence
  Description of Artificial Sequence Synthetic peptide
CAVSPGNTPLVF

SEQ ID NO: 1310 - protein, 12 aa, Artificial Sequence
  Description of Artificial Sequence Synthetic peptide
CAVSEGNTPLVF

SEQ ID NO: 1311 - protein, 12 aa, Artificial Sequence
  Description of Artificial Sequence Synthetic peptide
CAVSERDDKIIF

SEQ ID NO: 1312 - protein, 10 aa, Artificial Sequence
  Description of Artificial Sequence Synthetic peptide
CAVNTDKLIF

SEQ ID NO: 1313 - protein, 15 aa, Artificial Sequence
  Description of Artificial Sequence Synthetic peptide
CASSLPGEGGRPQHF

SEQ ID NO: 1314 - protein, 13 aa, Artificial Sequence
  Description of Artificial Sequence Synthetic peptide
CSAREVGVETQYF

SEQ ID NO: 1315 - protein, 14 aa, Artificial Sequence
  Description of Artificial Sequence Synthetic peptide
CAVGVSSNTGKLIF

SEQ ID NO: 1316 - protein, 16 aa, Artificial Sequence
  Description of Artificial Sequence Synthetic peptide
CALSRAGGTSYGKLTF

SEQ ID NO: 1317 - protein, 13 aa, Artificial Sequence
  Description of Artificial Sequence Synthetic peptide
CAASKGSNYKLTF

SEQ ID NO: 1318 - protein, 14 aa, Artificial Sequence
  Description of Artificial Sequence Synthetic peptide
CAASASNSGYALNF

SEQ ID NO: 1319 - protein, 15 aa, Artificial Sequence
  Description of Artificial Sequence Synthetic peptide
CASSLEQARNNEQFF

SEQ ID NO: 1320 - protein, 15 aa, Artificial Sequence
  Description of Artificial Sequence Synthetic peptide
CASSLEGAGTNEQFF

SEQ ID NO: 1321 - protein, 14 aa, Artificial Sequence
  Description of Artificial Sequence Synthetic peptide
CASNDLNEPGELFF

SEQ ID NO: 1322 - protein, 13 aa, Artificial Sequence
  Description of Artificial Sequence Synthetic peptide
CASSLALDYGYTF

SEQ ID NO: 1323 - protein, 16 aa, Artificial Sequence
  Description of Artificial Sequence Synthetic peptide
CASSQDGGASGNTIYF

SEQ ID NO: 1324 - protein, 17 aa, Artificial Sequence
  Description of Artificial Sequence Synthetic peptide
CASSQEMGVATNEKLFF

SEQ ID NO: 1325 - protein, 16 aa, Artificial Sequence
  Description of Artificial Sequence Synthetic peptide
CASSVGLSDLLNEQFF

SEQ ID NO: 1326 - protein, 12 aa, Artificial Sequence
  Description of Artificial Sequence Synthetic peptide
CASSESYGEQFF

SEQ ID NO: 1327 - protein, 15 aa, Artificial Sequence
  Description of Artificial Sequence Synthetic peptide
CANTLSQQTWKHHIF

SEQ ID NO: 1328 - protein, 17 aa, Artificial Sequence
  Description of Artificial Sequence Synthetic peptide
CASSPWDPQFSGNTIYF

SEQ ID NO: 1329 - protein, 14 aa, Artificial Sequence
  Description of Artificial Sequence Synthetic peptide
CAVIGLAPVNMLTF

SEQ ID NO: 1330 - protein, 15 aa, Artificial Sequence
  Description of Artificial Sequence Synthetic peptide
CAVSSYSGSARQLTF

SEQ ID NO: 1331 - protein, 15 aa, Artificial Sequence
  Description of Artificial Sequence Synthetic peptide
CAVEVSYNQGGKLIF

SEQ ID NO: 1332 - protein, 14 aa, Artificial Sequence
  Description of Artificial Sequence Synthetic peptide
CASSPPTGTYGYTF

SEQ ID NO: 1333 - protein, 14 aa, Artificial Sequence
  Description of Artificial Sequence Synthetic peptide
CASEDQGYTGELFF

SEQ ID NO: 1334 - protein, 13 aa, Artificial Sequence
  Description of Artificial Sequence Synthetic peptide
CASSLDDNSPLHF

SEQ ID NO: 1335 - protein, 15 aa, Artificial Sequence
  Description of Artificial Sequence Synthetic peptide
CSARGTTVSTDTQYF

SEQ ID NO: 1336 - protein, 14 aa, Artificial Sequence
  Description of Artificial Sequence Synthetic peptide
CASKASGGVATQYF

SEQ ID NO: 1337 - protein, 12 aa, Artificial Sequence
  Description of Artificial Sequence Synthetic peptide
CASSQETGELFF

SEQ ID NO: 1338 - protein, 13 aa, Artificial Sequence
  Description of Artificial Sequence Synthetic peptide
CALSAGSNYQLIW

SEQ ID NO: 1339 - protein, 14 aa, Artificial Sequence
  Description of Artificial Sequence Synthetic peptide
CALFSGGGADGLTF

SEQ ID NO: 1340 - protein, 17 aa, Artificial Sequence
  Description of Artificial Sequence Synthetic peptide
CAISESMAGESYNEQFF

SEQ ID NO: 1341 - protein, 14 aa, Artificial Sequence
  Description of Artificial Sequence Synthetic peptide
CATYGQTGHYGYTF

SEQ ID NO: 1342 - protein, 14 aa, Artificial Sequence
  Description of Artificial Sequence Synthetic peptide
CATSPGGESYGYTF

SEQ ID NO: 1343 - protein, 15 aa, Artificial Sequence
  Description of Artificial Sequence Synthetic peptide
CASSQEDRAADTQYF

SEQ ID NO: 1344 - protein, 14 aa, Artificial Sequence
  Description of Artificial Sequence Synthetic peptide
CAMSARSGTYKYIF

SEQ ID NO: 1345 - protein, 13 aa, Artificial Sequence
  Description of Artificial Sequence Synthetic peptide
CAVRSDSNYQLIW

SEQ ID NO: 1346 - protein, 15 aa, Artificial Sequence
  Description of Artificial Sequence Synthetic peptide
CASSLIAGANYEQYF

SEQ ID NO: 1347 - protein, 15 aa, Artificial Sequence
  Description of Artificial Sequence Synthetic peptide
CALSDRGGSNYKLTF

SEQ ID NO: 1348 - protein, 14 aa, Artificial Sequence
  Description of Artificial Sequence Synthetic peptide
CATRRQGRTDTQYF

SEQ ID NO: 1349 - protein, 12 aa, Artificial Sequence
  Description of Artificial Sequence Synthetic peptide
CASRLEGSPLHF

SEQ ID NO: 1350 - protein, 13 aa, Artificial Sequence
  Description of Artificial Sequence Synthetic peptide
CASSGPLGSPLHF

SEQ ID NO: 1351 - protein, 19 aa, Artificial Sequence
  Description of Artificial Sequence Synthetic peptide
CASSLEPSGGRRGDNEQFF

SEQ ID NO: 1352 - protein, 12 aa, Artificial Sequence
  Description of Artificial Sequence Synthetic peptide
CAWSGAGPLVHF

SEQ ID NO: 1353 - protein, 19 aa, Artificial Sequence
  Description of Artificial Sequence Synthetic peptide
CASSVEPSGGRRWDNEQFF

SEQ ID NO: 1354 - protein, 19 aa, Artificial Sequence
  Description of Artificial Sequence Synthetic peptide
CARSWGPRGGRRWDNEQFF

SEQ ID NO: 1355 - protein, 13 aa, Artificial Sequence
  Description of Artificial Sequence Synthetic peptide
CASSLGEKDGYTF

SEQ ID NO: 1356 - protein, 16 aa, Artificial Sequence
  Description of Artificial Sequence Synthetic peptide
CASSHSGGAPYNEQFF

SEQ ID NO: 1357 - protein, 13 aa, Artificial Sequence
  Description of Artificial Sequence Synthetic peptide
CAPVPPLDYGYTF

SEQ ID NO: 1358 - protein, 14 aa, Artificial Sequence
  Description of Artificial Sequence Synthetic peptide
CASSFLGGGQPQHF

SEQ ID NO: 1359 - protein, 12 aa, Artificial Sequence
  Description of Artificial Sequence Synthetic peptide
CLVGKGFQKLVF

SEQ ID NO: 1360 - protein, 13 aa, Artificial Sequence
  Description of Artificial Sequence Synthetic peptide
CAVRPRGRGKLIF

SEQ ID NO: 1361 - protein, 15 aa, Artificial Sequence
  Description of Artificial Sequence Synthetic peptide
CAGQESTSGTYKYIF

SEQ ID NO: 1362 - protein, 15 aa, Artificial Sequence
  Description of Artificial Sequence Synthetic peptide
CAVSVMDTGRRALTF

SEQ ID NO: 1363 - protein, 15 aa, Artificial Sequence
  Description of Artificial Sequence Synthetic peptide
CASSPSQGAHYEQYF

SEQ ID NO: 1364 - protein, 9 aa, Homo sapiens
NTEPVKDPY

SEQ ID NO: 1365 - protein, 9 aa, Homo sapiens
YTYQMHGEY

SEQ ID NO: 1366 - protein, 11 aa, Homo sapiens
LLKIDSFELLY

SEQ ID NO: 1367 - protein, 10 aa, Homo sapiens
KIDSFELLYY

SEQ ID NO: 1368 - protein, 11 aa, Homo sapiens
LKIDSFELLYY

SEQ ID NO: 1369 - protein, 10 aa, Homo sapiens
WLEAVYCGCY

SEQ ID NO: 1370 - protein, 9 aa, Homo sapiens
WSVFSTSLY

SEQ ID NO: 1371 - protein, 9 aa, Homo sapiens
NLLTTCSTV

SEQ ID NO: 1372 - protein, 9 aa, Homo sapiens
FTVTVTEPL

SEQ ID NO: 1373 - protein, 9 aa, Homo sapiens
YLEAKADLV

SEQ ID NO: 1374 - protein, 10 aa, Homo sapiens
YLDQLNHILA

SEQ ID NO: 1375 - protein, 11 aa, Homo sapiens
FLQEPLQVFNV

SEQ ID NO: 1376 - protein, 9 aa, Homo sapiens
FLPRGTPAL

SEQ ID NO: 1377 - protein, 10 aa, Homo sapiens
RMAEHHSFWV

SEQ ID NO: 1378 - protein, 9 aa, Homo sapiens
HLLRIFCTI

SEQ ID NO: 1379 - protein, 10 aa, Homo sapiens
SLSEVDIPSI

SEQ ID NO: 1380 - protein, 10 aa, Homo sapiens
LILKGIFCTI

SEQ ID NO: 1381 - protein, 9 aa, Homo sapiens
STWGGFDEL

SEQ ID NO: 1382 - protein, 9 aa, Homo sapiens
TVTEPLLVK

SEQ ID NO: 1383 - protein, 9 aa, Homo sapiens
GLQTRAFWK

SEQ ID NO: 1384 - protein, 9 aa, Homo sapiens
ILLYKNKRK

SEQ ID NO: 1385 - protein, 10 aa, Homo sapiens
LLQGDTPVRK

SEQ ID NO: 1386 - protein, 9 aa, Homo sapiens
SLDWETPSK

SEQ ID NO: 1387 - protein, 9 aa, Homo sapiens
KEMRPARAK

SEQ ID NO: 1388 - protein, 10 aa, Homo sapiens
RAVTNHSVYY

SEQ ID NO: 1389 - protein, 10 aa, Homo sapiens
SVYYPSECSK

SEQ ID NO: 1390 - protein, 10 aa, Homo sapiens
QLFEGMKAFK

SEQ ID NO: 1391 - protein, 10 aa, Homo sapiens
ALGQLFEGMK

SEQ ID NO: 1392 - protein, 10 aa, Homo sapiens
KMFRKLHNSY

SEQ ID NO: 1393 - protein, 10 aa, Homo sapiens
RLQARPRLGR

SEQ ID NO: 1394 - protein, 10 aa, Homo sapiens
RIPYKVVARR

SEQ ID NO: 1395 - protein, 9 aa, Homo sapiens
KGFSIDSGK

SEQ ID NO: 1396 - protein, 10 aa, Homo sapiens
TVASSCSSPT

SEQ ID NO: 1397 - protein, 10 aa, Homo sapiens
YEQHNGVDGL

SEQ ID NO: 1398 - protein, 9 aa, Homo sapiens
ILKGIFCTI

SEQ ID NO: 1399 - protein, 9 aa, Homo sapiens
SGKSRPLPV

SEQ ID NO: 1400 - protein, 9 aa, Homo sapiens
QPSGKSRPL

SEQ ID NO: 1401 - protein, 10 aa, Homo sapiens
TQAPPPPERK

SEQ ID NO: 1402 - protein, 9 aa, Homo sapiens
SSDCRVSQI

SEQ ID NO: 1403 - protein, 14 aa, Artificial Sequence
  Description of Artificial Sequence Synthetic peptide
CASSSGGLYNEQFF

(19) United States
(12) Patent Application Publication
(10) Pub. No.: US 2022/0380937 A1
(43) Pub. Date: Dec. 1, 2022

(54) IDENTIFICATION OF SPLICING-DERIVED ANTIGENS FOR TREATING CANCER

(71) Applicant: THE REGENTS OF THE UNIVERSITY OF CALIFORNIA, Oakland, CA (US)

(72) Inventors: Yi XING, Los Angeles, CA (US); Robert PRINS, Los Angeles, CA (US); Yang PAN, Los Angeles, CA (US); Alexander H. LEE, Los Angeles, CA (US)

(73) Assignee: THE REGENTS OF THE UNIVERSITY OF CALIFORNIA, Oakland, CA (US)

(21) Appl. No.: 17/775,198

(22) PCT Filed: Nov. 6, 2020
(86) PCT No.: PCT/US2020/059476

§ 371 Date: May 6, 2022

Related U.S. Application Data
(60) Provisional application No. 62/932,751, filed on Nov. 8, 2019.
(60) Provisional application No. 62/934,914, filed on Nov. 13, 2019.

(51) Int. Cl. C40B 40/08; A61K 35/15; A61K 35/17; A61K 35/28; A61K 35/545; C07K 14/725; G16B 30/10

(52) U.S. Cl. CPC C40B 40/08; A61K 35/15; A61K 35/17; A61K 35/28; A61K 35/545; C07K 14/7051; G16B 30/10; A61K 38/00

(57) ABSTRACT

Methods and processes to identify neoplastic tissue antigens derived from alternative splicing (AS) are described, in accordance with various embodiments of the invention. Also described are novel tumor antigens that are useful as targets in various immunotherapeutic approaches to treating brain cancer as well as novel engineered T cell Receptors (TCRs) and chimeric antigen receptors (CARs) that target these antigenic peptides.

### BACKGROUND OF THE INVENTION

This application claims benefit of priority of U.S. Provisional Patent Application No. 62/934,914, filed Nov. 13, 2019 and U.S. Provisional Patent Application No. 62/932,751, filed Nov. 8, 2019, both of which are hereby incorporated by reference in their entireties.

This invention was made with government support under Grant Numbers CA211015 and CA233074, awarded by the National Institutes of Health. The government has certain rights in the invention.

### FIELD OF THE INVENTION

This invention relates to the field of cancer therapies.

### BACKGROUND

Cancer immunotherapy has gained tremendous momentum in the past decade. The clinical effectiveness of checkpoint inhibitors, such as neutralizing antibodies against PD-1 and CTLA-4, is thought to result from their ability to reactivate tumor-specific T cells. Meanwhile, adoptive cell therapies use genetically modified T-cell receptors (TCRs) or synthetic chimeric antigen receptor T cells (CAR-T) for tumor-specific antigen recognition. The finding that cancer cells express specific T-cell-reactive antigens has galvanized epitope discovery in recent years. Nevertheless, the identification of tumor antigens remains a major challenge. Although somatic mutation-derived antigens have been successfully targeted by cancer therapies, this approach remains largely ineffective for tumors with low or moderate mutation loads. Thus, there is a need in the art for the identification and characterization of novel tumor antigens that are useful targets in cancer immunotherapies.

### SUMMARY OF THE INVENTION

Methods and processes to identify neoplastic tissue antigens derived from alternative splicing (AS) are described, in accordance with various embodiments. Also described are novel tumor antigens that are useful as targets in various immunotherapeutic approaches to treating brain cancer as well as novel engineered T cell Receptors (TCRs) and chimeric antigen receptors (CARs) that target these antigenic peptides.

In several embodiments, RNA sequencing (RNA-seq) data derived from a neoplastic source (or a collection of neoplastic sources) are utilized to identify AS events. In a number of embodiments, neoplastic AS events are compared to AS events of non-neoplastic tissue such that AS events that are specific or increased in neoplastic tissue are identified. In some embodiments, neoplastic AS events are compared to AS events in similar neoplastic tissue such that recurrent AS events in neoplastic tissue are identified. Various processes to validate neoplastic AS events are performed, in accordance with some embodiments. Likewise, several embodiments utilize the identification of neoplastic AS events to synthesize peptides for use as an antigen of the neoplastic tissue.

Alternative splicing is a major cellular mechanism for generating expression complexity, especially in regulatory and functional aspects (e.g., two splice variants of the same gene can have different regulatory and functional properties). In addition, alternative splicing contributes to the diversity of phenotypes in eukaryotic cells of an organism, where each cell has the same DNA genotype. In neoplastic cells, alternative splicing mechanisms can be dysregulated, leading to aberrant expression of various isoforms and formation of neoplasm antigens. Various embodiments are directed towards identifying dysregulated isoforms and neoplasm antigens, which can be utilized in a number of applications. For instance, identified AS events can be utilized to develop peptides that are encoded by nucleotides that span the splice junction and these peptides may be utilized to develop various cancer treatments.

Aspects of the disclosure relate to an engineered T-cell Receptor (TCR) comprising: a TCR alpha (TCR-a) CDR3 comprising an amino acid sequence with at least 90% sequence identity to SEQ ID NO:30 and a TCR beta (TCR-b) CDR3 comprising an amino acid sequence with at least 90% sequence identity to SEQ ID NO:31; a TCR alpha (TCR-a) CDR3 comprising an amino acid sequence with at least 90% sequence identity to SEQ ID NO:32 and a TCR beta (TCR-b) CDR3 comprising an amino acid sequence with at least 90% sequence identity to SEQ ID NO:33; a TCR alpha (TCR-a) CDR3 comprising an amino acid sequence with at least 90% sequence identity to SEQ ID NO:34 and a TCR beta (TCR-b) CDR3 comprising an amino acid sequence with at least 90% sequence identity to SEQ ID NO:35; a TCR alpha (TCR-a) CDR3 comprising an amino acid sequence with at least 90% sequence identity to SEQ ID NO:36 and a TCR beta (TCR-b) CDR3 comprising an amino acid sequence with at least 90% sequence identity to SEQ ID NO:37; a TCR alpha (TCR-a) CDR3 comprising an amino acid sequence with at least 90% sequence identity to SEQ ID NO:38 and a TCR beta (TCR-b) CDR3 comprising an amino acid sequence with at least 90% sequence identity to SEQ ID NO:39; a TCR alpha (TCR-a) CDR3 comprising an amino acid sequence with at least 90% sequence identity to SEQ ID NO:40 and a TCR beta (TCR-b) CDR3 comprising an amino acid sequence with at least 90% sequence identity to SEQ ID NO:41; a TCR alpha (TCR-a) CDR3 comprising an amino acid sequence with at least 90% sequence identity to SEQ ID NO:42 and a TCR beta (TCR-b) CDR3 comprising an amino acid sequence with at least 90% sequence identity to SEQ ID NO:43 or a TCR-a and TCR-b CDR3 comprising an amino acid sequence with at least 90% sequence identity to a TCR-a and TCR-b CDR3 pair from a clonotype listed in Table 6.

Further aspects relate to one or more nucleic acids encoding a TCR, CAR, or peptide of the disclosure. Certain aspects relate to a nucleic acid encoding a TCR-alpha and/or TCR-beta polypeptide. Also provided are nucleic acid vector(s) comprising the nucleic acid(s) of the disclosure. Further aspects relate to a cell, such as a therapeutic cell or a host cell, comprising a TCR, CAR, nucleic acid, or vector of the disclosure. Also provided are compositions comprising the cells, nucleic acids, or peptides of the disclosure. Yet further aspects relate to an in vitro isolated dendritic cell comprising a peptide, nucleic acid, or expression vector of the disclosure. Further aspects relate to an in vitro composition comprising a dendritic cell and a peptide of the disclosure. Further aspects relate to an engineered T-cell Receptor (TCR) or chimeric antigen receptor (CAR) that specifically recognizes a peptide of the disclosure. Aspects of the disclosure also relate to an antibody or antigen binding fragment thereof that specifically recognizes and binds to a peptide of the disclosure.

Further aspects of the disclosure relate to a method comprising transferring a nucleic acid of the disclosure into a cell. Further method aspects of the disclosure relate to a method for stimulating an immune response or for treating brain cancer comprising administering a composition, peptide, antibody, therapeutic cell, CAR, or TCR of the disclosure to a subject. Other method aspects of the disclosure relate to an in vitro method for making a dendritic cell vaccine comprising contacting a dendritic cell in vitro with a peptide of the disclosure. Other method aspects relate to a method of treating a subject for brain cancer comprising administering a peptide, composition, dendritic cell, antibody or antigen binding fragment, or cell of the disclosure.

Further aspects relate to: a peptide from the TRIM11 protein comprising at least 6 contiguous amino acids from the TRIM11 and comprising the amino acids QD, which correspond to the amino acids at positions 168-169 of SEQ ID NO:1; a peptide from the RCOR3 protein comprising at least 6 contiguous amino acids from the RCOR3 and comprising the amino acids QG, which correspond to the amino acids at positions 358-359 of SEQ ID NO:2; a peptide from the FAM76B protein comprising at least 6 contiguous amino acids from the FAM76B and comprising the amino acids DS, which correspond to the amino acids at positions 230-231 of SEQ ID NO:3; a peptide from the SLMAP protein comprising at least 6 contiguous amino acids from the SLMAP and comprising the amino acids NP, which correspond to the amino acids at positions 332-333 of SEQ ID NO:4; a peptide from the TMEM62 protein comprising at least 6 contiguous amino acids from the TMEM62 and comprising the amino acids LG, which correspond to the amino acids at positions 495-496 of SEQ ID NO:5; and/or a peptide from the PLA2G6 protein comprising at least 6 contiguous amino acids from the PLA2G6 and comprising the amino acids RL, which correspond to the amino acids at positions 395-396 of SEQ ID NO:6. Further aspects relate to a peptide comprising at least 6 contiguous amino acids from a peptide of Table la, Table lb, Table lc, or 4, wherein the peptide comprises an alternative splice site junction. Yet further aspects relate to a peptide comprising at least 6 contiguous amino acids encoded by an alternatively spliced nucleic acid, wherein the at least 6 contiguous amino acids are encoded on a nucleic acid that comprises an alternative splice site junction, and wherein the alternative splice site junction is an AS event selected from an AS event in Table 3a or 3b. An alternative splice site junction in a polypeptide refers to the amino acids that are encoded by the region of the mRNA that spans the alternative splice site. An alternative splice site in a nucleic acid refers to the nucleic acid residues that span the alternative splice site.

Also provided is a method of activating or expanding peptide-specific T cells comprising contacting a starting population of T cells from a mammalian subject and preferably from a blood sample from the mammalian subject cells ex vivo with the peptide of disclosure thereby activating, stimulating proliferation, and/or expanding peptide-specific T cells in the starting population. Further aspects relate to a peptide-specific T cell activated or expanded according to a method of the disclosure. Also provided are pharmaceutical compositions comprising the peptide-specific T cells activated or expanded according to a method of the disclosure.

In some embodiments, contacting is further defined as co-culturing the starting population of T cells with antigen presenting cells (APCs), wherein the APCs can present the peptide of the disclosure on their surface. In some embodiments, the APCs are dendritic cells. In some embodiments, the dendritic cells are autologous dendritic cells obtained from the mammalian subject. In some embodiments, contacting is further defined as co-culturing the starting population of T cells with artificial antigen presenting cells (aAPCs). In some embodiments, the artificial antigen presenting cells (aAPCs) comprise or consist of poly(lactide-co-glycolide) (PLGA), K562 cells, paramagnetic beads coated with CD3 and CD28 agonist antibodies, beads or microparticles coupled with an HLA-dimer and anti-CD28, or nanosize-aAPCs (nano-aAPC) that are preferably less than 100 nm in diameter. In some embodiments, the T cells are CD8+ T cells or CD4+ T cells. In some embodiments, the T cells are cytotoxic T lymphocytes (CTLs). In some embodiments, the starting population of cells comprises or consists of peripheral blood mononuclear cells (PBMCs). In some embodiments, the method further comprises isolating or purifying the T cells from the peripheral blood mononuclear cells (PBMCs). In some embodiments, the mammalian subject is a human. In some embodiments, the method further comprises reinfusing or administering the activated or expanded peptide-specific T cells to the subject. Further aspects relate to a peptide-specific T cell activated or expanded according to a method of the disclosure. Also provided are pharmaceutical compositions comprising the peptide-specific T cells activated or expanded according to a method of the disclosure.

In some embodiments, the AS event is selected from an AS event in Table 3a. In some embodiments, the AS event is selected from an AS event in Table 3b. In some embodiments, the disclosure relates to a CAR that targets a peptide of the disclosure, wherein the peptide comprises an AS event from table 3b. In some embodiments, the disclosure relates to a TCR that targets a peptide of the disclosure, wherein the peptide comprises an AS event from table 3a.

In some embodiments, the TCR comprises: engineered T-cell Receptor (TCR) comprising: a TCR alpha (TCR-a) CDR3 comprising an amino acid sequence with at least 70, 80, 85, 86, 87, 88, 89, 90, 91, 92, 93, 94, 95, 96 97, 98, 99, or 100% sequence identity to SEQ ID NO:30 and a TCR beta (TCR-b) CDR3 comprising an amino acid sequence with at least 70, 80, 85, 86, 87, 88, 89, 90, 91, 92, 93, 94, 95, 96 97, 98, 99, or 100% sequence identity to SEQ ID NO:31; a TCR alpha (TCR-a) CDR3 comprising an amino acid sequence with at least 70, 80, 85, 86, 87, 88, 89, 90, 91, 92, 93, 94, 95, 96 97, 98, 99, or 100% sequence identity to SEQ ID NO:32 and a TCR beta (TCR-b) CDR3 comprising an amino acid sequence with at least 70, 80, 85, 86, 87, 88, 89, 90, 91, 92, 93, 94, 95, 96 97, 98, 99, or 100% sequence identity to SEQ ID NO:33; a TCR alpha (TCR-a) CDR3 comprising an amino acid sequence with at least 70, 80, 85, 86, 87, 88, 89, 90, 91, 92, 93, 94, 95, 96 97, 98, 99, or 100% sequence identity to SEQ ID NO:34 and a TCR beta (TCR-b) CDR3 comprising an amino acid sequence with at least 70, 80, 85, 86, 87, 88, 89, 90, 91, 92, 93, 94, 95, 96 97, 98, 99, or 100% sequence identity to SEQ ID NO:35; a TCR alpha (TCR-a) CDR3 comprising an amino acid sequence with at least 70, 80, 85, 86, 87, 88, 89, 90, 91, 92, 93, 94, 95, 96 97, 98, 99, or 100% sequence identity to SEQ ID NO:36 and a TCR beta (TCR-b) CDR3 comprising an amino acid sequence with at least 70, 80, 85, 86, 87, 88, 89, 90, 91, 92, 93, 94, 95, 96 97, 98, 99, or 100% sequence identity to SEQ ID NO:37; a TCR alpha (TCR-a) CDR3 comprising an amino acid sequence with at least 70, 80, 85, 86, 87, 88, 89, 90, 91, 92, 93, 94, 95, 96 97, 98, 99, or 100% sequence identity to SEQ ID NO:38 and a TCR beta (TCR-b) CDR3 comprising an amino acid sequence with at least 70, 80, 85, 86, 87, 88, 89, 90, 91, 92, 93, 94, 95, 96 97, 98, 99, or 100% sequence identity to SEQ ID NO:39; a TCR alpha (TCR-a) CDR3 comprising an amino acid sequence with at least 70, 80, 85, 86, 87, 88, 89, 90, 91, 92, 93, 94, 95, 96 97, 98, 99, or 100% sequence identity to SEQ ID NO:40 and a TCR beta (TCR-b) CDR3 comprising an amino acid sequence with at least 70, 80, 85, 86, 87, 88, 89, 90, 91, 92, 93, 94, 95, 96 97, 98, 99, or 100% sequence identity to SEQ ID NO:41; or a TCR alpha (TCR-a) CDR3 comprising an amino acid sequence with at least 70, 80, 85, 86, 87, 88, 89, 90, 91, 92, 93, 94, 95, 96 97, 98, 99, or 100% sequence identity to SEQ ID NO:42 and a TCR beta (TCR-b) CDR3 comprising an amino acid sequence with at least 90% sequence identity to SEQ ID NO:43.

In some embodiments, the TCR comprises: a TCR alpha (TCR-a) variable region comprising an amino acid sequence with at least 80% sequence identity to SEQ ID NO:44 and a TCR beta (TCR-b) variable region comprising an amino acid sequence with at least 80% sequence identity to SEQ ID NO:45; a TCR alpha (TCR-a) variable region comprising an amino acid sequence with at least 80% sequence identity to SEQ ID NO:46 and a TCR beta (TCR-b) variable region comprising an amino acid sequence with at least 80% sequence identity to SEQ ID NO:47; or a TCR alpha (TCR-a) variable region comprising an amino acid sequence with at least 80% sequence identity to SEQ ID NO:48 and a TCR beta (TCR-b) variable region comprising an amino acid sequence with at least 80% sequence identity to SEQ ID NO:49.

In some embodiments, the TCR comprises: a TCR alpha (TCR-a) variable region comprising an amino acid sequence with at least 70, 80, 85, 86, 87, 88, 89, 90, 91, 92, 93, 94, 95, 96 97, 98, 99, or 100% sequence identity to SEQ ID NO:44 and a TCR beta (TCR-b) variable region comprising an amino acid sequence with at least 70, 80, 85, 86, 87, 88, 89, 90, 91, 92, 93, 94, 95, 96 97, 98, 99, or 100% sequence identity to SEQ ID NO:45; a TCR alpha (TCR-a) variable region comprising an amino acid sequence with at least 70, 80, 85, 86, 87, 88, 89, 90, 91, 92, 93, 94, 95, 96 97, 98, 99, or 100% sequence identity to SEQ ID NO:46 and a TCR beta (TCR-b) variable region comprising an amino acid sequence with at least 70, 80, 85, 86, 87, 88, 89, 90, 91, 92, 93, 94, 95, 96 97, 98, 99, or 100% sequence identity to SEQ ID NO:47; or a TCR alpha (TCR-a) variable region comprising an amino acid sequence with at least 70, 80, 85, 86, 87, 88, 89, 90, 91, 92, 93, 94, 95, 96 97, 98, 99, or 100% sequence identity to SEQ ID NO:48 and a TCR beta (TCR-b) variable region comprising an amino acid sequence with at least 70, 80, 85, 86, 87, 88, 89, 90, 91, 92, 93, 94, 95, 96 97, 98, 99, or 100% sequence identity to SEQ ID NO:49.

In some embodiments, the TCR comprises or consists of a bispecific TCR. The bispecific TCR may comprises an scFv that targets or selectively binds CD3. In some embodiments, the TCR is further defined as a single-chain TCR (scTCR), wherein the a chain and the β chain are covalently attached via a flexible linker.In some embodiments, the TCR comprises a modification or is chimeric. In some embodiments, the variable region of the TCR is fused to a TCR constant region that is different from the constant region of the cloned TCR that specifically binds to a peptide of the disclosure.

In some embodiments, the nucleic acid of the disclosure comprises a cDNA encoding the TCR. In some embodiments, the TCR alpha and beta genes are on the same nucleic acid and/or on the same vector.

In some embodiments, a cell of the disclosure comprises an immune cell. In some embodiments, a cell of the disclosure comprises stem cell, progenitor cell, T cell, NK cell, invariant NK cell, NKT cell, mesenchymal stem cell (MSC), induced pluripotent stem (iPS) cell, regulatory T cell, CD8+ T cell, CD4+ T cell, or γδ T cell. In some embodiments, the cell comprises a hematopoietic stem or progenitor cell, a T cell, or an induced pluripotent stem cell (iPSC). In some embodiments, the cell is isolated from a cancer patient. In some embodiments, is a HLA-A type. The cell of the disclosure may be autologous or allogeneic. In some embodiments, the cell is a HLA-A*03:01, HLA-A*01:01, or HLA-A*02:01 type. In some embodiments, the cell comprises at least one TCR and at least one CAR and wherein the TCR and CAR each recognize a different peptide. For example, embodiments of the disclosure relate to a cell that comprises a TCR that targets one peptide of the disclosure and a CAR that targets a different peptide of the disclosure.

In some embodiments, the composition of the disclosure has been determined to be serum-free, mycoplasma-free, endotoxin-free, and/or sterile.

In some embodiments, the method further comprises culturing the cell in media, incubating the cell at conditions that allow for the division of the cell, screening the cell, and/or freezing the cell. In some embodiments, the method further comprises isolating the expressed peptide or polypeptide from a cell of the disclosure.

In some embodiments, the brain cancer comprises glioblastoma or glioma. In some embodiments, the subject has previously been treated for the cancer. In some embodiments, the subject has been determined to be resistant to the previous treatment. In some embodiments, the method further comprises the administration of an additional therapy. In some embodiments, the additional therapy comprises an immunotherapy, chemotherapy, or an additional therapy described herein. In some embodiments, the cancer comprises stage I, II, III, or IV cancer. In some embodiments, the cancer comprises metastatic and/or recurrent cancer.

In some embodiments, a peptide of the disclosure comprises at least 6 contiguous amino acids from one of SEQ ID NOS:786 or 1364-1395. In some embodiments, a peptide of the disclosure has at least 70% sequence identity to a peptide of SEQ ID NO:786 or 1364-1395. In some embodiments, a peptide of the disclosure has at least 80, 85, 86, 87, 88, 89, 90, 91, 92, 93, 94, 95, 96, 97, 98, or 99% sequence identity to SEQ ID NOS:786 or 1364-1395.

In some embodiments, the peptide comprises an amino acid sequence selected from SEQ ID NO:7-9. In some embodiments, the peptide comprises an amino acid sequence with at least 80, 85, 86, 87, 88, 89, 90, 91, 92, 93, 94, 95, 96, 97, 98, or 99% sequence identity to SEQ ID NO:7-9. In some embodiments, the peptide comprises an amino acid sequence of SEQ ID NO:10. In some embodiments, the peptide comprises an amino acid sequence with at least 80, 85, 86, 87, 88, 89, 90, 91, 92, 93, 94, 95, 96, 97, 98, or 99% sequence identity to SEQ ID NO:10. In some embodiments, the peptide comprises an amino acid sequence of SEQ ID NO:11 or 12. In some embodiments, the peptide comprises an amino acid sequence with at least 80, 85, 86, 87, 88, 89, 90, 91, 92, 93, 94, 95, 96, 97, 98, or 99% sequence identity to SEQ ID NO:11 or 12. In some embodiments, the peptide comprises an amino acid sequence selected from SEQ ID NO:13-15. In some embodiments, the peptide comprises an amino acid sequence with at least 80, 85, 86, 87, 88, 89, 90, 91, 92, 93, 94, 95, 96, 97, 98, or 99% sequence identity to SEQ ID NO:13-15. In some embodiments, the peptide comprises an amino acid sequence selected from SEQ ID NO:16-22. In some embodiments, the peptide comprises an amino acid sequence with at least 80, 85, 86, 87, 88, 89, 90, 91, 92, 93, 94, 95, 96, 97, 98, or 99% sequence identity to SEQ ID NO:16-22. In some embodiments, the peptide comprises an amino acid sequence selected from SEQ ID NO:23-29. In some embodiments, the peptide comprises an amino acid sequence with at least 80, 85, 86, 87, 88, 89, 90, 91, 92, 93, 94, 95, 96, 97, 98, or 99% sequence identity to SEQ ID NO:23-29. In some embodiments, the peptide comprises at least 10 amino acids. In some embodiments, the peptide comprises at least 6 contiguous amino acids of one of SEQ ID NO:7-29. In some embodiments, the peptide comprises at least 6, 7, 8, 9, 10, 11, 12, 13, 14, 15, 16, 17, or 18 (or any derivable range therein) contiguous amino acids of SEQ ID NOS:1-29. In some embodiments, the peptide consists of 10 amino acids. In some embodiments, the peptide consists of 8, 9, 10, 11, 12, 13, or 14 amino acids. In some embodiments, the peptide is less than 20 amino acids in length. In some embodiments, the peptide is less than 30, 29, 28, 27, 26, 25, 24, 23, 22, 21, 20, 19, 18, 17, 16, 15, 14, 13, 12, 11, 10, 9, 8, 7, or 6 amino acids (or any derivable range therein) in length. In some embodiments, the peptide is modified. In some embodiments, the modification comprises conjugation to a molecule. In some embodiments, the molecule comprises an antibody, a lipid, an adjuvant, or a detection moiety.

In some embodiments, the compositions of the disclosure are formulated as a vaccine. In some embodiments, the compositions and methods of the disclosure provide for prophylactic therapies to prevent brain cancer. In some embodiments, the compositions and methods of the disclosure provide for therapeutic therapies to treat existing cancers, such as for the treatment of patients with a brain tumor. In some embodiments, the composition further comprises an adjuvant. Adjuvants are known in the art and include, for example, TLR agonists and aluminum salts. Other adjuvants include IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. Further embodiments of adjuvants include amorphous aluminum hydroxyphosphate sulfate (AAHS), aluminum hydroxide, aluminum phosphate, potassium aluminum sulfate, the combination of monophosphoryl lipid A (MPL) and aluminum salt, oil in water emulsion composed of squalene, a liposomal formulation of MPL and QS-21 (a natural compound extracted from the Chilean soapbark tree), and cytosine phosphoguanine (CpG), a synthetic form of DNA that mimics bacterial and viral genetic material.

In some embodiments, the dendritic cell comprises a mature dendritic cell. In some embodiments, the cell is a cell with an HLA type selected from HLA-A, HLA-B, or HLA-C. In some embodiments, the cell is a cell with an HLA type selected from HLA-A*02:01, HLA-A*03 :01, HLA-A*23 :01, HLA-A*68:02, HLA-B*07:05, HLA-B*18:01, HLA-B*40:01, HLA-C*03:03, HLA-C*14:02, or HLA-C*15:02.

In some embodiments the methods of the disclosure further comprise screening the dendritic cell for one or more cellular properties. In some embodiments, the method further comprises contacting the cell with one or more cytokines or growth factors. In some embodiments, the one or more cytokines or growth factors comprises GM-CSF. In some embodiments, the cellular property comprises cell surface expression of one or more of CD86, HLA, and CD14. In some embodiments, the dendritic cell is derived from a CD34+hematopoietic stem or progenitor cell.

In some embodiments, the dendritic cell is derived from a peripheral blood monocyte (PBMC). In some embodiments, the dendritic cells is isolated from PBMCs. In some embodiments, the dendritic cells are cells in which the DCs are derived from are isolated by leukaphereses.

In some embodiments, the composition further comprises one or more cytokines, growth factors, or adjuvants. In some embodiments, the composition comprises GM-CSF. In some embodiments, the peptide and GM-CSF are linked. In some embodiments, the composition is determined to be serum-free, mycoplasma-free, endotoxin-free, and sterile. In some embodiments, the peptide is on the surface of the dendritic cell. In some embodiments, the peptide is bound to a MHC molecule on the surface of the dendritic cell. In some embodiments, the composition is enriched for dendritic cells expressing CD86 on the surface of the cell. In some embodiments, the dendritic cell is derived from a CD34+hematopoietic stem or progenitor cell. In some embodiments, the dendritic cell is derived from a peripheral blood monocyte (PBMC). In some embodiments, the dendritic cells or cells in which the DCs are derived are isolated by leukaphereses.

In some embodiments of the disclosure, the cell comprises a stem cell, a progenitor cell, or a T cell. In some embodiments, the cell comprises a hematopoietic stem or progenitor cell, a T cell, or an induced pluripotent stem cell (iPSC).

In some embodiments, the method comprises administering a cell or a composition comprising a cell and wherein the cell comprises an autologous cell. In some embodiments, the cell comprises a non-autologous cell.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of any of” the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-C provides a process to generate antigenic peptides utilizing RNA-seq data derived from neoplastic tissue in accordance with an embodiment.

FIG. 2 provides a process to generate antigenic peptides utilizing RNA-seq data and mass spectrometry data derived from neoplastic tissue in accordance with an embodiment.

FIG. 3 provides an example of process Isoform peptides from RNA splicing for Immunotherapy target Screening (IRIS) that can be used to identify peptides for T cell receptor and chimeric antigen receptor therapies in accordance with an embodiment. Shown is the Workflow for IRIS, integrating computational modules, large-scale reference RNA-Seq panels, and dedicated statistical testing programs. IRIS has three main modules: RNA-Seq data processing (top), in silico screening (middle), and TCR/CAR-T target prediction (bottom). The prediction module includes an option for proteo-transcriptomics integration of RNA-Seq and MS data.

FIG. 4. IRIS: A big data-powered platform for discovering AS-derived cancer immunotherapy targets. Stepwise results of IRIS to identify AS-derived cancer immunotherapy targets from 22 GBM samples (top). Identified skipped-exon (SE) events from the IRIS data-processing module were screened against tissue-matched normal panel (Normal Brain') to identify tumor-associated events (‘Primary’ set), followed by tumor panel and normal panel to identify tumor-recurrent and tumor-specific events, respectively (‘Prioritized’ set). After constructing splice junction peptides of tumor isoforms, TCR/CAR-T targets were predicted. As an illustrative example, IRIS readouts for prioritized candidate TCR targets are shown (bottom). Violin plots (left) show PSI values of individual AS events across GBM (GBM-input') versus three reference panels. Dots (middle) summarize screening results. Darker-colored dots indicate stronger tumor features (association/recurrence/specificity) versus each reference panel. FC is estimated fold change of tumor isoform' s proportion in GBM versus tissue-matched normal panel (‘Brain’). Predicted HLA-epitope binding (right) is output of prediction module. Preferred features for immunotherapy targets in this study are shown in blue. Amino acids at splice junctions in epitopes are underlined. ‘Best HLA’ is HLA type with best predicted affinity (median IC50) for given splice-junction epitope. ‘#Pt. w/HLA’ is number of patients with HLA type(s) predicted to bind to a given epitope. Three epitopes in TME62 and PLA2G6 (blue) were predicted to bind to common HLA types (HLA-A02:01 and HLA-A03:01) and were selected for experimental validation. Figure discloses SEQ ID NOS 7, 9, 10, 12, 11, 13, 15, 21, 22, 27, and 29, respectively, in order of appearance.

FIG. 5A-C. IRIS-predicted AS-derived TCR targets recognized by CD3⁺CD8⁺ T cells in tumors and peripheral blood from patients. a, Summary of dextramer-based validation of IRIS-predicted AS-derived epitopes. PBMCs and/or TILs from four HLA-A03 and two HLA-A02 patients were tested for recognition of IRIS-predicted epitopes. Within each HLA type, epitopes are listed by order of tumor specificity (high to low) versus normal panel (11 normal nonbrain tissues). Reactivity (‘Positive’, ‘Marginal’, or ‘Negative’) in assay was evaluated as percentage of dextramer-labeled cells among PBMCs/TILs (>0.1%, 0.01%-0.1%, or <0.01% of CD3⁺CD8⁺cells, respectively) after subtracting negative control (nonhuman peptide). ‘Dextramer assay summary’ was determined by the mean percent reactivity of CD3⁺CD8⁺cells across individual tests. b, Flow cytometric analysis showing that ex vivo-expanded TILs from one HLA-A03 patient (LB2867) contained T cells that recognized epitope KIGRLVTRK (SEQ ID NO:29). Rows correspond to cells that recognize APC- and PE-labeled dextramers (top), only PE-labeled dextramers (middle), or only APC-labeled dextramers (bottom). Percentages of epitope-specific cells are shown. c, Immune profiling results revealing immune repertoire composition of KIGRLVTRK (SEQ ID NO:29)-specific T cells from one patient (LB2867). The scRNA-Seq assay was performed on sorted KIGRLVTRK (SEQ ID NO:29)-specific T cells, whereas pairSEQ and immunoSEQ assays captured TCR clones from bulk TIL RNAs of same patient. Table (left) lists seven most abundant T-cell clones from scRNA-Seq, with percentages of matching CDR3 sequences from TCR (3 chains. *For pairSEQ and immunoSEQ, percentages are the best frequencies of matching TCR pair or (3-chain clones. The 3D scatterplot (right) shows that these approaches converged on three dominant TCR clones. For comparison, the same epitope in the table and 3D scatterplot are identified by use of the same color for the sequence (table) and text box (plot). Figure discloses SEQ ID NOS 29, 557, 22, 556, 27, 227, 62, 30-43, 33, 31, and 35, respectively, in order of appearance.

FIG. 6A-C. RNA-Seq big-data reference panels in IRIS. a, Exon-based principal component analysis (PCA) of RNA-Seq data of 9,662 samples from 53 normal tissues from the GTEx consortium. Samples from the same histological site are grouped by color. Samples from different subregions of the same histological site are differentiated by different shapes. b, Summary of 53 normal tissues from the GTEx consortium. Data for all 53 tissues are available to IRIS users as a reference panel of normal tissues. In the present study, 11 selected vital tissues (heart, skin, blood, lung, liver, nerve, muscle, spleen, thyroid, kidney, and stomach) were used for the ‘normal panel’. ‘Events Selected’ represent AS events with an average count ≥10 reads for the sum of all splice junctions across all samples in that tissue. c, Summary of the tumor reference panel (TCGA tumor samples relevant to GBM). ‘Events Selected’ represent AS events with an average count ≥10 reads for the sum of all splice junctions across all samples in that tumor type.

FIG. 7A-B. Identification of AS events that are prone to measurement errors due to technical variances across big-data reference panels. a, Computational workflow to create a ‘blacklist’ of error-prone AS events. Normal 76-bp RNA-Seq reads were artificially trimmed to 48 bp. RNA-Seq files (76- and 48-bp) were aligned by using two different aligners (Tophat and STAR). AS events were quantified by rMATS-turbo. AS events with statistically significant differences in PSI values among RNA-Seq datasets with distinct technical conditions were identified and included in a blacklist. b, Scatter plots comparing PSI values of GTEx normal brain RNA-Seq data estimated under distinct technical conditions (read lengths: 48- and 76-bp, aligners: STAR and Tophat). ‘Significantly different’ AS events were defined as those with significantly different PSI values (p<0.05, abs(Δψ)>0.05 from paired t-test).

FIG. 8A-B. CAR-T target prediction by IRIS. a, Computational workflow to annotate protein extracellular domain (ECD)-associated AS events for CAR-T target discovery. b, Five examples of IRIS-identified AS-derived CAR-T targets for 22 GBM samples. Position of the ECD in amino acid (aa) sequence was obtained from UniProtKB.

FIG. 9A-E. Proteo-transcriptomic analysis of HLA presentation of AS-derived epitopes in normal and tumor cell lines. a, Proteo-transcriptomics workflow adopted by IRIS to discover splice-junction peptides in MS datasets. IRIS inputs MS data (right), such as whole-cell proteomics, surfaceomics, or immunopeptidomics (HLA peptidomics) data. RNA-Seq-based custom proteome library is constructed and searched using MSGF+. b, Summary of HLA presentation of AS-derived epitopes in JeKo-1 (lymphoma) and B-LCL (normal) cell lines. Peptide-spectrum matches ('PSMs') and ‘Unique peptides’ are provided by MSGF+ with a target-decoy FDR of 5%. ‘Predicted AS epitopes’ are generated by the IRIS prediction module, which utilizes IEDB predictors. AS epitopes that are predicted by IRIS and detected in the MS data are considered ‘MS-validated AS epitopes’. c, Percentage of IRIS-predicted AS-derived epitopes among all MS-detected peptides. Graph shows the percentage of all MS-detected peptides that are IRIS-predicted AS-derived epitopes (y-axis) as a function of the MSGF+target-decoy FDR (x-axis). d, Preferential detection of high-affinity AS-derived peptides in MS data. Graph shows the number of AS-derived peptides detected in JeKo-1 MS data (y-axis) as a function of the MSGF+target-decoy FDR (x-axis). Peptides with high (IC₅₀<500 nM; Pred+, orange) and low (IC₅₀>500 nM; Pred-, grey) predicted HLA binding affinities are shown. e, Heatmap depiction of distribution of AS-derived epitopes in JeKo-1 MS immunopeptidome, as a function of predicted HLA binding affinity and transcript expression level. AS-derived peptides are binned by the corresponding transcripts' expression levels and IEDB-predicted binding affinity scores. Heatmap is colored from red (high) to yellow (90^{th} percentile) to blue (low), reflecting the proportion of IRIS-predicted AS-derived epitopes that are MS-detected in each bin.

FIG. 10A-D. Consistent distributions of high-frequency TCR clones in one patient's TIL population revealed by multiple TCR sequencing approaches. a, Scatter plot comparing scRNA-Seq and bulk TIL pairSEQ for detection of high-frequency TCR clones. Graph shows frequency detected from bulk TIL samples using pairSEQ (y-axis) and scRNA-Seq on dextramer-positive sorted TIL samples (x-axis). As a complementary validation of scRNA-Seq, clonotypes from pairSEQ were matched to scRNA-Seq results by either CDR3 pairs or β chains, whichever matched best. The 10 most abundant TCR clones by scRNA-Seq that overlapped with clones detected by bulk TIL pairSEQ are circled. b, Table showing CDR3 amino acid sequences of the 10 most abundant TCR clones detected by scRNA-Seq and their corresponding detection frequencies by bulk TIL pairSEQ. As a complementary validation of scRNA-Seq, clonotypes from pairSEQ were matched to scRNA-Seq results by either CDR3 pairs or β chains, whichever matched best. c, Scatter plot comparing bulk TIL immunoSEQ and bulk TIL pairSEQ for detection of high-frequency TCR clones. Graph shows frequency detected from bulk TIL samples using immunoSEQ (y-axis) and pairSEQ (x-axis). Clonotypes from immunoSEQ were matched to pairSEQ results by the best CDR3 β chains. Four high-frequency overlapping clones from both methods are circled and color-coded, with (3-chain CDR3 amino acid sequences and frequencies by each method shown in boxes. d, Scatter plot comparing scRNA-Seq and bulk TIL immunoSEQ for detection of high-frequency TCR clones. Graph shows frequency detected from bulk TIL samples using immunoSEQ (y-axis) and scRNA-Seq on dextramer-positive sorted TIL samples (x-axis). As a complementary validation of scRNA-Seq, clonotypes from immunoSEQ were matched to scRNA-Seq results by the best CDR3 β chains. Three high-frequency overlapping clones from both methods are circled and color-coded, with β-chain CDR3 amino acid sequences and frequencies by each method shown in boxes. Figure discloses SEQ ID NOS 30, 32, 34, 36, 38, 40, 42, 714, 715, 581, 746, 31, 33, 35, 37, 39, 41, 43, 572, 574, and 580 (in order of columns) and 558, 31, 33, 39, 33, 35, and 31, respectively, in order of appearance.

FIG. 11: IRIS: A big data-powered platform for discovering AS-derived cancer immunotherapy targets. Stepwise results of IRIS to identify AS-derived cancer immunotherapy targets from 22 GBM samples (top). Identified skipped-exon (SE) events from the IRIS data-processing module were screened against tissue-matched normal panel (Normal Brain') to identify tumor-associated events (‘Primary’ set), followed by tumor panel and normal panel to identify tumor-recurrent and tumor-specific events, respectively (‘Prioritized’ set). After constructing splice junction peptides of tumor isoforms, TCR/CAR-T targets were predicted. As an illustrative example, IRIS readouts for prioritized candidate TCR targets are shown (bottom). Violin plots (left) show PSI values of individual AS events across GBM (GBM-input') versus three reference panels. Dots (middle) summarize screening results. Darker-colored dots indicate stronger tumor features (association/recurrence/specificity) versus each reference panel. FC is estimated fold change of tumor isoform's proportion in GBM versus tissue-matched normal panel (‘Brain’). Predicted HLA-epitope binding (right) is output of prediction module. Preferred features for immunotherapy targets in this study are shown in blue. Amino acids at splice junctions in epitopes are underlined. ‘Best HLA’ is HLA type with best predicted affinity (median IC₅₀) for given splice-junction epitope. ‘#Pt. w/HLA’ is number of patients with HLA type(s) predicted to bind to a given epitope. Figure discloses SEQ ID NOS:1371, 1396, 1397, 1380, 1398, 1399, 1400, 1401, 1402, 21, and 22, respectively, in order of appearance.

### DETAILED DESCRIPTION OF THE INVENTION

Aberrant alternative splicing (AS) is widespread in cancer, leading to an extensive but largely unexploited repertoire of potential immunotherapy targets. This disclosure describes computational platforms leveraging large-scale cancer and normal transcriptomics data to discover AS-derived tumor antigens for T-cell receptor (TCR) and chimeric antigen receptor T-cell (CAR-T) therapies. Applying AS identifying computational platforms to RNA-Seq data from 22 glioblastomas resected from patients, the inventors identified candidate epitopes and validated their recognition by patient T cells, demonstrating platforms' utility for expanding targeted cancer immunotherapy.

1. Identification and Synthesis of Neoplastic Tissue Antigens

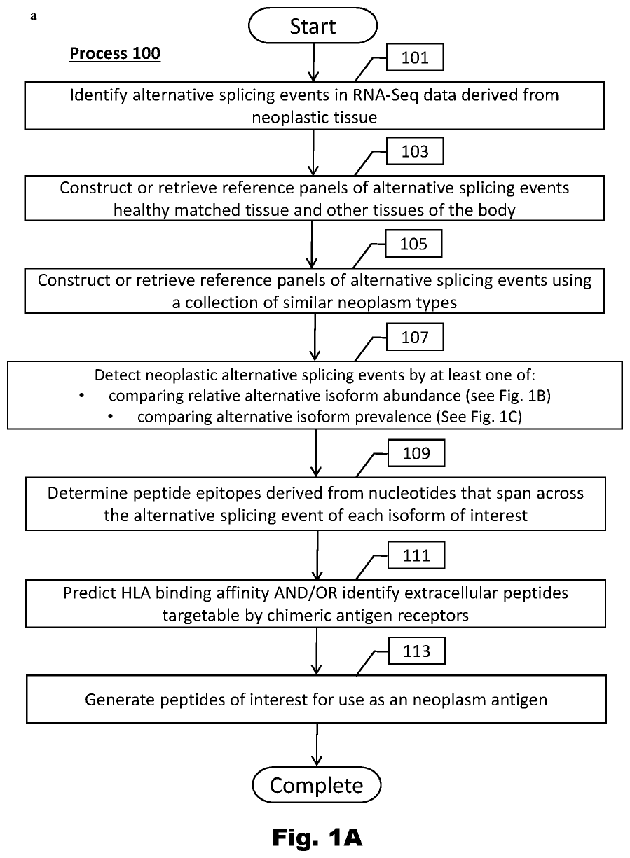

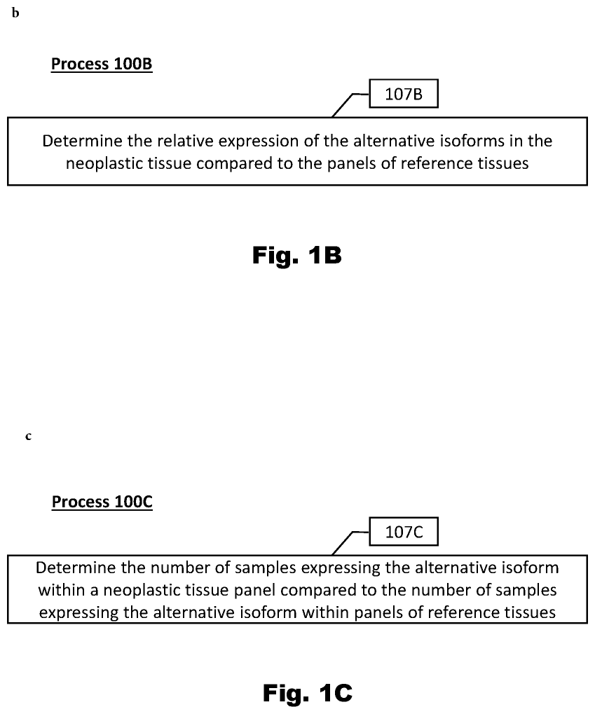

An embodiment of a process to identify and synthesize neoplastic tissue antigens is illustrated in FIG. 1A. This embodiment is directed to utilizing RNA-seq data derived from neoplastic tissue to identify AS events, especially in neoplastic tissue, which in turn is utilized to identify antigens derived from the AS events. Various comparative and statistical methods are utilized to rank AS events and the antigens.

Process 100 can begin with identifying (101) AS event in RNA seq data derived from neoplastic tissue. AS events include (but are not limited to) exon skipping, an alternative 3′ splice site, an alternative 5′ splice site, and intron retention. For AS events identification applications described within, RNA sequencing provides a facile method to obtain sequence data, as it is typically abundant in the biological source, can be easily sequenced by known methods, readily available in numerous public and private databases, has intronic sequences already removed, and many exon reference databases exist for post-sequencing data analysis.

The source of RNA sequence data can be derived de novo (i.e., from biological tissue), or from a public or private database. Several methods can be utilized to derive RNA sequence data from biological tissue (or a collection of biological tissues). Generally, RNA molecules are extracted from tissue, prepped to be sequenced, and then run on a sequencer. For example, RNA can be extracted from a human tissue source, then prepped into a sequence library, and sequenced on a next-generation sequencing platform, such as those manufactured by Illumina, Inc. (San Diego, Calif.). Neoplastic tissue sources include (but are not limited to) tumor biopsy, nodal biopsy, surgical resection, and liquid/soft biopsies. Liquid and soft biopsies can be used to collect circulating neoplastic cells or cell-free nucleic acids, and include (but not limited to) blood, plasma, lymph, cerebral spinal fluid, urine, and stool. In many embodiments, biopsies are extracted from patients having been diagnosed with a particular neoplasm.

In some embodiments, RNA sequence data can be derived from an available database. For example, transcriptome data can be obtained from the National Center for Biotechnology Information (NCBI), Reference Sequence Database (RefSeq), Genotype-Tissue Expression Portal (GTEx), and The Cancer Genome Atlas Program (TCGA) databases. Sequence data could be in any appropriate sequence read format, including (but not limited to) single or paired-end reads.

Any appropriate neoplastic tissue can be analyzed, including (but not limited to) acute lymphoblastic leukemia (ALL), acute myeloid leukemia (AML), anal cancer, astrocytomas, basal cell carcinoma, bile duct cancer, bladder cancer, breast cancer, Burkitt's lymphoma, cervical cancer, chronic lymphocytic leukemia (CLL) chronic myelogenous leukemia (CIVIL), chronic myeloproliferative neoplasms, colorectal cancer, diffuse large B-cell lymphoma, endometrial cancer, ependymoma, esophageal cancer, esthesioneuroblastoma, Ewing sarcoma, fallopian tube cancer, follicular lymphoma, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T cell lymphoma, testicular cancer, thymoma, thyroid cancer, uterine cancer, vaginal cancer, and vascular tumors.

In many embodiments, RNA is processed before analysis. Any appropriate method can be used to process sequence data. For example, the sequence data can be trimmed with the publicly available TrimGalore (http://www.bioinformatics.babraham.ac.uk/projects/trim_galore!) or cutAdapt (https://cutadapt.readthedocs.io/en/stable/) methods, which remove adapter sequences and trim poor-quality bases. Mapping can be performed with any appropriate annotated genome, such as, for example, UC SC's hg19 (http ://supportillumina.com/sequencing/sequencing_software/igenome.html) and alignment tool, such as, for example, B owtie2 (http://bowtie-bio.sourceforge.net/bowtie2/index.shtml), TopHat (https://ccb.jhu.edu/software/tophat/index.shtml), and STAR (https://github.com/alexdobin/STAR). Genes and their exons can be identified and their relative expression level determined. For instance, quantification of gene expression and AS events can be determined by GENCODE package (Harrow, J. et al. Genome Res. 22, 1760-1774 (2012), the disclosure of which is incorporated herein by reference). Potential false-positive events can be removed by using a blacklist of AS events whose quantification across diverse RNA-Seq datasets is error-prone due to technical variances such as read length. Based on expression levels of exons, in several embodiments, splice-junction counts are determined by an appropriate method, such as the rMATS package (S. Shen, et al. Proc. Natl. Acad. Sci. 111, E5593—E5601 (2014), the disclosure of which is incorporated herein by reference). Splice-junction counts can be utilized to find putative skipped exons, included exons, alternative 3′ splice sites, alternative 5′ splice sites, and/or retained introns in the sequencing result. In some embodiments, measurements of AS events, including splice junction count and percent-spliced-in (PSI) metric, are computed. Processing of the data will be dependent on the users' goal, and thus adaptable to the results desired. Although only a few methods of trimming, processing, and mapping sequence data are disclosed, it should be understood many more methods exist and are covered by various embodiments of the invention.

Returning back to FIG. 1, reference panels of AS events in healthy matched tissue and other tissues of the body are constructed or retrieved (103). In many embodiments, healthy matched tissue is the same tissue origin as the neoplastic tissue, but has not transformed into a neoplasm. For instance, in some embodiments, healthy matched tissue of glioblastoma (GBM) is brain tissue. In some embodiments, other tissues of the body include any tissue that is not the source of the neoplasm. Tissues of single individual or tissue of collections of individuals can be analyzed. Analysis can be done on RNA-seq data derived from a single individual or a collection of individuals. For each non-neoplastic tissue to be analyzed, the RNA-seq data can be utilized to determine expression levels of exons, which can be utilized to determine splice-junction counts and putative skipped and/or included exons. These data can be stored to be utilized for comparisons with the neoplastic tissue of interest to be analyzed.

In a number of embodiments, utilizing the panel of AS events in healthy match tissue and the AS events of the neoplastic tissue, the relative abundance of AS events can be computed. The PSI is the percent of a particular isoform included in an AS event in the neoplastic tissue or the healthy matched or other heathy tissue and can be utilized for any type of AS event, including (but not limited to) exon skipping, an alternative 3′ spice site, an alternative 5′ spice site, and intron retention. Generally, a high PSI value in the neoplastic tissue, as compared to healthy match tissue, indicates including of the genetic material and a low PSI value in the neoplastic tissue indicates the neoplastic tissue spices out the genetic material. For example, in regards to exon-skipping, neoplastic tissue isoforms can be either an exon-skipped isoform (low PSI) or an exon-included isoform (high PSI), as compared to the tissue-matched normal panel.

In addition, a reference panel of AS events of a collection of similar neoplasm types is constructed or retrieved (105). Similar neoplasm types can be neoplasms having the same tissue origin. For instance, to construct a reference panel for GBM, other brain tumor sequencing data can be utilized, including (but not limited to) other samples of GBM and/or lower-grade glioma. The RNA-seq data of the collection of samples can be utilized to determine splice-junction counts and putative skipped exons, included exons, alternative 3′ splice sites, alternative 5′ splice sites, and/or retained introns. These data can be stored to be utilized for comparisons with the neoplastic tissue of interest to be analyzed.

Process 100 also detects (107) putative recurrent AS event candidates. In some embodiments, recurrent AS event candidates are determined comparing relative abundance of alternative isoforms (FIG. 1B). In some embodiments, putative recurrent AS event candidates are determined by comparing prevalence of alternative isoforms (FIG. 1C). In some embodiments, recurrent AS event candidates are determined comparing relative abundance and comparing prevalence of alternative isoforms.

As depicted in FIG. 1B, process 100B determines (107B) the relative abundance of the alternative isoforms by determining the relative expression of the alternative isoforms in the neoplastic tissue, as compared to the relative expression of the alternative isoforms in the panels of reference tissues (e.g., healthy matched tissue, other tissues, and similar neoplasm types). In many embodiments, statistical differential testing is utilized to determine the significance of a putative AS event candidate, as determined by the relative expression of an AS event. In some embodiments, a significant AS event is one that is a significant as determined by the resulting p-value of a statistical test comparing neoplastic tissue and a reference tissue. Statistical tests include (but are not limited to) parametric tests (e.g. two-sided/one sided t-test) and non-parametric tests (e.g. Mann-Whitney U test). In some embodiments, the difference of PSI values between comparing neoplastic tissue and reference tissue is utilized to identify significant AS events. In particular embodiments, a neoplastic AS event is significant when it satisfies the following: 1) a significant p-value from a statistical test (e.g., p<0.01), and 2) a threshold of PSI value difference (e.g., abs(ΔΨN)>0.05).

In some embodiments, significance testing (e.g., t-tests) and equivalence testing (e.g., two one-sided t-tests (TOSTs)) is used to identify neoplasm-associated, neoplasm-recurrent, and neoplasm-specific AS events in group comparisons. Specifically, AS events can be compared with a reference tissue (e.g., healthy matched tissue) to identify neoplastic tissue-associated AS events, with other tissue types to determine neoplastic tissue-specificity of AS events, and with similar neoplasm types to evaluate recurrence of AS events. In some embodiments, an AS event is considered significantly different when it meets two requirements: (1) a significant p-value from the statistical test (defaults: p<0.01 for significance testing; p<0.05 for equivalence testing), and (2) a threshold of PSI value difference (default: abs(ΔΨ)>0.05 for significance testing; abs(ΔΨ)<0.05 for equivalence testing).

In several embodiments, an AS event is defined as neoplasm-recurrent by comparing a panel of neoplastic tissue data with a panel of reference tissue (e.g., healthy matched tissue). For instance, in some embodiments, a neoplasm-recurrent AS event is identified when 1) a significant p-value from the statistical test in the same direction as the corresponding neoplasm-associated AS event (e.g., p<0.01/number of neoplasm-associated events;), and 2) a threshold of PSI value difference (default: abs(ΔΨ)>0.05). In some embodiments, a Bonferroni correction is applied wen determining p-value from the statistical test, which may be helpful due to large sample sizes in reference panels.

Additionally, in some embodiments, a threshold of the number of significant comparisons against groups in the normal or neoplasm reference panel is used to determine whether AS-derived antigens are neoplasm-specific or neoplasm-recurrent. In various embodiments, the neoplasm panel data and/or reference panel data includes multiple individual groups (e.g., tissue types) and a threshold of the number of significant comparisons against groups in the normal or tumor reference panel is used to determine whether AS-derived antigens are tumor-specific or tumor-recurrent. For each AS event, various embodiments utilize a definition that the ‘neoplasm isoform’ is the isoform that is more abundant in neoplastic tissue than in the tissue-matched normal panel. Optionally, in some embodiments, to rank or filter targets, the ‘fold-change (FC) of neoplasm isoform’ is estimated as the FC of the neoplasm isoform's proportion in neoplasms compared to the tissue-matched normal panel. Furthermore, in some embodiments, targets are screened for a specific patient sample through a ‘personalized mode’. A personalized mode uses an outlier detection approach, combining a modified Tukey's rule and a threshold of PSI value difference of >5%.

As depicted in FIG. 1C, process 100C determines (107C) the prevalence of the alternative isoforms by determining the number of samples expressing the alternative isoform within a neoplastic tissue panel, as compared to the number of samples expressing the alternative isoform within the panels of reference tissues (e.g., healthy matched tissue, other tissues, and similar neoplasm types). In some of embodiments, a sample is considered to express a particular alternative isoform if the number of uniquely mapped junction read counts from RNA-seq data is greater than or equal to a junction count threshold.

Prevalence screening refers to the comparison the prevalence of a splice junction in a panel of neoplasm samples to one or more reference tissue samples. Specifically, in some embodiments, neoplasm samples of interest or related neoplasm samples, which can be selected from a neoplasm reference panel or other resource, are compared to reference tissue samples (e.g., tissue--matched normal samples or other normal tissue samples). In some embodiments, statistical tests (e.g., Fisher's exact test or chi-squared test) are employed to evaluate the difference of splice junction prevalence between the two groups in comparison. As a result, this allows identification of splice junctions prevalently expressed in neoplasm samples that are less observed in reference tissues. In some embodiments, to avoid false positive results by solely using read counts from RNA-seq, the same junction count information is used to calculate PSI-values and perform a relative abundance (PSI) based screening in parallel (see FIG. 1B). Employment of prevalence based methods has some advantages. For example, for annotated and unannotated splice junctions, this approach offers additional knowledge for prioritization. Furthermore, this approach detects unannotated splice junctions derived from novel splice sites without the need to rebuild the splice graph. This allows for the evaluation of both junction prevalence and relative abundance for confident detection of neoplasm-specific splicing events.

Returning back to FIG. 1A,peptide epitopes derived from nucleotides that span across the AS event of each isoform of interest are determined (109). In a number of embodiments, to obtain protein sequences of AS-derived neoplasm isoforms, peptide sequences are generated by translating splice-junction sequences into amino-acid sequences. In some embodiments, splice-junction sequences are translated into amino-acid sequences using known ORFs from the UniProtKB database (www.uniprot.org). In some embodiments, splice-junction sequences are translated into amino-acid sequences for each potential open reading frame (i.e., the three open reading frames dependent on triple nucleotide codon window), which is useful for isoform junction derived from alternative and/or novel splice sites. Within each AS event, the splice-junction peptide sequence for the neoplasm isoform can be compared to that of the alternative normal isoform, to ensure that the neoplasm isoform splice junction produces a distinct peptide. It is noted that a single splice junction can give rise to multiple putative epitopes with distinct peptide sequences

Process 100 also predicts (111) HLA binding affinity and/or identifies targetable extracellular peptides by TCR and/or chimeric antigen receptors. For TCR target prediction, a computational package can be employed which uses RNA-Seq data to characterize HLA class I alleles for each tumor sample to identify putative epitopes. In some, embodiments, the seq2HLA is used for TCR epitope identification (Boegel, S. et al. Genome Med. 4, 102 (2012), the disclosure of which is incorporated herein by reference). In addition, a computational package can predict the HLA binding affinities of candidate epitopes (e.g., the IEDB API from Vita, R. et al. Nucleic Acids Res. 43, D405—D412 (2015), the disclosure of which is herein incorporated by reference). The IEDB ‘recommended’ mode runs several prediction tools to generate multiple predictions of binding affinity, which can be summarized by a median IC₅₀ value. In some embodiments, a threshold of median(IC₅₀)<500 nM denotes a positive prediction for an AS-derived TCR target, but any appropriate binding affinity can be utilized.

For CAR-T cell target prediction, AS-derived tumor isoforms can be mapped to known protein extracellular domains (ECDs) to identify potential candidates for CAR-T cell therapy. Protein cellular localization information can be retrieved from the UniProtKB database (www.uniprot.org). To retrieve ECD information from the UniProtKB database, a search for the term ‘extracellular’ in topological annotation fields can be performed, including ‘TOP_DOM’, ‘TRANSMEM’, and ‘REGION’, in the flat file. In addition, BLAST (https://blast.ncbi.nlm.nih.gov/) can be used to map individual exons in the gene annotation to proteins with topological annotations. Furthermore, the BLAST result can be parsed to create annotations of the mapping between exons and ECDs in proteins. These pre-computed annotations can be queried to search for AS-derived peptides that can be mapped to protein ECDs as potential CAR-T cell targets.

Based on the results of HLA epitopes and CAR-T cell targets, peptides of interest can be generated (113) for use as a neoplasm antigen. Peptides can be synthesized directly (e.g., solid phase synthesis) or via molecular expression utilizing an expression vector and a host production cell.

While specific examples of identifying and synthesizing neoplastic tissue antigens are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for identifying and synthesizing neoplastic tissue antigens appropriate to the requirements of a given application can be utilized in accordance with various embodiments of the invention.

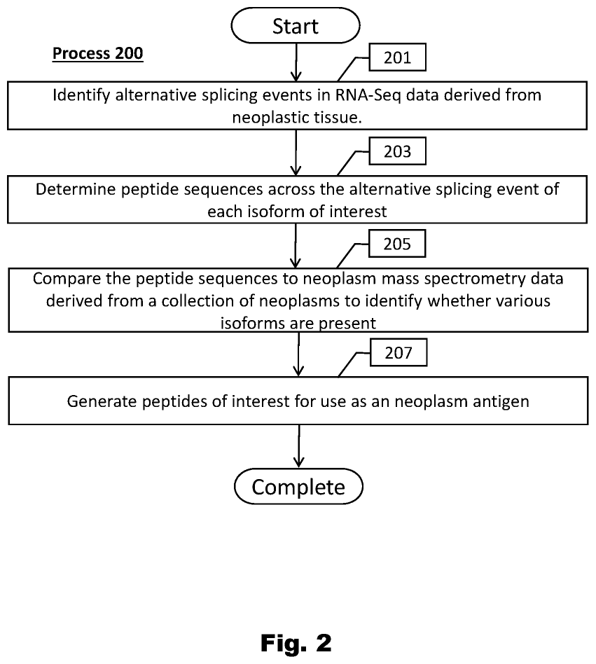

Provided in FIG. 2 is a process to identify and synthesize neoplastic tissue antigenic peptides integrating results of mass spectrometry data derived from a neoplastic tissue source. Process 200 can begin by identifying (201) alternative splicing events in RNA-Seq data derived from neoplastic tissue. In a manner similar to Process 100, RNA sequence data can be derived from a biological source or a database. In addition, RNA can be processed before analysis. Any appropriate method can be used to process sequence data as described herein. Potential false-positive events can be removed by using a blacklist of AS events whose quantification across diverse RNA-Seq datasets is error-prone due to technical variances such as read length. Based on expression levels of exons, in several embodiments, splice-junction counts are determined by an appropriate method, such as the rMATS package. Splice-junction counts can be utilized to find putative skipped exons, included exons, alternative 3′ splice sites, alternative 5′ splice sites, and/or retained introns in the sequencing result.

Peptide epitopes derived from nucleotides that span across the alternative splicing event of each isoform of interest is determined (203). In a number of embodiments, expression of the alternative isoforms in the neoplastic tissue compared to the panels of healthy matched tissue, other tissues, and similar neoplasm types. Generally, AS events can be compared with refrence tissue (e.g., healthy matched tissue) to identify neoplastic tissue-associated AS events, with other tissue types to determine neoplastic tissue-specificity of AS events, and with similar neoplasm types to evaluate recurrence of AS events. In some embodiments, putative splice junction candidates are determined comparing relative abundance. In some embodiments, putative splice junction candidates are determined by comparing prevalence. In some embodiments, putative splice junction candidates are determined comparing relative abundance and comparing prevalence.

Process 200 also compares (205) the peptide sequences to mass spectrometry data derived from a collection of neoplasms to identify whether various isoforms are present. In some embodiments, proteo-transcriptomic data is integrated by incorporating various types of MS data, such as whole-cell proteomics, surfaceome, or immunopeptidomics data, to validate RNA-Seq based target discovery at the protein level. Specifically, sequences of AS-derived peptides are mapped to canonical and isoform sequences of the reference human proteome (downloaded from UniProtKB). For immunopeptidomics data, fragment MS spectra can be searched against the RNA-Seq based custom proteome library with no enzyme specificity. In some embodiments, the search length is limited to 7-15 amino acids. In some embodiments, the target-decoy approach is employed to control the false discovery rate (FDR) or ‘QValue’ at 5%.

Based on the results searching MS data for hits, peptides of interest can be generated (207) for use as a neoplasm antigen. Peptides can be synthesized directly (e.g., solid phase synthesis) or via biological translation utilizing an expression vector and a host production cell.

While specific examples of identifying and synthesizing neoplastic tissue antigens utilizing MS data are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for identifying and synthesizing neoplastic tissue antigens utilizing MS data appropriate to the requirements of a given application can be utilized in accordance with various embodiments of the invention.

### II. APPLICATIONS OF ANTIGENIC PEPTIDES

Various embodiments are directed to development of and use of antigenic peptides that have been identified from neoplastic tissue. In many embodiments, antigenic peptides are produced by chemical synthesis or by molecular expression in a host cell. Peptides can be purified and utilized in a variety of applications including (but not limited to) assays to determine peptide immunogenicity, assays to determine recognition by T cells, peptide vaccines for treatment of cancer, development of modified TCRs of T cells, development of antibodies, and development of CAR-T cells to recognize extracellular peptides.

Peptides can be synthesized chemically by a number of methods. One common method is to use solid-phase peptide synthesis (SPPS). Generally, SPPS is performed by repeating cycles of alternate N-terminal deprotection and coupling reactions, building peptides from the c-terminus to the n-terminus. The c-terminus of the first amino acid is coupled the resin, wherein then the amine is deprecated and then coupled with the free acid of the second amino acid. This cycle repeats until the peptide is synthesized.

Peptides can also be synthesized utilizing molecular tools and a host cell. Nucleic acid sequences corresponding with antigenic peptides can be synthesized. In some embodiments, synthetic nucleic acids synthesized in in vitro synthesizers (e.g., phosphoramidite synthesizer), bacterial recombination system, or other suitable methods. Furthermore, synthesized nucleic acids can be purified and lyophilized, or kept stored in a biological system (e.g., bacteria, yeast). For use in a biological system, synthetic nucleic acid molecules can be inserted into a plasmid vector, or similar. A plasmid vector can also be an expression vector, wherein a suitable promoter and a suitable 3′-polyA tail is combined with the transcript sequence.

Embodiments are also directed to expression vectors and expression systems that produce antigenic peptides or proteins. These expression systems can incorporate an expression vector to express transcripts and proteins in a suitable expression system. Typical expression systems include bacterial (e.g., E. coli), insect (e.g., SF9), yeast (e.g., S. cerevisiae), animal (e.g., CHO), or human (e.g., HEK 293) cell lines. RNA and/or protein molecules can be purified from these systems using standard biotechnology production procedures.

Assays to determine immunogenicity and/or TCR binding can be performed. One such as is the dextramer flow cytometery assay. Generally, custom-made HLA-matched MHC Class I dextramer:peptide (pMHC) complexes are developed or purchased (Immudex, Copenhagen, Denmark). T cells from peripheral blood mononuclear cells (PBMCs) or tumor-infiltrating lymphocytes (TILs) are incubated the pMHC complexes and stained, which are then run through a flow cytometer to determine if the peptide is capable of binding a TCR of a T cell.

### III. ENGINEERED T CELL RECEPTORS

T-cell receptors comprise two different polypeptide chains, termed the T-cell receptor α (TCRα) and β (TCRβ) chains, linked by a disulfide bond. These α:β heterodimers are very similar in structure to the Fab fragment of an immunoglobulin molecule, and they account for antigen recognition by most T cells. A minority of T cells bear an alternative, but structurally similar, receptor made up of a different pair of polypeptide chains designated γ and δ. Both types of T-cell receptor differ from the membrane-bound immunoglobulin that serves as the B-cell receptor: a T-cell receptor has only one antigen-binding site, whereas a B-cell receptor has two, and T-cell receptors are never secreted, whereas immunoglobulin can be secreted as antibody.

Both chains of the T-cell receptor have an amino-terminal variable (V) region with homology to an immunoglobulin V domain, a constant (C) region with homology to an immunoglobulin C domain, and a short hinge region containing a cysteine residue that forms the interchain disulfide bond. Each chain spans the lipid bilayer by a hydrophobic transmembrane domain, and ends in a short cytoplasmic tail.

The three-dimensional structure of the T-cell receptor has been determined. The structure is indeed similar to that of an antibody Fab fragment, as was suspected from earlier studies on the genes that encoded it. The T-cell receptor chains fold in much the same way as those of a Fab fragment, although the final structure appears a little shorter and wider. There are, however, some distinct differences between T-cell receptors and Fab fragments. The most striking difference is in the Cα domain, where the fold is unlike that of any other immunoglobulin-like domain. The half of the domain that is juxtaposed with the Cβ domain forms a β sheet similar to that found in other immunoglobulin-like domains, but the other half of the domain is formed of loosely packed strands and a short segment of α helix. The intramolecular disulfide bond, which in immunoglobulin-like domains normally joins two β strands, in a Cα domain joins a β strand to this segment of α helix.

There are also differences in the way in which the domains interact. The interface between the V and C domains of both T-cell receptor chains is more extensive than in antibodies, which may make the hinge joint between the domains less flexible. And the interaction between the Cα and Cβ domains is distinctive in being assisted by carbohydrate, with a sugar group from the Cα domain making a number of hydrogen bonds to the Cβ domain. Finally, a comparison of the variable binding sites shows that, although the complementarity-determining region (CDR) loops align fairly closely with those of antibody molecules, there is some displacement relative to those of the antibody molecule. This displacement is particularly marked in the Vα CDR2 loop, which is oriented at roughly right angles to the equivalent loop in antibody V domains, as a result of a shift in the β strand that anchors one end of the loop from one face of the domain to the other. A strand displacement also causes a change in the orientation of the Vβ CDR2 loop in two of the seven Vβ domains whose structures are known. As yet, the crystallographic structures of seven T-cell receptors have been solved to this level of resolution.

Embodiments of the disclosure relate to engineered T cell receptors. The term “engineered” refers to T cell receptors that have TCR variable regions grafted onto TCR constant regions to make a chimeric polypeptide that binds to peptides and antigens of the disclosure. In certain embodiments, the TCR comprises intervening sequences that are used for cloning, enhanced expression, detection, or for therapeutic control of the construct, but are not present in endogenous TCRs, such as multiple cloning sites, linker, hinge sequences, modified hinge sequences, modified transmembrane sequences, a detection polypeptide or molecule, or therapeutic controls that may allow for selection or screening of cells comprising the TCR.

In some embodiments, the TCR comprises non-TCR sequences. Accordingly, certain embodiments relate to TCRs with sequences that are not from a TCR gene. In some embodiments, the TCR is chimeric, in that it contains sequences normally found in a TCR gene, but contains sequences from at least two TCR genes that are not necessarily found together in nature.

In some embodiments the engineered TCRs of the disclosure comprise a variable as shown below:

| Description | Sequence |
|---|---|
| TCR1-alpha-variable; | MLLLLVPAFQVIFTLGGTRAQSVTQLDSQVPVFEEAPVE |
| TRAV8-6*01 (uppercase) | LRCNYSSSVSVYLFWYVQYPNQGLQLLLKYLSGSTLVE |
| TRAJ54*01 (lowercase) | SINGFEAEFNKSQTSFHLRKPSVHISDTAEYFCAVHeiqga |
| | qklvfgqgtrltinpn (SEQ ID NO: 44) |
| TCR1-alpha CDR3 | CAVHEIQGAQKLVF (SEQ ID NO: 30) |
| TCR1-beta-variable; | MGTSLLCWVVLGFLGTDHTGAGVSQSPRYKVTKRGQD |
| TRBV7-6*01 (uppercase) | VALRCDPISGHVSLYWYRQALGQGPEFLTYFNYEAQQ |
| TRBJ2-7*01 (lowercase) | DKSGLPNDRFSAERPEGSISTLTIQRTEQRDSAMYRCAS |
| | SFGVsyeqyfgpgtrltvt (SEQ ID NO: 45) |
| TCR1-beta CDR3 | CASSFGVSYEQYF (SEQ ID NO: 31) |
| TCR2-alpha-variable; | MSLSSLLKVVTASLWLGPGIAQKITQTQPGMFVQEKEA |
| TRAV14/DV4*01 | VTLDCTYDTSDPSYGLFWYKQPSSGEMIFLIYQGSYDQ |
| (uppercase) TRAJ4*01 | QNATEGRYSLNFQKARKSANLVISASQLGDSAMYFCA |
| (lowercase) | MRPLggynklifgagtrlavhp (SEQ ID NO: 46) |
| TCR2-alpha CDR3 | CAMRPLGGYNKLIF (SEQ ID NO: 32) |
| TCR2-beta-variable; | MGCRLLCCAVLCLLGAVPIDTEVTQTPKHLVMGMTNK |
| TRBV4-1*01 (uppercase) | KSLKCEQHMGHRAMYWYKQKAKKPPELMFVYSYEKL |
| TRBJ2-1*01 (lowercase) | SINESVPSRFSPECPNSSLLNLHLHALQPEDSALYLCASS |
| | QAAneqffgpgtrltvl (SEQ ID NO: 47) |
| TCR2-beta CDR3 | CASSQAANEQFF (SEQ ID NO: 33) |
| TCR3-alpha-variable; | MKTFAGFSFLFLWLQLDCMSRGEDVEQSLFLSVREGDS |
| TRAV5*01 (uppercase) | SVINCTYTDSSSTYLYWYKQEPGAGLQLLTYIFSNMDM |
| TRAJ20*01 (lowercase) | KQDQRLTVLLNKKDKHLSLRIADTQTGDSAIYFCAEEgd |
| | rdyklsfgagttvtvran (SEQ ID NO: 48) |
| TCR3-alpha CDR3 | CAEEGDRDYKLSF (SEQ ID NO: 34) |
| TCR3-beta-variable; | MGTRLLCWVVLGFLGTDHTGAGVSQSPRYKVAKRGQ |
| TRBV7-8*01 (uppercase) | DVALRCDPISGHVSLFWYQQALGQGPEFLTYFQNEAQL |
| TRBJ2-7*01 (lowercase) | DKSGLPSDRFFAERPEGSVSTLKIQRTQQEDSAVYLCAS |
| | TGRSGrseqyfgpgtrltvt (SEQ ID NO: 49) |
| TCR3-beta CDR3 | CASTGRSGRSEQYF (SEQ ID NO: 35) |
| TCR1-alpha-complete gene | MLLLLVPAFQVIFTLGGTRAQSVTQLDSQVPVFEEAPVE |
| | LRCNYSSSVSVYLFWYVQYPNQGLQLLLKYLSGSTLVE |
| | SINGFEAEFNKSQTSFHLRKPSVHISDTAEYFCAVHEIQG |
| | AQKLVFGQGTRLTINPNIQNPDPAVYQLRDSKSSDKSV |
| | CLFTDFDSQTNVSQSKDSDVYITDKTVLDMRSMDFKSN |
| | SAVAWSNKSDFACANAFNNSIIPEDTFFPSPESSCDVKL |
| | VEKSFETDTNLNFQNLSVIGFRILLLKVAGFNLLMTLRL |
| | WSS (SEQ ID NO: 50) |
| TCR1-beta-complete gene | MGTSLLCWVVLGFLGTDHTGAGVSQSPRYKVTKRGQD |
| | VALRCDPISGHVSLYWYRQALGQGPEFLTYFNYEAQQ |
| | DKSGLPNDRFSAERPEGSISTLTIQRTEQRDSAMYRCAS |
| | SFGVSYEQYFGPGTRLTVTEDLKNVFPPEVAVFEPSEAE |
| | ISHTQKATLVCLATGFYPDHVELSWWVNGKEVHSGVS |
| | TDPQPLKEQPALNDSRYCLSSRLRVSATFWQNPRNHFR |
| | CQVQFYGLSENDEWTQDRAKPVTQIVSAEAWGRADCG |
| | FTSESYQQGVLSATILYEILLGKATLYAVLVSALVLMA |
| | MVKRKDSRG (SEQ ID NO: 51) |
| TCR2-alpha-complete gene | MSLSSLLKVVTASLWLGPGIAQKITQTQPGMFVQEKEA |
| | VTLDCTYDTSDPSYGLFWYKQPSSGEMIFLIYQGSYDQ |
| | QNATEGRYSLNFQKARKSANLVISASQLGDSAMYFCA |
| | MRPLGGYNKLIFGAGTRLAVHPYIQNPDPAVYQLRDSK |
| | SSDKSVCLFTDFDSQTNVSQSKDSDVYITDKTVLDMRS |
| | MDFKSNSAVAWSNKSDFACANAFNNSIIPEDTFFPSPES |
| | SCDVKLVEKSFETDTNLNFQNLSVIGFRILLLKVAGFNL |
| | LMTLRLWSS (SEQ ID NO: 52) |
| TCR2-beta-complete gene | MGCRLLCCAVLCLLGAVPIDTEVTQTPKHLVMGMTNK |
| | KSLKCEQHMGHRAMYWYKQKAKKPPELMFVYSYEKL |
| | SINESVPSRFSPECPNSSLLNLHLHALQPEDSALYLCASS |
| | QAANEQFFGPGTRLTVLEDLKNVFPPEVAVFEPSEAEIS |
| | HTQKATLVCLATGFYPDHVELSWWVNGKEVHSGVSTD |
| | PQPLKEQPALNDSRYCLSSRLRVSATFWQNPRNHFRCQ |
| | VQFYGLSENDEWTQDRAKPVTQIVSAEAWGRADCGFT |
| | SESYQQGVLSATILYEILLGKATLYAVLVSALVLMAMV |
| | KRKDSRG (SEQ ID NO: 53) |
| TCR3-alpha-complete gene | MKTFAGFSFLFLWLQLDCMSRGEDVEQSLFLSVREGDS |
| | SVINCTYTDSSSTYLYWYKQEPGAGLQLLTYIFSNMDM |
| | KQDQRLTVLLNKKDKHLSLRIADTQTGDSAIYFCAEEG |
| | DRDYKLSFGAGTTVTVRANIQNPDPAVYQLRDSKSSDK |
| | SVCLFTDFDSQTNVSQSKDSDVYITDKTVLDMRSMDFK |
| | SNSAVAWSNKSDFACANAFNNSIIPEDTFFPSPESSCDV |
| | KLVEKSFETDTNLNFQNLSVIGFRILLLKVAGFNLLMTL |
| | RLWSS (SEQ ID NO: 54) |
| TCR3-beta-complete gene | EDLKNVFPPEVAVFEPSEAEISHTQKATLVCLATGFYPD |
| | HVELSWWVNGKEVHSGVSTDPQPLKEQPALNDSRYCL |
| | SSRLRVSATFWQNPRNHFRCQVQFYGLSENDEWTQDR |
| | AKPVTQIVSAEAWGRADCGFTSESYQQGVLSATILYEIL |
| | LGKATLYAVLVSALVLMAMVKRKDSRG (SEQ ID |
| | NO: 55) |
| TCR1-alpha constant | NIQNPDPAVYQLRDSKSSDKSVCLFTDFDSQTNVSQSK |
| | DSDVYITDKTVLDMRSMDFKSNSAVAWSNKSDFACAN |
| | AFNNSIIPEDTFFPSPESSCDVKLVEKSFETDTNLNFQNL |
| | SVIGFRILLLKVAGFNLLMTLRLWSS (SEQ ID NO: 56) |
| TCR1-beta constant (beta | DLKNVFPPEVAVFEPSEAEISHTQKATLVCLATGFYPDH |
| chain of NYESO TCR in | VELSWWVNGKEVHSGVSTDPQPLKEQPALNDSRYCLss |
| lowercase) | rlrvsatfwqnprnhfrcqvqfyglsendewtqdrakpvtqiv |
| | saeawgradcgftsesyqqgvlsatilyeillgkatlyavlv |
| | salvlmamvkrkdsrg (SEQ ID NO: 57) |
| TCR2-alpha constant | IQNPDPAVYQLRDSKSSDKSVCLFTDFDSQTNVSQSKDS |
| | DVYITDKTVLDMRSMDFKSNSAVAWSNKSDFACANAF |
| | NNSIIPEDTFFPSPESSCDVKLVEKSFETDTNLNFQNLSVI |
| | GFRILLLKVAGFNLLMTLRLWSS (SEQ ID NO: 58) |
| TCR2-beta constant | EDLKNVFPPEVAVFEPSEAEISHTQKATLVCLATGFYPD |
| | HVELSWWVNGKEVHSGVSTDPQPLKEQPALNDSRYCL |
| | SSRLRVSATFWQNPRNHFRCQVQFYGLSENDEWTQDR |
| | AKPVTQIVSAEAWGRADCGFTSESYQQGVLSATILYEIL |
| | LGKATLYAVLVSALVLMAMVKRKDSRG (SEQ ID |
| | NO: 59) |
| TCR3-alpha constant | IQNPDPAVYQLRDSKSSDKSVCLFTDFDSQTNVSQSKDS |
| | DVYITDKTVLDMRSMDFKSNSAVAWSNKSDFACANAF |
| | NNSIIPEDTFFPSPESSCDVKLVEKSFETDTNLNFQNLSVI |
| | GFRILLLKVAGFNLLMTLRLWSS (SEQ ID NO: 60) |
| TCR3-beta constant | EDLKNVFPPEVAVFEPSEAEISHTQKATLVCLATGFYPD |
| | HVELSWWVNGKEVHSGVSTDPQPLKEQPALNDSRYCL |
| | SSRLRVSATFWQNPRNHFRCQVQFYGLSENDEWTQDR |
| | AKPVTQIVSAEAWGRADCGFTSESYQQGVLSATILYEIL |
| | LGKATLYAVLVSALVLMAMVKRKDSRG (SEQ ID |
| | NO: 61) |

### IV. ANTIBODIES

Aspects of the disclosure relate to antibodies that target the peptides of the disclosure, or fragments thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ϵ) chains, respectively. IgG has several subtypes, including, but not limited to, IgG1, IgG2, IgG3, and IgG4. IgM subtypes include IgM1 and IgM2. IgA subtypes include IgA1 and IgA2.

1. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as H1, H2 and H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, Aug. 1970); Chothia (as described in C. Chothia et al., “Conformations of immunoglobulin hypervariable regions,” Nature, vol. 342, no. 6252, pp. 877-883, Dec. 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope. 2) Hydrogen-deuterium exchange and mass spectroscopy 3) Polypeptide fragmentation and peptide mapping approaches in which one generates multiple overlapping peptide fragments from the full length of the polypeptide and evaluates the binding affinity of these peptides for the epitope. 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

B. Functional Antibody Fragments and Antigen-Binding Fragments

1. Antigen-Binding Fragments

Certain aspects relate to antibody fragments, such as antibody fragments that bind to a peptide of the disclosure. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, N.Y. (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

2. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

C. Polypeptides with antibody CDRs & Scaffolding Domains that Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

D. Antibody Binding

The term “selective binding agent” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20- fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful in certain embodiments may have an affinity constant (Ka) of about, at least about, or at most about 10⁶, 10⁷, 10⁸, 10⁹, or 10¹⁰ M or any range derivable therein. Similarly, in some embodiments, antibodies may have a dissociation constant of about, at least about or at most about 10⁻⁶, 10⁻⁷, 10⁻⁸, 10⁻⁹, 10⁻¹⁰ M, or any range derivable therein. These values are reported for antibodies discussed herein and the same assay may be used to evaluate the binding properties of such antibodies. An antibody of the invention is said to “specifically bind” its target antigen when the dissociation constant (KD) is ≤10⁻⁸ M. The antibody specifically binds antigen with “high affinity” when the KD is ≤5×10⁻⁹ M, and with “very high affinity” when the KD is ≤5×10⁻¹⁰ M.

2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of about 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

3. Modification of Antibody Antigen-Binding Domains

It is understood that the antibodies of the present invention may be modified, such that they are substantially identical to the antibody polypeptide sequences, or fragments thereof, and still bind the epitopes of the present invention. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present invention, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with VH and VL region pairs that bind either the same antigen (multi-valent) or a different antigen (multi-specific).

### V. PROTEINACEOUS COMPOSITIONS

As used herein, a “protein” “peptide” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.).

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with, with at least, or with at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID Nos:1-1403. In specific embodiments, the peptide or polypeptide is or is based on a human sequence. In certain embodiments, the peptide or polypeptide is not naturally occurring and/or is in a combination of peptides or polypeptides.

In some embodiments, a peptide or polypeptide described herein comprises, comprises at least, or comprises at most 1, 2, 3, 4, 5, 6, 7, 8, 9, 10, 11, 12, 13, 14, 15, 16, 17, 18, 19, or 20 substitutions (or any derivable range therein) at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 (or any range derivable therein) of SEQ ID NOS:1-1403. In some embodiments, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 of a peptide or polypeptide of SEQ ID NO:1-1403 is substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some embodiments, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOs:1-1403.

In some embodiments, the protein, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs:1-1403.

In some embodiments, the polypeptide, protein, or nucleic acid may comprise, comprise at least, comprises at most, or comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID Nos:1-1403 that are, are at least, are at most, are exactly, or are about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS:1-1403.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS:1-1403 and comprising, comprising at least, comprising at most, or comprising about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS:1-1403.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein structure.

In some embodiments of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### VI. NUCLEIC ACIDS

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double- stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial sequence identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

1. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5x SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

1. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

2. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. See, eg., Gaily Kivi et al., BMC Biotechnol. 16:2 (2016). In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or part of one or more of the CDRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### VII. ANTIBODY PRODUCTION

A. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745 (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques. See, for example, Stewart and Young, (1984); Tarn et al, (1983); Merrifield, (1986); and Barany and Merrifield (1979), each incorporated herein by reference.

Briefly, a polyclonal antibody is prepared by immunizing an animal with an antigen or a portion thereof and collecting antisera from that immunized animal. The antigen may be altered compared to an antigen sequence found in nature. In some embodiments, a variant or altered antigenic peptide or polypeptide is employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography (Harlow and Lane, Antibodies: A Laboratory Manual 1988).

Methods of making monoclonal antibodies are also well known in the art (Kohler and Milstein, 1975; Harlow and Lane, 1988, U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after about two to three weeks) for the desired reactivity. Fusion procedures for making hybridomas, immunization protocols, and techniques for isolation of immunized splenocytes for fusion are known in the art.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA 93:7843-7848 (1996), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001).

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with embodiments include, but are not limited to, IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m2) (Johnson/Mead, N.J.), cytokines such as β-interferon, IL-2, or IL-12, or genes encoding proteins involved in immune helper functions, such as B-7.A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

B. Fully Human Antibody Production

Methods are available for making fully human antibodies. Using fully human antibodies can minimize the immunogenic and allergic responses that may be caused by administering non-human monoclonal antibodies to humans as therapeutic agents. In one embodiment, human antibodies may be produced in a non-human transgenic animal, e.g., a transgenic mouse capable of producing multiple isotypes of human antibodies to protein (e.g., IgG, IgA, and/or IgE) by undergoing V-D-J recombination and isotype switching. Accordingly, this aspect applies to antibodies, antibody fragments, and pharmaceutical compositions thereof, but also non-human transgenic animals, B-cells, host cells, and hybridomas that produce monoclonal antibodies. Applications of humanized antibodies include, but are not limited to, detect a cell expressing an anticipated protein, either in vivo or in vitro, pharmaceutical preparations containing the antibodies of the present invention, and methods of treating disorders by administering the antibodies.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc. Natl. Acad. Sci. USA 90:2551-2555 (1993); Jakobovits et al., Nature 362:255-258 (1993); Bruggermann et al., Year in Immunol. 7:33 (1993). In one example, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then crossbred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, International Patent Application Publication Nos. WO 96/33735 and WO 94/02602, which are hereby incorporated by reference in their entirety. Additional methods relating to transgenic mice for making human antibodies are described in U.S. Pat. Nos. 5,545,807; 6,713,610; 6,673,986; 6,162,963; 6,300,129; 6,255,458; 5,877,397; 5,874,299 and 5,545,806; in International Patent Application Publication Nos. WO 91/10741 and WO 90/04036; and in European Patent Nos. EP 546073B1 and EP 546073A1, all of which are hereby incorporated by reference in their entirety for all purposes.

The transgenic mice described above, referred to herein as “HuMAb” mice, contain a human immunoglobulin gene minilocus that encodes unrearranged human heavy (μ and γ) and κ light chain immunoglobulin sequences, together with targeted mutations that inactivate the endogenous μ and κ chain loci (Lonberg et al., Nature 368:856-859 (1994)). Accordingly, the mice exhibit reduced expression of mouse IgM or κ chains and in response to immunization, the introduced human heavy and light chain transgenes undergo class switching and somatic mutation to generate high affinity human IgG κ monoclonal antibodies (Lonberg et al., supra; Lonberg and Huszar, Intern. Ref Immunol. 13:65-93 (1995); Harding and Lonberg, Ann. N.Y. Acad. Sci. 764:536-546 (1995)). The preparation of HuMAb mice is described in detail in Taylor et al., Nucl. Acids Res. 20:6287-6295 (1992); Chen et al., Int. Immunol. 5:647-656 (1993); Tuaillon et al., J. Immunol. 152:2912-2920 (1994); Lonberg et al., supra; Lonberg, Handbook of Exp. Pharmacol. 113:49-101 (1994); Taylor et al., Int. Immunol. 6:579-591 (1994); Lonberg and Huszar, Intern. Ref. Immunol. 13:65-93 (1995); Harding and Lonberg, Ann. N.Y. Acad. Sci. 764:536-546 (1995); Fishwild et al., Nat. Biotechnol. 14:845-851 (1996); the foregoing references are herein incorporated by reference in their entirety for all purposes. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807; as well as International Patent Application Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., Nat. Genetics 15:146-156 (1997), which are herein incorporated by reference. For example, the HCo7 and HCo12 transgenic mice strains can be used to generate human antibodies.

Using hybridoma technology, antigen-specific humanized monoclonal antibodies with the desired specificity can be produced and selected from the transgenic mice such as those described above. Such antibodies may be cloned and expressed using a suitable vector and host cell, or the antibodies can be harvested from cultured hybridoma cells. Fully human antibodies can also be derived from phage-display libraries (as disclosed in Hoogenboom et al., J. Mol. Biol. 227:381 (1991); and Marks et al., J. Mol. Biol. 222:581 (1991)). One such technique is described in International Patent Application Publication No. WO 99/10494 (herein incorporated by reference), which describes the isolation of high affinity and functional agonistic antibodies for MPL- and msk-receptors using such an approach.

C. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980).

Single-chain variable fragments (scFvs) may be prepared by fusing DNA encoding a peptide linker between DNAs encoding the two variable domain polypeptides (VL and VH). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001)). By combining different VL- and VH-comprising polypeptides, one can form multimeric scFvs that bind to different epitopes (Kriangkum et al., Biomol. Eng. 18:31-40 (2001)). Antigen-binding fragments are typically produced by recombinant DNA methods known to those skilled in the art. Although the two domains of the Fv fragment, VL and VH, are coded for by separate genes, they can be joined using recombinant methods by a synthetic linker that enables them to be made as a single chain polypeptide (known as single chain Fv (sFv or scFv); see e.g., Bird et al., Science 242:423-426 (1988); and Huston et al., Proc. Natl. Acad. Sci. USA 85:5879-5883 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by amino-terminal and/or carboxy-terminal deletions, where the remaining amino acid sequence is substantially identical to the corresponding positions in the naturally occurring sequence deduced, for example, from a full-length cDNA sequence.

Antibodies may also be generated using peptide analogs of the epitopic determinants disclosed herein, which may consist of non-peptide compounds having properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987). Liu et al. (2003) also describe “antibody like binding peptidomimetics” (ABiPs), which are peptides that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the invention are proteins that are structurally similar to an antibody displaying a desired biological activity, such as the ability to bind a protein, but have one or more peptide linkages optionally replaced by a linkage selected from: —CH2NH—, —CH2S—, —CH2—CH2—, —CH=CH— (cis and trans), —COCH2—, —CH(OH)CH2—, and —CH2SO— by methods well known in the art. Systematic substitution of one or more amino acids of a consensus sequence with a D-amino acid of the same type (e.g., D-lysine in place of L-lysine) may be used in certain embodiments of the invention to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of the Fab molecules using known techniques. See, e.g., Figini et al., J. Mol. Biol. 239:68 (1994). The coding sequences for the heavy and light chain portions of the Fab molecules selected from the phage display library can be isolated or synthesized and cloned into any suitable vector or replicon for expression. Any suitable expression system can be used.

VIII. Polypeptide Expression

In some aspects, there are nucleic acid molecule encoding polypeptides or peptides of the disclosure (e.g antibodies, TCR genes, and immunogenic peptides). These may be generated by methods known in the art, e.g., isolated from B cells of mice that have been immunized and isolated, phage display, expressed in any suitable recombinant expression system and allowed to assemble to form antibody molecules or by recombinant methods.

1. Expression

The nucleic acid molecules may be used to express large quantities of polypeptides. If the nucleic acid molecules are derived from a non-human, non-transgenic animal, the nucleic acid molecules may be used for humanization of the antibody or TCR genes.

2. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. In some aspects, a vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

3. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

4. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624,5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

5. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor- targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

B. Isolation

The nucleic acid molecule encoding either or both of the entire heavy and light chains of an antibody or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

IX. Additional Therapies

A. Immunotherapy

In some embodiments, the methods comprise administration of an additional therapy. In some embodiments, the additional therapy comprises a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

1. Checkpoint Inhibitors and Combination Treatment

Embodiments of the disclosure may include administration of immune checkpoint inhibitors, which are further described below.

1. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP- 224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX- 010, MDX- 101, and Yervoy®) or antigen binding fragments and variants thereof (see, e.g., WO0 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above- mentioned antibodies. In another embodiment, the antibody has at least about 70, 75, 80, 85, 90, 95, 97, or 99% (or any derivable range therein) variable region amino acid sequence identity with the above-mentioned antibodies.

2. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

2. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

3. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

5. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death. [60]

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

B. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-a), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

C. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

D. Surgery

In some embodiments, the additional therapy comprises surgery. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months (or any range derivable therein). These treatments may be of varying dosages as well.

### X. FORMULATIONS AND CULTURE OF THE CELLS

In particular embodiments, the cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HC1; Linoleic Acid; Linolenic Acid; Progesterone; Putrescine 2HCl; Sodium Selenite; and/or T3 (triodo-I-thyronine). . In specific embodiments, one or more of these may be explicitly excluded.

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a ^{B27}® supplement, xeno-free ^{B27}® supplement, GS21™ supplement, or combinations thereof. In some embodiments, the medium comprises or futher comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific embodiments, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. . In specific embodiments, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific embodiments, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

In some embodiments, the method further comprises priming the T cells. In some embodiments, the T cells are primed with antigen presenting cells. In some embodiments, the antigen presenting cells present tumor antigens or peptides, such as those disclosed herein.

In particular embodiments, the cells of the disclosure comprise an exogenous TCR, which may be of a defined antigen specificity. In some embodiments, the TCR can be selected based on absent or reduced alloreactivity to the intended recipient (examples include certain virus-specific TCRs, xeno-specific TCRs, or cancer-testis antigen-specific TCRs). In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some embodiments, the choice of exogenous TCR may not necessarily be defined based on lack of alloreactivity. In some embodiments, the endogenous TCR genes have been modified by genome editing so that they do not express a protein. Methods of gene editing such as methods using the CRISPR/Cas9 system are known in the art and described herein.

In some embodiments, the cells of the disclosure further comprise one or more chimeric antigen receptors (CARs). Examples of tumor cell antigens to which a CAR may be directed include at least 5T4, 8H9, integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRvIII, EGP2, EGP40, ERBB3, ERBB4, ErbB3/4, EPCAM, EphA2, EpCAM, folate receptor-a, FAP, FBP, fetal AchR, FRα, GD2, G250/CAIX, GD3, Glypican-3 (GPC3), Her2, IL-13Rα2, Lambda, Lewis-Y, Kappa, KDR, MAGE, MCSP, Mesothelin, Muc1, Muc16, NCAM, NKG2D Ligands, NY-ESO-1, PRAIVIE, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, or VEGF receptors (e.g., VEGFR2), for example. The CAR may be a first, second, third, or more generation CAR. The CAR may be bispecific for any two nonidentical antigens, or it may be specific for more than two nonidentical antigens.

### XI. ADMINISTRATION OF THERAPEUTIC COMPOSITIONS

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μ; or about 10 μM to 150 μ; or about 10 μM to 100 μ; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

XII. Kits

Certain aspects of the present invention also concern kits containing compositions of the disclosure or compositions to implement methods of the invention. In some embodiments, kits can be used to evaluate one or more biomarkers or HLA types. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 2×, 5×, 10×, or 20× or more.

In certain aspects, negative and/or positive control nucleic acids, probes, and inhibitors are included in some kit embodiments. In addition, a kit may include a sample that is a negative or positive control for methylation of one or more biomarkers.

It is contemplated that any method or composition described herein can be implemented with respect to any other method or composition described herein and that different embodiments may be combined. The claims originally filed are contemplated to cover claims that are multiply dependent on any filed claim or combination of filed claims.

### XIII. SEQUENCES

E3 ubiquitin-protein ligase TRIM11 isoform X2 [Homo sapiens]; NCBI Reference
Sequence:XP_016857901.1:
MAAPDLSTNLQEEATCAICLDYFTDPVMTDCGHNFCRECIRRCWGQPEGPYACPEC
RELSPQRNLRPNRPLAKMAEMARRLHPPSPVPQGVCPAHREPLAAFCGDELRLLCAA
CERSGEHWAHRVRPLQDAAEDLKAKLEKSLEHLRKQMQDALLFQAQADETCVLW
QDIKDALRRVQDVKLQPPEVVPMELRTVCRVPGLVETLRRFRGDVTLDPDTANPELI
LSEDRRSVQRGDLRQALPDSPERFDPGPCVLGQERFTSGRHYWEVEVGDRTSWALG
VCRENVNRKEKGELSAGNGFWILVFLGSYYNSSERALAPLRDPPRRVGIFLDYEAGH
LSFYSATDGSLLFIFPEIPFSGTLRPLFSPLSSSPTPMTICRPKGGSGDTLAPQ (SEQ ID
NO: 1)
Peptide embodiments of the TRIM11 gene comprise: DETCVLWQD (SEQ ID
NO: 7), VLWQDIKDAL (SEQ ID NO: 8), and LWQDIKDAL (SEQ ID NO: 9)
RCOR3 protein [Homo sapiens]; GenBank: AAH31608.1:
MPGMMEKGPELLGKNRSANGSAKSPAGGGGSGASSTNGGLHYSEPESGCSSDDEHD
VGMRVGAEYQARIPEFDPGATKYTDKDNGGMLVWSPYHSIPDARLDEYIAIAKEKH
GYNVEQALGMLFWHKHNIEKSLADLPNFTPFPDEWTVEDKVLFEQAFSFHGKSFHRI
QQMLPDKTIASLVKYYYSWKKTRSRTSLMDRQARKLANRHNQGDSDDDVEETHPM
DGNDSDYDPKKEAKKEGNTEQPVQTSKIGLGRREYQSLQHRHHSQRSKCRPPKGMY
LTQEDVVAVSCSPNAANTILRQLDMELISLKRQVQNAKQVNSALKQKMEGGIEEFKP
PESNQKINARWTTEEQLLAVOGTDPTGSSDTGSITSCPIIHSNTNSPYCHSEPASTTSSS
NTACCPGSSPAASSTPAAGSVHPAPANFKSASTTSYSPC (SEQ ID NO: 2)
Peptide embodiments of the RCOR3 gene comprise: LAVQGTDPT (SEQ ID NO: 10).
Protein FAM76B isoform 1 [Homo sapiens]; NCBI Reference Sequence:
NP_653265.3:
MAASALYACTKCTQRYPFEELSQGQQLCKECRIAHPIVKCTYCRSEFQQESKTNTICK
KCAQNVKQFGTPKPCQYCNIIAAFIGTKCQRCTNSEKKYGPPQTCEQCKQQCAFDRK
EEGRRKVDGKLLCWLCTLSYKRVLQKTKEQRKSLGSSHSNSSSSSLTEKDQHHPKH
HHHHHHHHHRHSSSHHKISNLSPEEEQGLWKQSHKSSATIQNETPKKKPKLESKPSN
GDSSSINQSADSGGTDNFVLISQLKEEVMSLKRLLQQRDQTILEKDKKLTELKADFQY
QESNLRTKMNSMEKAHKETVEQLQAKNRELLKQVAALSKGKKFDKSGSILTSP
(SEQ ID NO: 3)
Peptide embodiments of the FAM76B gene comprise: PSNGDSSSI (SEQ ID NO: 11)
and KPSNGDSSSI (SEQ ID NO: 12)
Sarcolemmal membrane-associated protein isoform f [Homo sapiens]; NCBI
Reference Sequence: NP_001298107.1:
MDEQDLNEPLAKVSLLKDDLQGAQSEIEAKQEIQHLRKELIEAQELARTSKQKCFEL
QALLEEERKAYRNQVEESTKQIQVLQAQLQRLHIDTENLREEKDSEITSTRDELLSAR
DEILLLHQAAAKVASERDTDIASLQEELKKVRAELERWRKAASEYEKEITSLQNSFQL
RCQQCEDQQREEATRLQGELEKLRKEWNALETECHSLKRENVLLSSELQRQEKELH
NSQKQSLELTSDLSILQMSRKELENQVGSLKEQHLRDSADLKTLLSKAENQAKDVQK
EYEKTQTVLSELKLKFEMTEQEKQSITDELKQCKNNLKLLREKGNNPSILQPVPAVFI
GLFLAFLFWCFGPLW (SEQ ID NO: 4)
Peptide embodiments of the SLMAP gene comprise: NNPSILQPV (SEQ ID NO: 13),
REKGNNPSI (SEQ ID NO: 14), and REKGNNPSIL (SEQ ID NO: 15)
Transmembrane protein 62 isoform a [Homo sapiens]; NCBI Reference Sequence:
P_079232.3:
MAAVLALRVVAGLAAAALVAMLLEHYGLAGQPSPLPRPAPPRRPHPAPGP
GDSNIFWGLQISDIHLSRFRDPGRAVDLEKFCSETIDIIQPALVLATGDLTDAKTKEQL
GSRQHEVEWQTYQGILKKTRVMEKTKWLDIKGNHDAFNIPSLDSIKNYYRKYSAVR
RDGSFHYVHSTPFGNYSFICVDATVNPGPKRPYNFFGILDKKKMEELLLLAKESSRSN
HTIWFGHFTTSTILSPSPGIRSIMSSAIAYLCGHLHTLGGLMPVLHTRHFQGTLELEVG
DWKDNRRYRIFAFDHDLFSFADLIFGKWPVVLITNPKSLLYSCGEHEPLERLLHSTHI
RVLAFSLSSITSVTVKIDGVHLGQAVHVSGPIFVLKWNPRNYSSGTHNIEVIVQDSAG
RSKSVHHIFSVQENNHLSFDPLASFILRTDHYIMARVLFVLIVLSQLTILIIFRYRGYPE
LKEPSGFINLTSFSLHVLSKINIFYYSVLLLTLYTVLGPWFFGEIIDGKFGCCFSFGIFVN
GHFLQGSITFIIGILQLAFFNIPLMAYMCWSLLQRCFGHNFRSHLHQRKYLKIMPVHL
LMLLLYIWQVYSCYFLYATYGTLAFLFSPLRTWLTLLTPVLIRYVWTLNSTKFGIFM
VQLKSHLSS (SEQ ID NO: 5)
Peptide embodiments of the TMEM62 gene comprise: YTVLGPWFF (SEQ ID
NO: 16), TLYTVLGPW (SEQ ID NO: 17), TLYTVLGPWF (SEQ ID NO: 18), LYTVLGPWF
(SEQ ID NO: 19), LTLYTVLGPW (SEQ ID NO:20), LYTVLGPWFF (SEQ ID NO: 21), and
VLGPWFFGEI (SEQ ID NO: 22).
PLA2G6 [Homo sapiens]; GenBank: CAG30429.1:
MQFFGRLVNTF SGVTNLF SNPFRVKEVAVAD YTS SDRVREEGQLILFQNTPNRTWDC
VLVNPRNSQSGFRLFQLELEADALVNFHQYSSQLLPFYESSPQVLHTEVLQHLTDLIR
NHPSWSVAHLAVELGIRECFHHSRIISCANCAENEEGCTPLHLACRKGDGEILVELVQ
YCHTQMDVTDYKGETVFHYAVQGDNSQVLQLLGRNAVAGLNQVNNQGLTPLHLA
CQLGKQEMVRVLLLCNARCNIMGPNGYPIHSAMKFSQKGCAEMIISMDSSQIHSKDP
RYGASPLHWAKNAEMARMLLKRGCNVNSTSSAGNTALHVAVMRNRFDCAIVLLTH
GANADARGEHGNTPLHLAMSKDNVEMIKALIVFGAEVDTPNDFGETPTFLASKIGRL
VTRKAILTLLRTVGAEYCFPPIHGVPAEQGSAAPHHPFSLERAQPPPISLNNLELQDLM
HISRARKPAFILGSMRDEKRTHDHLLCLDGGGVKGLIIIQLLIAIEKASGVATKDLFD
WVAGTSTGGILALAILHSKSMAYMRGMYFRMKDEVFRGSRPYESGPLEEFLKREFG
EHTKMTDVRKPKVMLTGTLSDRQPAELHLFRNYDAPETVREPRFNQNVNLRPPAQP
SDQLVWRAARSSGAAPTYFRPNGRFLDGGLLANNPTLDAMTEIHEYNQDLIRKGQA
NKVKKLSIVVSLGTGRSPQVPVTCVDVFRPSNPWELAKTVFGAKELGKMVVDCCTD
PDGRAVDRARAWCEMVGIQYFRLNPQLGTDIMLDEVSDTVLVNALWETEVYIYEHR
EEFQKLIQLLLSP (SEQ ID NO: 6)
Peptide embodiments of the TMEM62 gene comprise: TFLASKIGRLV (SEQ ID
NO: 23), RLVTRKAIL (SEQ ID NO: 24), FLASKIGRL (SEQ ID NO: 25), SKIGRLVTRK
(SEQ ID NO: 26), FLASKIGRLV (SEQ ID NO: 27), LASKIGRLV (SEQ ID NO: 28), and
KIGRLVTRK (SEQ ID NO: 29).
TRA and TRB CDR3 sequences include those listed below: TRA-1 CDR3:
CAVHEIQGAQKLVF (SEQ ID NO: 30); TRB-1 CDR3: CASSFGVSYEQYF (SEQ ID
NO: 31); TRA-2 CDR3: CAMRPLGGYNKLIF (SEQ ID NO: 32); TRB-2 CDR3:
CASSQAANEQFF (SEQ ID NO: 33); TRA-3 CDR3: CAEEGDRDYKLSF (SEQ ID NO: 34);
TRB-3 CDR3: CASTGRSGRSEQYF (SEQ ID NO: 35); TRA-4 CDR3: CAFMKGRDDKIIF
(SEQ ID NO: 36); TRB-4 CDR3: CATTLPGDTEAFF (SEQ ID NO: 37); TRA-5 CDR3:
CATANNAGNMLTF(SEQ ID NO: 38); TRB-5 CDR3: CASSLDRHQPQHF (SEQ ID
NO: 39); TRA-6 CDR3: CALWEGQGGSEKLVF (SEQ ID NO: 40); TRB-6 CDR3:
CASSLEARAPSGNTIYF (SEQ ID NO: 41); and TRA-7 CDR3: CAVGAGTGTASKLTF
(SEQ ID NO: 42); TRB-7 CDR3: CASSLELAGGRDTQYF (SEQ ID NO: 43).

Additional peptide embodiments include those below:

| Peptide Sequence | HLA | SEQ ID NO: |
|---|---|---|
| NTEPVKDPY | HLA-A*01:01 | 1364 |
| YTYQMHGEY | HLA-A*01:01 | 1365 |
| LLKIDSFELLY | HLA-A*01:01 | 1366 |
| KIDSFELLYY | HLA-A*01:01 | 1367 |
| LKIDSFELLYY | HLA-A*01:01 | 1368 |
| WLEAVYCGCY | HLA-A*01:01 | 1369 |
| WSVFSTSLY | HLA-A*01:01 | 1370 |
| NLLTTCSTV | HLA-A*02:01 | 1371 |
| FTVTVTEPL | HLA-A*02:01 | 1372 |
| YLEAKADLV | HLA-A*02:01 | 1373 |
| YLDQLNHILA | HLA-A*02:01 | 1374 |
| FLQEPLQVFNV | HLA-A*02:01 | 1375 |
| FLPRGTPAL | HLA-A*02:01 | 1376 |
| RMAEHHSFWV | HLA-A*02:01 | 1377 |
| HLLRIFCTI | HLA-A*02:01 | 1378 |
| SLSEVDIPSI | HLA-A*02:01 | 1379 |
| LILKGIFCTI | HLA-A*02:01 | 1380 |
| STWGGFDEL | HLA-A*02:01 | 1381 |
| TVTEPLLVK | HLA-A*03:01 | 1382 |
| GLQTRAFWK | HLA-A*03:01 | 1383 |
| ILLYKNKRK | HLA-A*03:01 | 1384 |
| KGFSIDSGK | HLA-A*03:01 | 1395 |
| LLQGDTPVRK | HLA-A*03:01 | 1385 |
| SLDWETPSK | HLA-A*03:01 | 1386 |
| KEMRPARAK | HLA-A*03:01 | 1387 |
| RAVTNHSVYY | HLA-A*03:01 | 1388 |
| SVYYPSECSK | HLA-A*03:01 | 1389 |
| QLFEGMKAFK | HLA-A*03:01 | 1390 |
| ALGQLFEGMK | HLA-A*03:01 | 1391 |
| KMFRKLHNSY | HLA-A*03:01 | 1392 |
| RLQARPRLGR | HLA-A*03:01 | 1393 |
| RIPYKVVARR | HLA-A*03:01 | 1394 |
| ISWMKGVPGK | HLA-A*03:01 | 786 |
| KIDSFELLYY | HLA-A*03:01 | 1367 |
| TVASSCSSPT | HLA-A*68:01 | 1396 |
| YEQHNGVDGL | HLA-B*40:01 | 1397 |
| ILKGIFCTI | HLA-A*32:01 | 1398 |
| SGKSRPLPV | HLA-B*08:01 | 1399 |
| QPSGKSRPL | HLA-B*07:05 | 1400 |
| TQAPPPPERK | HLA-A* 11:01 | 1401 |
| SSDCRVSQI | HLA-C* 15:05 | 1402 |

### XIV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Tumor Antigens Arising from Alternative Splicing Events may be Targetable by Tumor Infiltrating Lymphocytes in Glioblastomas

Alternative splicing, the cellular process that converts premature mRNA to mature mRNA and allows for a single gene to produce multiple protein products, is frequently dysregulated in many cancers, including glioblastoma. However, along with non-synonymous mutations in the DNA, altered splicing mechanisms in cancers may produce novel tumor antigens that distinguish cancer cells from healthy cells and can thus be targeted by the immune system.

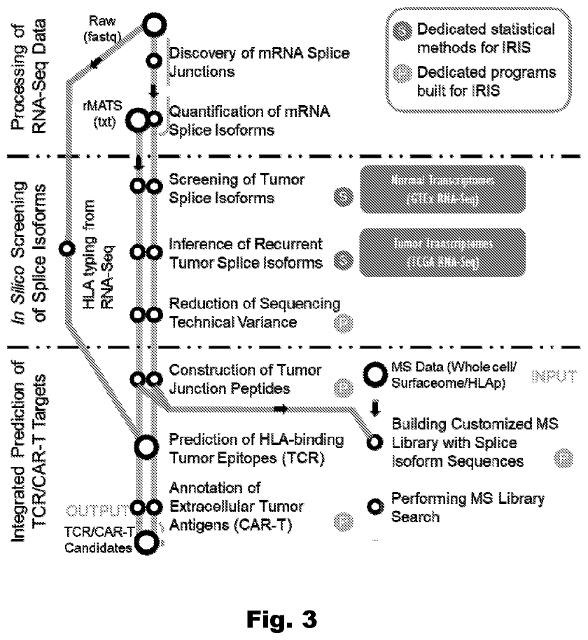

Provided in FIG. 3 is an exemplary computational pipeline to identify antigenic peptides from RNA data of neoplastic tissue. The computational pipeline, referred to Isoform peptides from RNA splicing for Immunotherapy target Screening (IRIS), is an integrated package. As can be seen, the process has three major modules: (1) processing of RNA-Seq data, (2) in silico screening of splice isoforms, and (3) integrated prediction of TCR/CAR-T targets.

The inventors used the IRIS (Isoform peptides from RNA splicing for Immunotherapy targets Screening) platform to take bulk RNA-sequencing data from 23 glioblastoma patient tumor samples and predict tumor antigens that may arise from alternative splicing events. Predicted tumor antigens that arose in HLA*A02:01 and HLA*A03:01 patients were prioritized and 8 potential tumor antigens were selected to generate peptide:MHC Class 1 dextramers. The inventors tested PBMCs and/or ex vivo expanded tumor infiltrating lymphocytes (TIL) from 6 glioblastoma patients against these dextramers, sorted for any tumor antigen-reactive T cells, and performed single-cell RNA sequencing on the sorted population to determine the TCR sequence.

Among the 8 predicted tumor antigens tested, 7 of the tumor antigens were recognized by at least 1 patient's T cells. 1 HLA*A03:01 epitope was recognized in 3 of the 4 HLA*A03 :01 patients and this epitope was highly positive in one of those patients' expanded TIL population, representing 1.7% of all CD3+CD8+cells. When the inventors sorted for those tumor antigens reactive T cells from the expanded TIL population and performed single-cell RNA sequencing, they found 325 unique T cell clonotypes, but the top 10 clonotypes represented 83.6% of all clonotypes, with the most frequent clonotype representing 39.1% of all clonotypes and indicating clonal expansion of a select few TCR clones from within the tumor.

In total, the data indicates that tumor antigens arising from alternative splicing events may represent a potential target for immunotherapy in glioblastoma.

### Example 2

### IRIS: Big Data-Informed Discovery of Cancer Immunotherapy Targets Arising from Pre-mRNA Alternative Splicing

Cancer immunotherapy has gained tremendous momentum in the past decade. The clinical effectiveness of checkpoint inhibitors, such as neutralizing antibodies against PD-1 and CTLA-4, is thought to result from their ability to reactivate tumor-specific T cells (1). Meanwhile, adoptive cell therapies use genetically modified T-cell receptors (TCRs) or synthetic chimeric antigen receptor T cells (CAR-T) for tumor-specific antigen recognition (2). The finding that cancer cells express specific T-cell-reactive antigens has galvanized epitope discovery in recent years (3-6). Nevertheless, the identification of tumor antigens remains a major challenge (7,8). Although somatic mutation-derived antigens have been successfully targeted by cancer therapies (9-12), this approach remains largely ineffective for tumors with low or moderate mutation loads (7-13).

Various types of dysregulation at the RNA level can generate immunogenic peptides in cancer cells (13-15). Notably, tumors harbor up to 30% more alternative splicing (AS) events than normal tissues, and the resulting peptides are predicted to be presented by human leukocyte antigen (HLA) (16). However, there are no integrated methods to systematically identify AS-derived tumor antigens. Therefore, the inventors leveraged tens of thousands of normal and tumor transcriptomes generated by large-scale consortium studies (e.g. GTEx, TCGA) (17,18) to build a versatile, big data-informed platform for discovering AS-derived immunotherapy targets. This in silico platform, named ‘IRIS’ (Isoform peptides from RNA splicing for Immunotherapy target Screening), incorporates three main components: processing of RNA-Seq data, in silico screening of tumor AS isoforms, and integrated prediction and prioritization of TCR and CAR-T targets (FIG. 3).

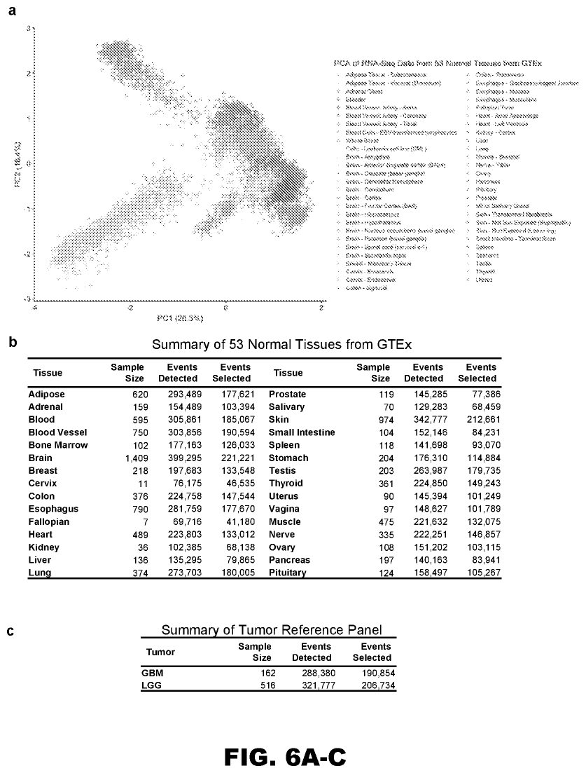

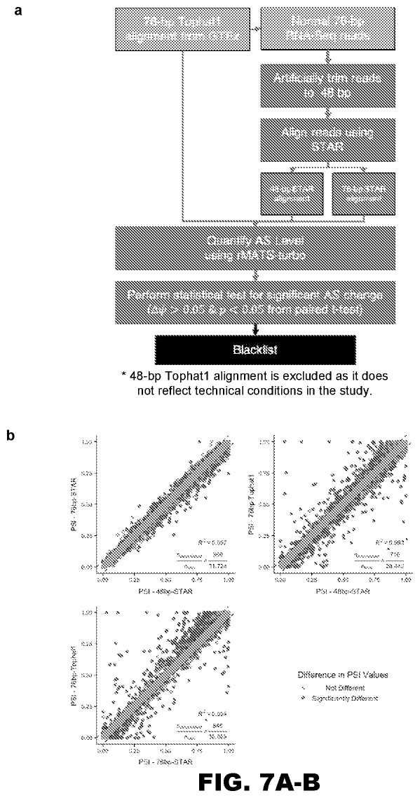

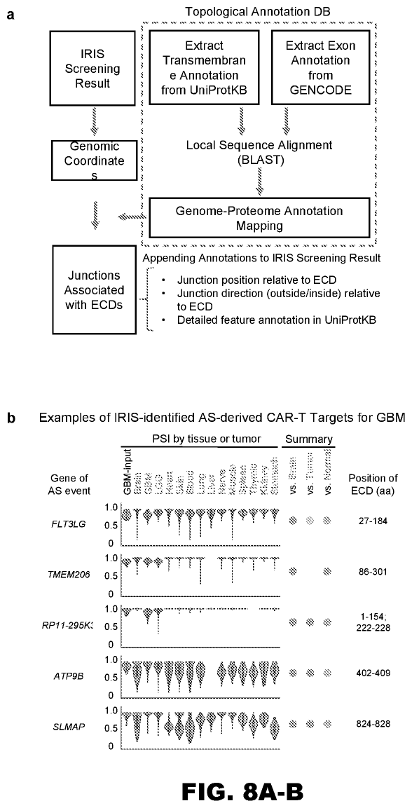

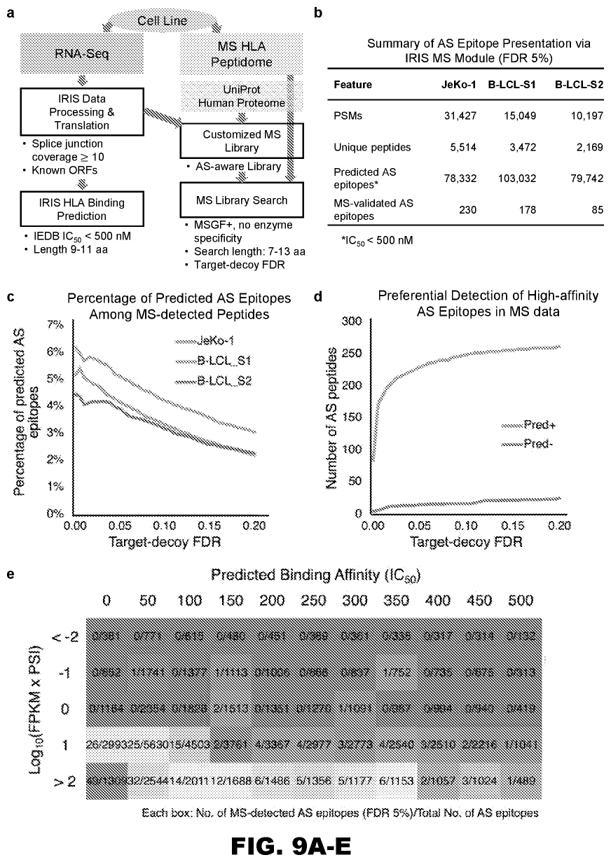

IRIS's RNA-Seq data-processing module uses standard input data to discover and quantify AS events in tumors using the ultra-fast rMATS-turbo software (19,20). Identified AS events are fed to the in silico screening module, which statistically compares AS events against any combination of events selected from large-scale (>10,000) reference RNA-Seq samples of normal and tumor tissues (FIG. 6) to identify AS events that are tumor-associated, tumor-recurrent, and potentially tumor-specific (Methods). Tumor specificity is a key metric for evaluating potential tissue toxicity, which is an important side effect of targeting lineage-specific antigens that are expressed by both tumor and normal cells (21). In addition to screening multiple patient samples simultaneously in the default ‘group mode’, IRIS can be performed in the ‘personalized mode’ to identify targets for a specific patient sample (Methods). Potential false-positive events are removed by using a blacklist of AS events whose quantification across diverse RNA-Seq datasets is error-prone due to technical variances such as read length (Methods and FIG. 7). IRIS's target prediction module first constructs splice-junction peptides of predicted tumor isoforms and then predicts AS-derived targets for TCR/CAR-T therapies (Methods). This module performs tumor HLA typing using RNA-Seq data and then integrates multiple HLA-binding prediction algorithms for predicting MR targets and/or peptide vaccines. In parallel, protein extracellular domain annotations are used for predicting CAR-T targets (FIG. 8). IRIS also includes the option to confirm predicted AS-derived targets using mass spectrometry (MS) data via proteo-transcriptomics data integration. This option provides an orthogonal approach for target discovery and validation by integrating RNA-Seq data with various types of MS data, such as whole-cell proteomics, surfaceomics, or immunopeptidomics data (Methods and FIG. 9A).

The inventors performed a proof-of-concept analysis and preliminary confirmation of AS-derived epitopes by applying IRIS to RNA-Seq and MS-based immunopeptidomics data of cancer and normal cell lines. The inventors identified hundreds of AS-derived epitopes that were supported by both RNA-Seq and MS data (FIG. 9B, Table 1). MS-supported epitopes were enriched for transcripts with high expression levels and peptides with strong predicted HLA-binding affinities (FIG. 9C-E), consistent with the expected pattern of HLA-epitope binding (22).

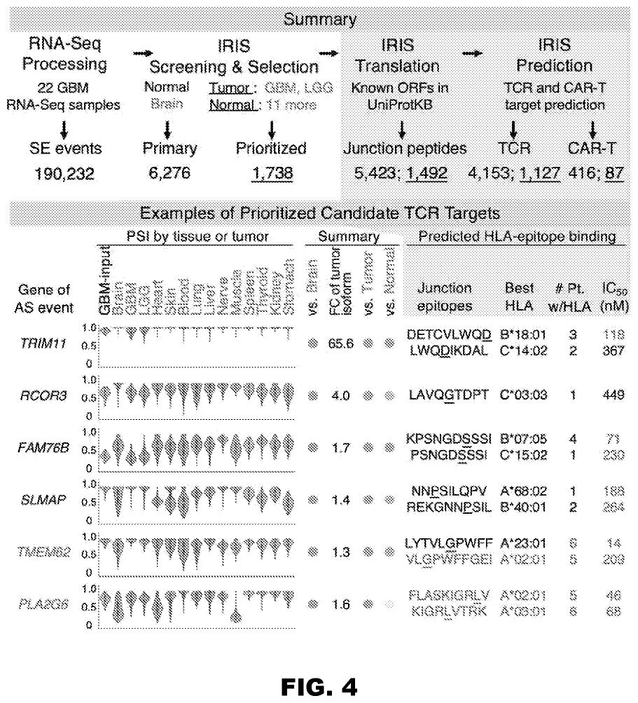

To explore IRIS's ability to discover AS-derived immunotherapy targets in clinical samples, the inventors generated RNA-Seq data from 22 resected glioblastomas (GBMs) and analyzed these data by IRIS. Candidate epitopes were then validated based on their recognition by patient T cells. FIG. 4 (top) summarizes the stepwise IRIS results. After uniform processing of RNA-Seq data by rMATS-turbo, IRIS discovered 190,232 putative skipped exon (SE) events from the 22 GBM samples. Using the in silico screening module, the inventors compared these AS events against reference normal and tumor panels to evaluate tumor association, recurrence, and specificity (Methods). Specifically, AS events were compared against: normal brain samples from GTEx (tissue-matched normal panel, for evaluating tumor association), two cohorts of brain tumor samples—GBM and lower-grade glioma (LGG)—from TCGA (tumor panel, for evaluating tumor recurrence), and 11 other selected normal (nonbrain) tissues from GTEx (normal panel, for evaluating tumor specificity). After initially screening against the tissue-matched normal panel and removing blacklisted events, IRIS identified 6,276 tumor-associated AS events in the 22 GBM samples (Primary' set, FIG. 4). Of these, 1,738 events were identified as tumor-recurrent and tumor-specific based on comparison with the tumor panel and normal panel, respectively (Prioritized' set, FIG. 4; Table 2).

Next, for each AS event, splice junctions of the tumor isoform (i.e. the isoform that was more abundant in the tumor samples than in the tissue-matched normal panel) were translated into peptides, followed by TCR/CAR-T target prediction (FIG. 4). For the GBM dataset, IRIS predicted 4,153 ‘primary’ tumor-associated epitope-producing splice junctions.

Of these, 1,127 were tumor-recurrent and tumor-specific compared to the tumor panel and normal panel, respectively, and were predicted to be ‘prioritized’ TCR targets. in parallel, IRIS identified 416 ‘primary’ tumor-associated extracellular peptide-producing splice junctions, of which 87 were predicted to be ‘prioritized’ CAR-T targets.

IRIS generates an integrative report for predicted immunotherapy targets (Table 3). Representative examples for six prioritized TCR targets are shown in the bottom panel of FIG. 4 (see FIG. 8B for CAR-T target examples). Violin plots depict exon inclusion levels across the 22 GBM samples (‘GBM-input’) and different sets of reference panels using the percent-spliced-in (PSI) metric (23). Tumor isoforms can be either the exon-skipped (low PSI) or the exon-included (high PSI) isoform compared to the tissue-matched normal panel. As illustrated by the darker dots in the ‘Summary’ column, all six epitope-producing splice junctions were tumor-associated compared to the tissue-matched normal panel (‘Brain’), and tumor-recurrent compared to the tumor panel (‘GBM’ and ‘IGG’). Two AS events (in TRIM1 1 and FAM76B) consistently showed distinct PSI values in tumors compared to normal brain and nonbrain tissues, indicating high tumor specificity. For candidate splice junctions, IRIS also calculates the fold-change (FC) of tumor isoforms between tumor samples and the tissue-matched normal panel (Methods). For example, the tumor isoform in TRIM11 had an average isoform proportion of 8.60% in the 22 GBM samples and 0.13% in normal brain samples, representing an FC of 65.6 in tumor samples versus the tissue-matched normal panel. The inventors note that, as shown under ‘Predicted HLA-epitope binding’, a single splice junction can give rise to multiple putative epitopes with distinct peptide sequences and HLA binding affinities.

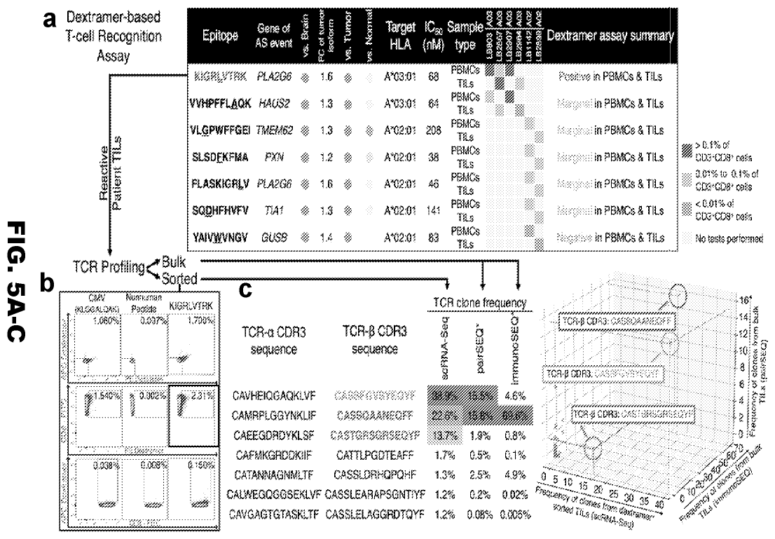

Finally, the inventors sought to validate the immunogenicity and T-cell recognition of IRIS-identified candidate TCR targets using an MHC class I dextramer-based assay (12,24). The inventors focused on predicted AS-derived tumor epitopes with strong putative HLA-binding affinity to common HLA types found in at least five of the 22 patients. The inventors selected seven AS-derived tumor-associated epitopes (five HLA-A02:01 and two HLA-A03:01) for dextramer-based T-cell recognition testing (Table 4). All but one epitope (YAIVWVNGV (SEQ ID NO:62)) showed some degree of tumor specificity when evaluated in normal (nonbrain) tissues (‘vs. Normal’, see FIG. 5A). The inventors obtained customized HLA-matched, fluorescently labeled MEW class I dextramer:peptide (pMHC) complexes for each candidate epitope. The inventors conducted flow cytometry to detect CD8⁺ T-cell binding with the pMHC complexes using available peripheral blood mononuclear cells (PBMCs) and/or ex vivo-expanded tumor-infiltrating lymphocytes (TILs). Based on the binding of each AS-derived tumor epitope to a patient's CD3⁺CD8⁺ T cells, the inventors classified epitope reactivity as ‘positive’ (binding>0.1% of cells), ‘marginal’ (binding 0.01-0.1% of cells), or ‘negative’ (binding<0.01% of cells). Epitopes that showed at least marginal reactivity were considered to be ‘recognized’ by patient T cells. The inventors analyzed samples from two HLA-A02:01 and four HLA-A03:01 patients, as well as samples from three HLA-A02:01 and three HLA-A03:01 healthy donors (Table 5, Supplementary Data).

Both predicted HLA-A03:01 tumor epitopes were recognized by patient T cells. In particular, one epitope (KIGRLVTRK (SEQ ID NO:29), in PLA2G6) was recognized by T cells from all four tested patients but only one of the three tested healthy donors. In one patient (LB2867), recognition of tumor epitope KIGRLVTRK (SEQ ID NO:29) was marginal in PBMCs but positive in the expanded TIL population, with epitope-reactive T cells representing 0.03% of T cells in PBMCs and 1.69% of T cells in TILs. This patient had been previously treated with neoadjuvant anti-PD-1 and anti-CTLA-4 checkpoint blockade immunotherapy. These results suggest epitope KIGRLVTRK (SEQ ID NO:29) as a promising immunotherapy target in HLA-A03 patients from the GBM cohort. T cells from another patient (LB2907) showed positive reactivity to both tested HLA-A03:01 epitopes. All four predicted HLA-A02:01 epitopes were recognized by T cells from tested patients and healthy donors. The non-tumor-specific epitope (YAIVWVNGV (SEQ ID NO:62), bottom row in FIG. 5A) was tested in two patients and three healthy donors and was recognized by T cells in only one healthy donor (marginal reactivity, 0.013% of CD3⁺CD8⁺ T cells). Taken together, the dextramer-based assay results indicate that the AS-derived TCR targets predicted by IRIS can be recognized by tumor-infiltrating and peripheral CD3⁺CD8⁺ T cells.

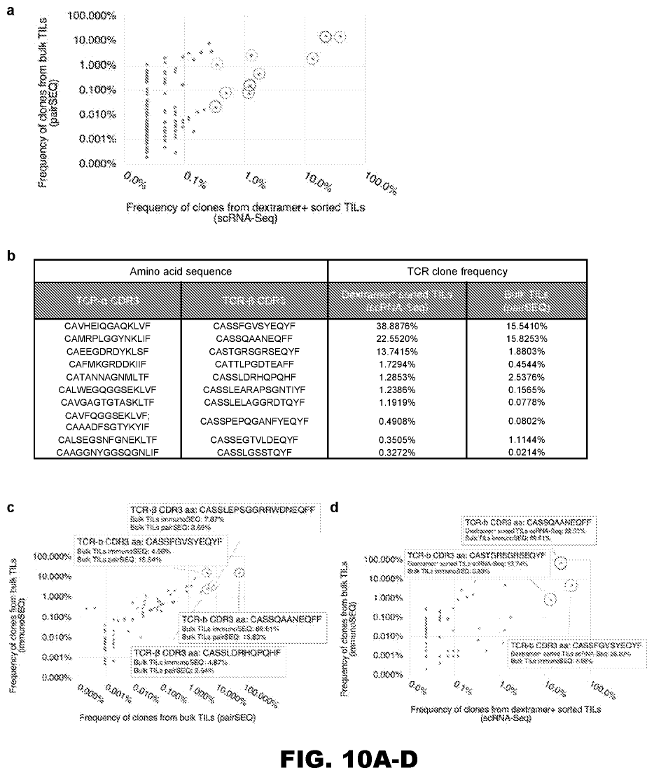

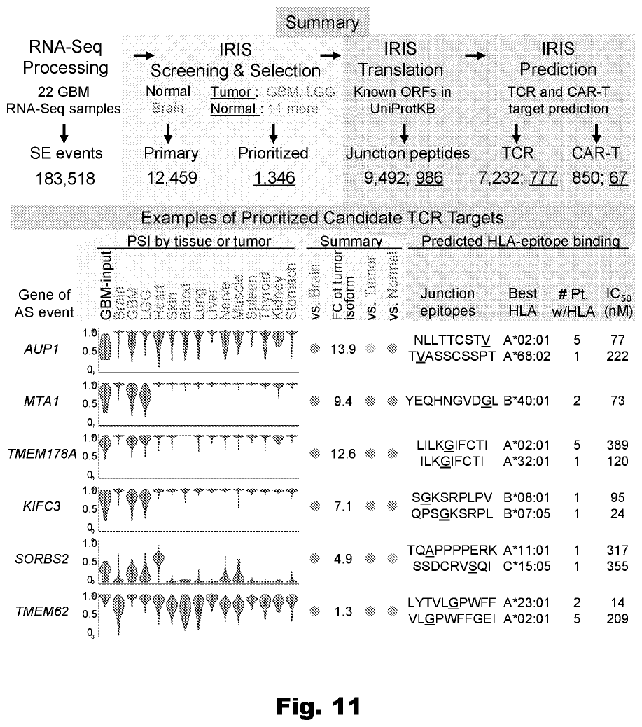

Dextramer-positive T cells are expected to contain many clonotypes, only a few of which are dominant. To discover and quantify which TCR clonotypes comprise the epitope-reactive T cells, the inventors sorted the TILs from one patient (LB2867) for cells that reacted positively with the KIGRLVTRK (SEQ ID NO:29) pMHC complex (FIG. 5B), and performed V(D)J immune profiling using single-cell RNA-Seq (scRNA-Seq) on the sorted population (FIG. 5C). Of the 325 unique TCR clonotypes, the 10 most abundant TCRs represented 86.3% of all clonotypes (Table 6), with the most frequent clonotype comprising 38.9% of all epitope-reactive T cells. This result suggests that there was clonal expansion of a select few dominant TCR clones within the tumor that were able to recognize the AS-derived epitope. To further validate the inventors' findings using complementary approaches, the inventors analyzed bulk expanded TILs using immunoSEQ and pairSEQ assays (FIG. 5C, FIG. 10). The inventors confirmed that the top 10 reported clonotypes from scRNA-Seq were present in the bulk TIL population based on the TCR β-chain CDR3 region. In addition, the pairSEQ assay, which uses statistical modeling to predict pairing of TCR α and β chains, found identically paired TCRs for seven of the top 10 TCRs from scRNA-Seq. Together, these data suggest that a select few TCR clones dominantly recognize the AS-derived epitope KIGRLVTRK (SEQ ID NO:29) in this patient.

In summary, the inventors have developed IRIS, a big data-powered platform for discovering AS-derived tumor antigens as an underexploited source of immunotherapy targets. Using IRIS followed by a dextramer-based assay, the inventors discovered and validated AS-derived tumor epitopes recognized by T cells in patients. These results provide experimental evidence for the immunogenicity of tumor antigens arising from AS and reveal novel potential targets for TCR and CAR-T therapies.

1. Methods

IRIS module for RNA-Seq data processing. IRIS accepts standard formats of raw RNA-Seq FASTQ files and/or tab-delimited files of quantified AS events (from rMATS-turbo) as input data (FIG. 3). For raw RNA-Seq data, IRIS provides a standalone pipeline that aligns RNA-Seq reads to the reference human genome hg19 using the STAR 2.5.3a (25) two-pass mode, followed by Cufflinks v2.2.1 (26) and rMATS v4.0.2 (rMATS-turbo) (19,20) for quantification of gene expression and AS events, respectively, based on the GENCODE (V26) (27) gene annotation. To quantify AS events, the inventors converted splice-junction counts in rMATS-turbo output into PSI (23) values. For each dataset, the inventors removed low-coverage AS events, defined as events with an average count of less than 10 reads for the sum of all splice junctions across all samples in that dataset (tissue/tumor type). The inventors applied this procedure to the 22 GBM samples from the UCLA cohort (BioProject: PRJNA577155), as well as to the normal and tumor samples of the reference panels used by IRIS. For the GTEx normal samples, aligned BAM files downloaded from the dbGAP repository were used directly for AS quantification.

Constructing big-data reference panels of AS events across normal human tissues and tumor samples. IRIS's big-data reference panels of normal and tumor samples are available as pre-processed, pre-indexed databases for fast retrieval by the IRIS program (FIG. 6). Specifically, 9,662 normal samples from the GTEx project (V7) (17) representing 53 tissue types of 30 histological sites were uniformly processed as described above. As shown in FIG. 6, exon-based quantification of AS events was able to distinguish samples by tissue type. Selected TCGA (16,28) tumor samples (FIG. 6C) were processed similarly to form the tumor panel. Additionally, IRIS provides a stand-alone indexing function for users to include custom normal and tumor samples in their reference panels.

IRIS module for in silico screening of tumor AS events. IRIS performs in silico screening using two-sided and one-sided t-tests to identify tumor-associated, tumor-recurrent, and tumor-specific AS events in group comparisons. To define an AS event as significantly different from a reference group (i.e., to identify tumor-associated/tumor-specific events), IRIS sets two requirements: 1) a significant p-value from the two-sided t-test (default: p<0.01), and 2) a threshold of PSI value difference (default: abs(ΔΨ)>0.05). With a slight modification, to define an AS event as recurrent in a reference group (tumor-recurrent events), IRIS compares a tumor reference group with the tissue-matched normal panel and requires: 1) a significant p-value from the one-sided t-test in the same direction as the corresponding ‘tumor-associated’ event (default: p<0.01/number of ‘tumor-associated’ events [Bonferroni correction due to large sample sizes in reference panels]), and 2) a threshold of PSI value difference (default: abs(ΔΨ)>0.05). In addition, a threshold of the number of significant comparisons against groups in the normal or tumor reference panel is used to determine whether AS-derived antigens are tumor-specific or tumor-recurrent. For each AS event, IRIS defines the ‘tumor isoform’ as the isoform that is more abundant in tumors than in the tissue-matched normal panel. Optionally, to rank or filter targets, IRIS estimates the ‘fold-change (FC) of tumor isoform’ as the FC of the tumor isoform' s proportion in tumors compared to the tissue-matched normal panel. In addition to the default ‘group mode’, IRIS can be used to screen targets for a specific patient sample through the ‘personalized mode’. This mode uses an outlier detection approach, combining a modified Tukey's rule (29) and a user-defined threshold of PSI value difference.

Identification of AS events that are prone to measurement errors due to technical variances across big-data reference panels. IRIS's big-data reference panels were constructed by integrating various large-scale datasets with distinct technical conditions, such as RNA-Seq read length (30). Such technical variances across datasets could introduce discrepancies in the quantification of AS events (30). To identify error-prone AS events, the inventors employed a data-based heuristic strategy to assess the effects of RNA-Seq read length (48 bp vs. 76 bp) and aligner (STAR vs. Tophat) on AS quantification (PSI value) (FIG. 7A). For a given tissue type (in this study, brain tissue), 10 randomly selected 76-bp RNA-Seq files from GTEx were artificially trimmed to 48 bp, and both 76- and 48-bp RNA-Seq files were aligned with STAR2.5.3a. Corresponding Tophat (v.1.4.1)-aligned 76-bp BAM files were directly downloaded from GTEx. AS events were quantified by rMATS-turbo. Events with significantly different PSI values (p<0.05, abs(ΔΨ)>0.05 from paired t-test) among RNA-Seq datasets with distinct technical conditions were included in a blacklist. Results of this analysis for GTEx normal brain samples are shown in FIG. 7B.

IRIS module for predicting AS-derived TCR and CAR-T targets. To obtain protein sequences of AS-derived tumor isoforms, IRIS generates peptides by translating splice-junction sequences into amino-acid sequences using known ORFs from the UniProtKB (31) database. Within each AS event, the splice-junction peptide sequence for the tumor isoform is compared to that of the alternative normal isoform, to ensure that the tumor isoform splice junction produces a distinct peptide.

For TCR target prediction, IRIS employs seq2HLA (32), which uses RNA-Seq data to characterize HLA class I alleles for each tumor sample. IRIS then uses IEDB API (33) predictors to obtain the putative HLA binding affinities of candidate epitopes. The IEDB ‘recommended’ mode runs several prediction tools to generate multiple predictions of binding affinity, which IRIS summarizes as a median ICso value. By default, a threshold of median(IC₅₀)<500 nM denotes a positive prediction for an AS-derived TCR target.

For CAR-T target prediction, IRIS maps AS-derived tumor isoforms to known protein extracellular domains (ECDs), as potential candidates for CAR-T therapy (FIG. 8A). Specifically, IRIS generates pre-computed annotations of protein ECDs. First, protein cellular localization information was retrieved from the UniProtKB (31) database (flat file downloaded in April 2018). ECD information was retrieved by searching for the term ‘extracellular’ in topological annotation fields, including ‘TOP_DOM’, ‘TRANSMEM’, and ‘REGION’, in the flat file. Second, BLAST (34) was used to map individual exons in the gene annotation (GENCODE V26) to proteins with topological annotations. Third, the BLAST result was parsed to create annotations of the mapping between exons and ECDs in proteins. These pre-computed annotations are queried to search for AS-derived peptides that can be mapped to protein ECDs as potential CAR-T targets.

Proteo-transcriptomics data integration for MS validation. IRIS includes an optional proteo-transcriptomics data integration function that incorporates various types of MS data, such as whole-cell proteomics, surfaceomics, or immunopeptidomics data, to validate RNA-Seq-based target discovery at the protein level (FIG. 9A). Specifically, sequences of AS-derived peptides are added to canonical and isoform sequences of the reference human proteome (downloaded from UniProtKB in September 2018). For immunopeptidomics data, fragment MS spectra are searched against the RNA-Seq-based custom proteome library with no enzyme specificity using MSGF+³⁵. The search length is limited to 7-15 amino acids. The target-decoy approach is employed to control the false discovery rate (FDR) or ‘QValue’ at 5%.

IRIS analysis of immunopeptidomics data. Published matching RNA-Seq and MS immunopeptidomics data of B-LCL-S1 and B-LCL-S2 cell lines (B lymphoblastoid cell lines from two individual donors) were retrieved from Laumont et al. (36) (GEO: GSM1641206, GSM1641207, and PRIDE: PXD001898). Raw RNA-Seq data of the JeKo-1 lymphoma cell line were obtained from the Cancer Cell Line Encyclopedia via the NCI Genomic Data Commons (available online at portal.gdc.cancer.gov/legacy-archive/). Corresponding immunopeptidomics MS data of JeKo-1 were retrieved from Khodadoust et al.³⁷ (PRIDE: PXD004746).

RNA-Seq data of the normal (B-LCL-S1, B-LCL-S2) and cancer (JeKo-1) cell lines were analyzed by IRIS as described above, with minor modifications. Specifically, AS events identified by the IRIS RNA-Seq data processing module were not subjected to the in silico screening module, but instead were directly used for the MS search. For MSGF+, FDR was set at 5%, which had the best concordance with predicted binding affinities (FIG. 9C-D). For comparison of predicted HLA binding and nonbinding peptides (FIG. 9D), a set of nonbinding peptides was created by randomly selecting peptides with median(IC₅₀)>500 nM to the same number of binding peptides (median(IC₅₀)<500 nM).

IRIS discovery of candidate TCR and CAR-T targets from 22 GBM samples. RNA-Seq samples were processed by IRIS. Detected skipped exon (SE) events were analyzed by using the IRIS screening and target prediction modules with the aforementioned default parameters. For reference panels, the ‘tissue-matched normal panel’ comprised normal brain tissue samples from GTEx; the ‘normal panel’ comprised other normal (nonbrain) tissue samples of 11 selected vital tissues (heart, skin, blood, lung, liver, nerve, muscle, spleen, thyroid, kidney and stomach) from GTEx; and the ‘tumor panel’ comprised two cohorts of brain tumor samples (GBM and LGG) from TCGA. The blacklist of AS events created for brain was applied before in silico screening by IRIS to eliminate error-prone AS events (FIG. 7).

In screening for the ‘Primary’ set of AS events, the inventors considered an event to be ‘tumor-associated’ if it was significantly different from the tissue-matched normal panel, using the default criteria described in ‘IRIS module for in silico screening of tumor AS events’. In screening for the ‘Prioritized’ set, the inventors prioritized an AS event if it was both ‘tumor-recurrent’ (significantly similar to at least 1 of 2 groups in the GBM/LGG tumor panel) and ‘tumor-specific’ (significantly different from multiple of 11 groups in the normal panel in the same direction as the tissue-matched normal panel. Here, the inventors used at least 2 groups to allow detection of AS events distinct from multiple groups in the normal panel).

When selecting potential TCR targets for dextramer validation, the inventors applied three additional criteria: 1) predicted median(IC₅₀)≤300 nM; 2) predicted binding to common HLA types, including HLA-A02:01 and HLA-A03:01; and 3) predicted binding to at least five patients in the GBM cohort. After excluding targets with low gene expression (average FPKM<5), the inventors selected seven epitopes to test for T-cell recognition by dextramer assays.

Patients. Tumor specimens were collected from 22 consenting patients with GBM who underwent surgical resection for tumor removal at the University of California, Los Angeles (UCLA; Los Angeles, Calif.). From these patients, the inventors also obtained PBMCs and TILs from two HLA-A02:01+ and four HLA-A03:01+ patients. All patients provided written informed consent, and this study was conducted in accordance with established Institutional Review Board-approved protocols.

PBMC collection. Peripheral blood was drawn from patients before surgery and diluted 1:1 in RPMI media (Thermo Fisher Scientific, cat. no. MT10041CV). PBMCs, extracted by Ficoll gradient (Thermo Fisher Scientific, cat. no. 45-001-750), were washed twice in RPMI media. Collected PBMCs were frozen in 90% human AB serum (Thermo Fisher Scientific, cat. no. MT35060CI) and 10% DMSO (Sigma, cat. no. C6295-50ML) and stored in liquid nitrogen. In parallel, PBMCs from healthy HLA-A02:01 and HLA-A03:01 donors were purchased from Bloodworks Northwest (Seattle, Wash.) or Astarte Biologics (Bothell, Wash.).

TIL collection. Surgically resected tumor samples were digested with a brain tumor dissociation kit (Miltenyi Biotec, cat. no. 130-095-42) and gentle MACS dissociator (cat. no. 130-093-235). After digestion and myelin depletion, collected cells were labeled with CD45 microbeads (cat. no. 130-045-801) and separated on Miltenyi LS columns (cat. no. 130-042-401) and MidiMACS Separator (cat no. 130-042-302). Collected CD45⁺ cells were cultured at 1×10⁶ cells/mL in X-VIVO 15 Media (Fisher Scientific, cat. no. BW04-418Q) containing 2% human AB serum with 50 ng/mL anti-CD3 antibody (BioLegend, cat. no. 317304), 1 μg/mL anti-CD28 antibody (BD Biosciences, cat. no. 555725), 1 μg/mL anti-CD49d antibody (BD Biosciences, cat. no. 555501), 300 IU/mL IL-2 (NIH, cat. no. 11697), and 10 ng/mL IL-15 (BioLegend, cat. no. 570302). Cells were expanded for 3-4 weeks and replenished with fresh media and cytokines every 2-3 days. Before freezing, expanded cells were placed in media containing 50 IU/mL IL-2 for 1-2 days and then frozen in the same freezing media as PBMCs.

Collection of tumor RNAs and RNA sequencing. RNA from freshly collected or flash-frozen tumor specimens was extracted by using the RNeasy Mini Kit (Qiagen, cat. no. 74014). Paired-end RNA-Seq was performed at the UCLA Clinical Microarray Core using an Illumina HiSeq 3000 at a read length of 2×100 bp or 2×150 bp.

Dextramer flow-cytometric analysis of PBMCs and TILs. For each AS-derived peptide selected for validation, custom-made HLA-matched MHC Class I dextramer:peptide (pMHC) complexes were purchased from Immudex (Copenhagen, Denmark). Immudex also provided pMHC complexes for common cytomegalovirus (CMV) epitopes (cat. nos. WB2132 and WC2197) and for a nonhuman epitope (NI3233) as a negative control. Each pMHC complex was purchased with two separate tags for APC or PE fluorescence labeling, to increase specificity to targeted T cells with dual labeling.

To facilitate proper gating of CD8⁺ T cells from PBMC and TIL populations, the following panel of antibodies (from BioLegend) was set up: CD3 BV605 (cat. no. 300460), CD8 FITC (cat. no. 344704), CD4 BV421 (cat. no. 317434), CD19 BV421 (cat. no. 302234), CD56 BV421 (cat. no. 362552), and CD14 BV421 (cat. no. 301828). For single-color compensation controls, OneComp eBeads were used (Thermo Fisher Scientific, cat. no. 01-1111-41).

For each set of pMHC complexes, at least 3×10⁶ cells were stained according to manufacturer's guidelines. Briefly, cells were thawed in a 37° C. water bath and washed with RPMI and D-PBS (Fisher Scientific, cat. no. MT21031CV) before staining for cell viability with the Zombie Violet Viability Kit (BioLegend, cat. no. 423113). Next, the appropriate amount of each pMHC complex in a staining buffer of D-PBS with 5% fetal bovine serum (Fisher Scientific, cat. no. MT35016CV) was added to each sample. After 10 min, the aforementioned antibody cocktail was added. After a 30-min incubation period, cells were washed twice in the same staining buffer. All samples were tested in a BD LSRII flow cytometer, and data were analyzed with FlowJo (Treestar). For gating, the lymphocyte population was first selected using forward and side scatter, and then the BV421-negative population was gated out (i.e. excluding dead cells and the CD14, CD19, CD56, and CD4 populations) before selecting the CD3⁺CD8⁺population. To set for proper gating of dextramer-positive cells, the inventors used cells that were stained with the full antibody panel but no pMHC complexes, and cells that were given the nonhuman pMHC complex.

TCR sequencing using scRNA-Seq. Cells were stained by following the dextramer procedure with PE-conjugated pMHC complexes only. Cells were sorted by using the BD FACSAria flow cytometer, and PE⁺ cells were collected. V(D)J immune profiling of sorted cells was done with scRNA-Seq, using the 10× Genomics Chromium Single Cell Immune Profiling Workflow at the UCLA Clinical Microarray Core. Each T cell was encapsulated in an oil emulsion droplet with a barcoded gel bead, and reverse transcription was performed to create a barcoded cDNA library. The V(D)J-enriched and gene expression libraries were sequenced using the 10X Genomics Chromium Controller. After sequencing, the Cell Ranger pipeline was used to align reads, filter, count barcodes and assign unique molecular identifiers.

Next-generation immune repertoire sequencing using the immunoSEQ platform. To assess the T-lymphocyte repertoire of bulk expanded TIL populations, the inventors used the immunoSEQ assay (Adaptive Biotechnologies). This multiplex PCR system uses a mixture of primers that target the rearranged V and J segments of the CDR3 region to assess TCR diversity within a given sample. Genomic DNA from each sample was extracted by using the QlAamp DNA Blood Midi Kit (Qiagen, cat. no. 51185). The inventors provided at least 1 μg of DNA (˜60,000 cells) from each sample to Adaptive Biotechnologies for sequencing at a deep resolution. Resulting sequencing data were analyzed with the immunoSEQ Analyzer Platform (Adaptive Biotechnologies).

High-throughput αβ TCR pairing using the pairSEQ platform. The inventors provided Adaptive Biotechnologies with frozen bulk expanded TIL samples for their pairSEQ assay, to predict which α and β chains may pair to form a functional TCR. Briefly, T cells were randomly distributed into wells of a 96-well plate. The mRNA was extracted, converted to cDNA, and amplified by using TCR-specific primers. The cDNA of T cells from each well was given a specific barcode, and all wells were pooled together for sequencing. Each TCR sequence was mapped back to the original well through computational demultiplexing. Putative TCR pairs were identified by examining whether a sequenced TCR α chain was frequently seen to share the same well with a specific sequenced TCR β chain, above statistical noise.

The 22 UCLA GBM RNA-Seq data generated for this study were uploaded to BioProject database (BioProject: PRJNA577155). For the IRIS proteo-transcriptomics analysis, matching RNA-Seq data and MS immunopeptidomics data of B-LCL-S 1 and B-LCL-S2 cell lines were retrieved from Laumont et al. (GEO: GSM1641206, GSM1641207 and PRIDE: PXDO01898). Raw RNA-Seq data of the JeKo-1 lymphoma cell line were obtained from the Cancer Cell Line Encyclopedia via the NCI Genomic Data Commons. Corresponding MS immunopeptidomics MS data of JeKo-1 were retrieved from Khodadoust et al. (PRIDE: PXD004746).

Lengthy table referenced here
US20220380937A1-20221201-T00001
Please refer to the end of the specification for access instructions.

Lengthy table referenced here
US20220380937A1-20221201-T00002
Please refer to the end of the specification for access instructions.

Lengthy table referenced here
US20220380937A1-20221201-T00003
Please refer to the end of the specification for access instructions.

Lengthy table referenced here
US20220380937A1-20221201-T00004
Please refer to the end of the specification for access instructions.

Lengthy table referenced here
US20220380937A1-20221201-T00005
Please refer to the end of the specification for access instructions.

Lengthy table referenced here
US20220380937A1-20221201-T00006
Please refer to the end of the specification for access instructions.

Lengthy table referenced here
US20220380937A1-20221201-T00007
Please refer to the end of the specification for access instructions.

Lengthy table referenced here
US20220380937A1-20221201-T00008
Please refer to the end of the specification for access instructions.

Lengthy table referenced here
US20220380937A1-20221201-T00009
Please refer to the end of the specification for access instructions.

Lengthy table referenced here
US20220380937A1-20221201-T00010
Please refer to the end of the specification for access instructions.

Lengthy table referenced here
US20220380937A1-20221201-T00011
Please refer to the end of the specification for access instructions.

Lengthy table referenced here
US20220380937A1-20221201-T00012
Please refer to the end of the specification for access instructions.

Lengthy table referenced here
US20220380937A1-20221201-T00013
Please refer to the end of the specification for access instructions.

Lengthy table referenced here
US20220380937A1-20221201-T00014
Please refer to the end of the specification for access instructions.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Sun, C., Mezzadra, R. & Schumacher, T. N. Immunity 48, 434-452 (2018).
2. Rosenberg, S. A. & Restifo, N. P. Science 348, 62-68 (2015).
3. Yarchoan, M., Johnson, B. A., Lutz, E. R., Laheru, D. A. & Jaffee, E. M. Nat. Rev. Cancer 17, 209-222 (2017).
4. Schumacher, T. N. & Schreiber, R. D. Science 348, 69-74 (2015).
5. Lee, C.-H., Yelensky, R., Jooss, K. & Chan, T. A. Trends Immunol. 39, 536-548 (2018).
6. Coulie, P. G., Van den Eynde, B. J., van der Bruggen, P. & Boon, T. Nat. Rev. Cancer 14, 135-146 (2014).
7. Nat. Biotechnol. 35, 97-97 (2017).
8. Vitiello, A. & Zanetti, M. Nat. Biotechnol. 35, 815-817 (2017).
9. Marty, R. et al. Cell 171, 1272-1283.e15 (2017).
10. Ott, P. A. et al. Nature 547, 217-221 (2017).
11. Nemecek, R. et al. Nature 547, 222-226 (2017).
12. Carreno, B. M. et al. Science 348, 803-8 (2015).
13. Thibault, P. et al. Sci. Transl. Med. 10, eaau5516 (2018).
14. Smart, A. C. et al. Nat. Biotechnol. 36, 1056 (2018).
15. Zhang, M. et al. Nat. Commun. 9, 3919 (2018).
16. Kahles, A. et al. Cancer Cell 34, 211-224.e6 (2018).
17. GTEx Consortium, T.Gte. Nat. Genet. 45, 580-5 (2013).
18. Cancer Genome Atlas Research Network, J. N. et al. Nat. Genet. 45, 1113-20 (2013).
19. Shen, S. et al. Proc. Natl. Acad. Sci. 111, E5593-E5601 (2014).
20. Xie, Z. & Xing, Y. (2018).at <world wide web at rnaseq-mats.sourceforge.net/rmats4.0.2/>
21. Bonifant, C. L., Jackson, H. J., Brentjens, R. J. & Curran, K. J. Mol. Ther. Oncolyt. 3, 16011 (2016).
22. Abelin, J. G. et al. Immunity 46, 315-326 (2017).
23. Katz, Y., Wang, E. T., Airoldi, E. M. & Burge, C. B. Nat. Methods 7, 1009-15 (2010).
24. Hadrup, S. R. & Schumacher, T. N. Cancer Immunol. Immunother. 59, 1425-1433 (2010).
25. Dobin, A. et al. Bioinformatics 29, 15-21 (2013).
26. Trapnell, C. et al. Nat. Protoc. 7, 562-78 (2012).
27. Harrow, J. et al. Genome Res. 22, 1760-1774 (2012).
28. McLendon, R. et al. Nature 455, 1061-1068 (2008).
29. Tukey, J. W. (John W. (Addison-Wesley Pub. Co: 1977).
30. Baruzzo, G. et al. Nat. Methods 14, 135-139 (2017).
31. UniProt Consortium, T. Nucleic Acids Res. 46, 2699-2699 (2018).
32. Boegel, S. et al. Genome Med. 4, 102 (2012).
33. Vita, R. et al. Nucleic Acids Res. 43, D405—D412 (2015).
34. Altschul, S.F., Gish, W., Miller, W., Myers, E.W. & Lipman, D.J. J. Mol. Biol. 215, 403-410 (1990).
35. Kim, S. & Pevzner, P.A. Nat. Commun. 5, 5277 (2014).
36. Laumont, C.M. et al. Nat. Commun. 7, 10238 (2016).
37. Khodadoust, M. S. et al. Nature (2017).doi:10.1038/nature21433

LENGTHY TABLES
The patent application contains a lengthy table section. A copy of the table is available in electronic form from the USPTO web site (https://seqdata.uspto.gov/?pageRequest=docDetail&DocID=US20220380937A1). An electronic copy of the table will also be available from the USPTO upon request and payment of the fee set forth in 37 CFR 1.19(b)(3).

What is claimed is:

1. A method to synthesize an antigenic peptide, comprising:
identifying alternative splice events in RNA-seq data derived from neoplastic tissue;
obtaining a reference panel of alternative splicing events that includes splice junction data, wherein the reference panel of alternative splicing events is derived from healthy matched tissue, other tissues of the body, or a second neoplastic tissue that is similar;
detecting a neoplastic alternative splicing event in the neoplastic tissue by comparing the alternative splice events derived from neoplastic tissue with the reference panel of alternative splicing events;
selecting an alternative isoform from the neoplastic tissue RNA-seq data that is detected to have the neoplastic alternative splicing event;
generating a peptide derived based on a nucleotide sequence that spans across a splice junction of the detected neoplastic alternative splicing event of the selected alternative isoform.

2. The method as in claim 1, wherein the selected isoform is selected based on the neoplastic splicing event being present at a greater level in the neoplastic tissue as compared to the healthy matched tissue or the other tissues of the body of the reference panel.

3. The method as in claim 1 or 2, wherein the selected isoform is selected based on the neoplastic splicing event being present at a greater level in the second neoplastic tissue as compared to the healthy matched tissue or the other tissues of the body of the reference panel.

4. The method as in claim 1, 2 or 3, wherein the alternative splice event is a skipped exon, an included exon, an alternative 3′ splice site, and alternative 5′ splice site, or a retained intron.

5. The method as in any of claims 1 to 4, wherein the alternative splice events in the RNA-seq data are identified using the rMATS package.

6. The method as in any of claims 1 to 5, wherein the neoplastic alternative splicing event is determined by the relative abundance or prevalence of alternative isoforms in the neoplastic tissue as compared to the reference tissue panel.

7. The method as in any of claims 1 to 6, wherein the reference tissue panel includes alternative splicing events from healthy tissue having the same tissue origin as the neoplastic tissue.

8. The method as in claim 6 or 7, wherein the relative abundance of alternative isoform is determined by the relative expression of the alternative isoform in the neoplastic tissue, as compared to the relative expression of the alternative isoform in the reference tissue panel.

9. The method as in claim 6 or 7, wherein the prevalence of the alternative isoform is determined by the number of samples expressing the alternative isoform within a neoplastic tissue panel, as compared to the number of samples expressing the alternative isoform within the reference tissue panel.

10. The method as in any of claims 1 to 9, wherein the selection of at least one alternative isoform is based upon a statistical inference of the significance of the neoplastic alternative splicing event.

11. The method as in any of claims 1 to 10, wherein the generated peptide is determined to be a T Cell Receptor (TCR) target.

12. The method as in claim 11, wherein the generated peptide has computed median HLA binding affinity (IC₅₀) less than 500 nM.

13. The method as in any of claims 1 to 2, wherein the generated peptide is a part of an extracellular domain.

14. The method as in any of claims 1 to 13, wherein the generated peptide was identified in mass spectrometry data.

15. The method as in any of claims 1 to 14, wherein the generated peptide is synthesized via solid-phase peptide synthesis.

16. The method as in any of claims 1 to 14, wherein the generated peptide is synthesized via molecular expression in a host cell.

17. The method as in any of claims 1 to 16, wherein the generated peptide is utilized in an assay to determine peptide immunogenicity.

18. The method as in any of claims 1 to 16, wherein the generated peptide is utilized in an assay to determine recognition by T cells.

19. The method as in any of claims 1 to 16, wherein the generated peptide is utilized in a peptide vaccine for treatment of the neoplasm.

20. The method as in any of claims 1 to 16, wherein the generated peptide is utilized to develop modified T cell receptors of T cells.

21. The method as in any of claims 1 to 16, wherein the generated peptide is utilized to develop antibodies.

22. The method as in any of claims 1 to 16, wherein the generated peptide is utilized develop chimeric antigen receptors of T cells.

23. The method as in any of claims 1 to 23, wherein the neoplasm is one of: acute lymphoblastic leukemia (ALL), acute myeloid leukemia (AML), anal cancer, astrocytomas, basal cell carcinoma, bile duct cancer, bladder cancer, breast cancer, Burkitt's lymphoma, cervical cancer, chronic lymphocytic leukemia (CLL) chronic myelogenous leukemia (CIVIL), chronic myeloproliferative neoplasms, colorectal cancer, diffuse large B-cell lymphoma, endometrial cancer, ependymoma, esophageal cancer, esthesioneuroblastoma, Ewing sarcoma, fallopian tube cancer, follicular lymphoma, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T cell lymphoma, testicular cancer, thymoma, thyroid cancer, uterine cancer, vaginal cancer, or vascular tumors.

24. The method as in any of claims 1 to 23, wherein the neoplastic tissue is sourced from a tumor biopsy, a nodal biopsy, a surgical resection, or a liquid/soft biopsy.

25. An engineered T-cell Receptor (TCR) comprising:
a TCR alpha (TCR-a) CDR3 comprising an amino acid sequence with at least 90% sequence identity to SEQ ID NO:30 and a TCR beta (TCR-b) CDR3 comprising an amino acid sequence with at least 90% sequence identity to SEQ ID NO:31;
a TCR alpha (TCR-a) CDR3 comprising an amino acid sequence with at least 90% sequence identity to SEQ ID NO:32 and a TCR beta (TCR-b) CDR3 comprising an amino acid sequence with at least 90% sequence identity to SEQ ID NO:33;
a TCR alpha (TCR-a) CDR3 comprising an amino acid sequence with at least 90% sequence identity to SEQ ID NO:34 and a TCR beta (TCR-b) CDR3 comprising an amino acid sequence with at least 90% sequence identity to SEQ ID NO:35;
a TCR alpha (TCR-a) CDR3 comprising an amino acid sequence with at least 90% sequence identity to SEQ ID NO:36 and a TCR beta (TCR-b) CDR3 comprising an amino acid sequence with at least 90% sequence identity to SEQ ID NO:37;
a TCR alpha (TCR-a) CDR3 comprising an amino acid sequence with at least 90% sequence identity to SEQ ID NO:38 and a TCR beta (TCR-b) CDR3 comprising an amino acid sequence with at least 90% sequence identity to SEQ ID NO:39;
a TCR alpha (TCR-a) CDR3 comprising an amino acid sequence with at least 90% sequence identity to SEQ ID NO:40 and a TCR beta (TCR-b) CDR3 comprising an amino acid sequence with at least 90% sequence identity to SEQ ID NO:41;
a TCR alpha (TCR-a) CDR3 comprising an amino acid sequence with at least 90% sequence identity to SEQ ID NO:42 and a TCR beta (TCR-b) CDR3 comprising an amino acid sequence with at least 90% sequence identity to SEQ ID NO:43; or
a TCR-a and TCR-b CDR3 comprising an amino acid sequence with at least 90% sequence identity to a TCR-a and TCR-b CDR3 pair from a clonotype listed in Table 6.

26. The TCR of claim 25, wherein the TCR comprises:
a TCR alpha (TCR-a) variable region comprising an amino acid sequence with at least 80% sequence identity to SEQ ID NO:44 and a TCR beta (TCR-b) variable region comprising an amino acid sequence with at least 80% sequence identity to SEQ ID NO:45;
a TCR alpha (TCR-a) variable region comprising an amino acid sequence with at least 80% sequence identity to SEQ ID NO:46 and a TCR beta (TCR-b) variable region comprising an amino acid sequence with at least 80% sequence identity to SEQ ID NO:47; or
a TCR alpha (TCR-a) variable region comprising an amino acid sequence with at least 80% sequence identity to SEQ ID NO:48 and a TCR beta (TCR-b) variable region comprising an amino acid sequence with at least 80% sequence identity to SEQ ID NO:49.

27. The TCR of claim 25 or 26, wherein the TCR comprises or consists of a bispecific TCR.

28. The TCR of claim 27, wherein the bispecific TCR comprises an scFv that targets or selectively binds CD3.

29. The TCR of any one of claims 25-28, wherein the soluble TCR is further defined as a single-chain TCR (scTCR), wherein the a chain and the β chain are covalently attached via a flexible linker.

30. The TCR of 2526any one of claims 25-29, wherein the TCR comprises a modification or is chimeric.

31. One or more nucleic acids encoding the TCR of claim 25 or 30.

32. The nucleic acid(s) of claim 31, wherein the nucleic acid comprises a cDNA encoding the TCR.

33. A nucleic acid vector comprising the nucleic acid(s) of claim 31 or 32.

34. The vector of claim 33, wherein the vector comprises the TCR alpha and TCR beta genes.

35. A cell comprising the TCR of claim 25 or 30, the nucleic acid(s) of claim 31 or 32, or the vector of claim 33 or 34.

36. The cell of claim 35, wherein the cell is an immune cell.

37. The cell of claim 35 or 36, wherein the cell comprises a stem cell, progenitor cell, T cell, NK cell, invariant NK cell, NKT cell, mesenchymal stem cell (MSC), induced pluripotent stem (iPS) cell, regulatory T cell, CD8+ T cell, CD4+ T cell, or γδ T cell.

38. The cell of claim 37, wherein the cell comprises a hematopoietic stem or progenitor cell, a T cell, or an induced pluripotent stem cell (iPSC).

39. The cell of any one of claims 35-38, wherein the cell is autologous.

40. The host of any one of claims 35-38, wherein the cell is allogeneic.

41. The cell of any one of claims 35-40, wherein the cell is isolated from a cancer patient.

42. The cell of any one of claims 35-41, wherein the cell is a HLA-A type.

43. The cell of claim 42, wherein the cell is a HLA-A*03:01 type, HLA-A*01:01, or HLA-A*02:01.

44. A composition comprising the cell of any one of claims 35-43.

45. The composition of claim 44, wherein the composition has been determined to be serum-free, mycoplasma-free, endotoxin-free, and/or sterile.

46. A method comprising transferring the nucleic acid of any one of claim 32 or 33 or the vector of claim 34 into a cell.

47. The method of claim 46, wherein the method further comprises culturing the cell in media, incubating the cell at conditions that allow for the division of the cell, screening the cell, and/or freezing the cell.

48. A method for treating brain cancer in a subject comprising administering the composition of claim 44 or 45 to a subject.

49. The method of claim 48, wherein the brain cancer comprises glioblastoma or glioma.

50. The method of any one of claims 48-49, wherein the subject has previously been treated for the cancer.

51. The method of claim 50, wherein the subject has been determined to be resistant to the previous treatment.

52. The method of any one of claims 48-51, wherein the method further comprises the administration of an additional therapy.

53. The method of any one of claims 48-52, wherein the cancer comprises stage I, II, III, or IV cancer.

54. The method of any one of claims 48-53, wherein the cancer comprises metastatic and/or recurrent cancer.

55. A peptide from the TRIM11 protein comprising at least 6 contiguous amino acids from the TRIM11 and comprising the amino acids QD, which correspond to the amino acids at positions 168-169 of SEQ ID NO:1.

56. A peptide from the RCOR3 protein comprising at least 6 contiguous amino acids from the RCOR3 and comprising the amino acids QG, which correspond to the amino acids at positions 358-359 of SEQ ID NO:2.

57. A peptide from the FAM76B protein comprising at least 6 contiguous amino acids from the FAM76B and comprising the amino acids DS, which correspond to the amino acids at positions 230-231 of SEQ ID NO:3.

58. A peptide from the SLMAP protein comprising at least 6 contiguous amino acids from the SLMAP and comprising the amino acids NP, which correspond to the amino acids at positions 332-333 of SEQ ID NO:4.

59. A peptide from the TMEM62 protein comprising at least 6 contiguous amino acids from the TMEM62 and comprising the amino acids LG, which correspond to the amino acids at positions 495-496 of SEQ ID NO:5.

60. A peptide from the PLA2G6 protein comprising at least 6 contiguous amino acids from the PLA2G6 and comprising the amino acids RL, which correspond to the amino acids at positions 395-396 of SEQ ID NO:6.

61. A peptide comprising at least 6 contiguous amino acids from one of SEQ ID NOS:786 or 1364-1395.

62. A peptide having at least 70% sequence identity to a peptide of SEQ ID NO:786 or 1364-1395.

63. A peptide comprising at least 6 contiguous amino acids from a peptide of Table 1a, Table 1b, Table 1c, or 4, wherein the peptide comprises an alternative splice site junction.

64. A peptide comprising at least 6 contiguous amino acids encoded by an alternatively spliced nucleic acid, wherein the at least 6 contiguous amino acids are encoded on a nucleic acid that comprises an alternative splice site junction, and wherein the alternative splice site junction is an AS event selected from an AS event in Table 3a or 3b.

65. The peptide of claim 64, wherein the AS event is selected from an AS event in Table 3 a.

66. The peptide of claim 65, wherein the AS event is selected from an AS event in Table 3b.

67. The peptide of claim 55, wherein the peptide comprises an amino acid sequence selected from SEQ ID NO:7-9.

68. The peptide of claim 56, wherein the peptide comprises an amino acid sequence of SEQ ID NO:10.

69. The peptide of claim 57, wherein the peptide comprises an amino acid sequence of SEQ ID NO:11 or 12.

70. The peptide of claim 58, wherein the peptide comprises an amino acid sequence selected from SEQ ID NO:13-15.

71. The peptide of claim 59, wherein the peptide comprises an amino acid sequence selected from SEQ ID NO:16-22.

72. The peptide of claim 60, wherein the peptide comprises an amino acid sequence selected from SEQ ID NO:23-29.

73. The peptide of any one of claims 55-72, wherein the peptide comprises at least 10 amino acids.

74. The peptide of any one of claims 55-72, wherein the peptide consists of 10 amino acids.

75. The peptide of any one of claims 55-74, wherein the peptide is less than 20 amino acids in length.

76. The peptide of any one of claims 55-75, wherein the peptide is modified.

77. The peptide of claim 76, wherein the modification comprises conjugation to a molecule.

78. The peptide of claim 76 or 77, wherein the molecule comprises an antibody, a lipid, an adjuvant, or a detection moiety.

79. A composition comprising the peptide of any one of claims 55-78.

80. The composition of claim 79, wherein the composition is formulated as a vaccine.

81. The composition of claim 79 or 80, wherein the composition further comprises an adjuvant.

82. A nucleic acid encoding for the peptide of any one of claims 55-78.

83. An expression vector comprising the nucleic acid of claim 82.

84. A host cell comprising the nucleic acid of claim 82 or the expression vector of claim 83.

85. An in vitro isolated dendritic cell comprising the peptide of any one of claims 55-78, the nucleic acid of claim 82, or the expression vector of claim 83.

86. The dendritic cell of claim 85, wherein the dendritic cell comprises a mature dendritic cell.

87. The dendritic cell of claim 85 or 86, wherein the cell is a cell with an HLA type selected from HLA-A, HLA-B, or HLA-C.

88. The dendritic cell of claim 85 or 86, wherein the cell is a cell with an HLA type selected from HLA-A*02:01, HLA-A*03:01, HLA-A*23:01, HLA-A*68:02, HLA-B*07:05, HLA-B*18:01, HLA-B*40:01, HLA-C*03:03, HLA-C*14:02, or HLA-C*15:02.

89. A method of making a cell comprising transferring the nucleic acid of claim 82 or the expression vector of claim 83 into the cell.

90. The method of claim 89, wherein the method further comprises isolating the expressed peptide or polypeptide.

91. An in vitro method for making a dendritic cell vaccine comprising contacting a mature dendritic cell in vitro with a peptide of any one of claims 55-78.

92. The method of claim 91, wherein the method further comprises screening the dendritic cell for one or more cellular properties.

93. The method of claim 91 or 92, wherein the method further comprises contacting the cell with one or more cytokines or growth factors.

94. The method of claim 93, wherein the one or more cytokines or growth factors comprises GM-CSF.

95. The method of claim 92, wherein the cellular property comprises cell surface expression of one or more of CD86, HLA, and CD14.

96. The method of any one of claims 91-95, wherein the dendritic cell is derived from a CD34+hematopoietic stem or progenitor cell.

97. The method of any one of claims 91-95, wherein the dendritic cell is derived from a peripheral blood monocyte (PBMC).

98. The method of any one of claims 91-95, wherein the dendritic cells are cells in which the DCs are derived are isolated by leukaphereses.

99. An in vitro composition comprising a dendritic cell and the peptide of any one of claims 55-78.

100. The composition of claim 99, wherein the composition further comprises one or more cytokines, growth factors, or adjuvants.

101. The composition of claim 100, wherein the composition comprises GM-CSF.

102. The composition of claim 101, wherein the peptide and GM-CSF are linked.

103. The composition of claim any one of claims 99-103, wherein the composition is determined to be serum-free, mycoplasma-free, endotoxin-free, and sterile.

104. The composition of any one of claims 99-103, wherein the peptide is on the surface of the dendritic cell.

105. The composition of claim 104, wherein the peptide is bound to a MEW molecule on the surface of the dendritic cell.

106. The composition of any one of claims 99-105, wherein the composition is enriched for dendritic cells expressing CD86 on the surface of the cell.

107. The composition of any one of claims 99-106, wherein the dendritic cell comprises a monocyte-derived dendritic cell.

108. The composition of any one of claims 99-106, wherein the dendritic cell is derived from a CD34+ hematopoietic stem or progenitor cell.

109. The composition of any one of claims 99-106, wherein the dendritic cell is derived from a peripheral blood monocyte (PBMC).

110. The composition of any one of claims 99-109, wherein the dendritic cells or the cells in which the DCs are derived from are isolated by leukaphereses.

111. An engineered T-cell Receptor (TCR) or chimeric antigen receptor (CAR) that specifically recognizes the peptide of any one of claims 55-78.

112. A cell comprising the TCR or CAR of claim 111.

113. The cell of claim 112, wherein the cell comprises at least one TCR and at least one CAR and wherein the TCR and CAR each recognize a different peptide.

114. The cell of claim 112 or 113, wherein the cell comprises a stem cell, a progenitor cell, or a T cell.

115. The cell of claim 114, wherein the cell comprises a hematopoietic stem or progenitor cell, a T cell, or an induced pluripotent stem cell (iPSC).

116. An antibody or antigen binding fragment thereof that specifically recognizes the peptide of any one of claims 55-78.

117. A method of treating a subject for brain cancer comprising administering the peptide of any one of claims 55-78, the composition of any one of claim 79-81 or 99-110, the dendritic cell of any one of claims 85-88, or the cell of any one of claims 112-115 or the antibody or antigen binding fragment of claim 116.

118. The method of claim 117, wherein the method comprises administering a cell or a composition comprising a cell and wherein the cell comprises an autologous cell.

119. The method of claim 117 or 118, wherein the cancer comprises glioblastoma or glioma.

120. The method of any one of claims 117-119, wherein the subject has previously been treated for the cancer.

121. The method of claim 120, wherein the subject has been determined to be resistant to the previous treatment.

122. The method of any one of claims 117-121, wherein the method further comprises the administration of an additional therapy.

123. The method of any one of claims 117-122, wherein the cancer comprises stage I, II, III, or IV cancer.

124. The method of any one of claims 117-123, wherein the cancer comprises metastatic and/or recurrent cancer.

125. A method of activating or expanding peptide-specific T cells comprising contacting a starting population of T cells from a mammalian subject and preferably from a blood sample from the mammalian subject cells ex vivo with the peptide of any one of claims 55-78 thereby activating, stimulating proliferation, and/or expanding peptide-specific T cells in the starting population.

126. The method of claim 125, wherein contacting is further defined as co-culturing the starting population of T cells with antigen presenting cells (APCs), wherein the APCs can present the peptide of any one of claims 55-78 on their surface.

127. The method of claim 126, wherein the APCs are dendritic cells.

128. The method of claim 127, wherein the dendritic cells are autologous dendritic cells obtained from the mammalian subject.

129. The method of claim 125, wherein contacting is further defined as co-culturing the starting population of T cells with artificial antigen presenting cells (aAPCs).

130. The method of claim 129, wherein the artificial antigen presenting cells (aAPCs) comprise or consist of poly(lactide-co-glycolide) (PLGA), K562 cells, paramagnetic beads coated with CD3 and CD28 agonist antibodies, beads or microparticles coupled with an HLA-dimer and anti-CD28, or nanosize-aAPCs (nano-aAPC) that are preferably less than 100 nm in diameter.

131. The method of any one of claims 125-130, wherein the T cells are CD8+ T cells or CD4+ T cells.

132. The method of any one of claims 125-131, wherein the T cells are cytotoxic T lymphocytes (CTLs).

133. The method of any one of claims 125-132, wherein the starting population of cells comprises or consists of peripheral blood mononuclear cells (PBMCs).

134. The method of claim 133, wherein the method further comprises isolating or purifying the T cells from the peripheral blood mononuclear cells (PBMCs).

135. The method of any one of claims 125-134, wherein the mammalian subject is a human.

136. The method of any one of claims 125-135, wherein the method further comprises reinfusing or administering the activated or expanded peptide-specific T cells to the subject.

137. A peptide-specific T cell activated or expanded according to any one of claims 125-136.

138. A pharmaceutical composition comprising the peptide-specific T cells activated or expanded according to any one of claims 125-136.
